SEQUENCE LISTING (132 sequences)

SEQ ID NO: 1 - protein, 230 aa, Erysipelatoclostridium ramosum
MDVYEIKIKLYLLKDIKIEETQTYLAYFIDSVMVKDNMFLGIHETNQYKFYTFDSLYPLAKNGVYQKDNSYMFRIRTLDYQLAQYLYDTLAKNRTKEFQGLTAEVKIIKPKLIKKIYTLTPVILKTEQGYWKNSIKTEDFEKRLKTNLIKKYKDITGEEINEDFQLYYQINFKNKVPVSRKYKGIKLLGDMIELEIAENDNAQKLAFLAIGSGLLEMNARGFGFVNYIYY

SEQ ID NO: 2 - protein, 582 aa, Erysipelatoclostridium ramosum
MKGGYIMLDECLRIFKQELKDNTNLVLNTIILADGDYVLVHDDGTYDVEKIKYSKKDHCFIEKPEDDLYEKLCFYDYQSQVIDTQKTFDLPHKLILSNNYLSYFVKKEKLNNKKKINDATEKYFLALSNPRAKYNGKDLEMYNFVTQNISEVDKRRLLQNKQWLKQYIYNLDGVDYNEKDYLKIFFEADYHQYINEGNRYLLTKIFNCNDYNTYENETVYGVPNYNMQLNSNKPFLKNMTRMHKVPFKLSLEEALIQKQFFDYLLNYANKGKTNIYISSNENDTKRIYFLDNTKKIKKGFNGLYLKIKKDKELKIEYIDTVTDYKEYLSPMFNFRNVIGAFDDEYYQEYNKRNEVEKLLNDVLFSKFLINNYFTDPDSIISIKGENIDVYKKNLITCREAIFAWIHVGAVGNIKNILPKAALETTIHSIKMGHIKLAQRQFNLYFAINEYFNKQENGMKDLRESIRTKINSQQQMKIENDLEYSFAVGQAIAYLQSKSKAKNKTQDFINQFITIKRDDSMKSKLKNLYLHYNYALSTGKTRFNMLFGMIMEYQSDQKINQNMLIAGYIGENLIYEKGENENG

SEQ ID NO: 3 - protein, 305 aa, Erysipelatoclostridium ramosum
MDKRVYGVIGVSSKMANWNADFSGYPRSLSDGTIFGSDKALKYPMRKMWDNEGKKVLYMKSMNFSKKNDTITLVPNSLKERYEKLFAVDDLKKCKDNKEVLTNLMTAVDVKNFGATFAEAKNNISITGAVQFGQGINKYGDSDLETMQILSPFRDTKDAEAKNSTIGTKKIINEAHYFYPFSINPSAYKELVALGVTDGYTEEDYLNFKRTALVAATSFSSNAKEGCQNEFALFVETKLDTYLPNLSEYISFEKADINKIKIECNMLNELEDILNIEIYYNPETTVLESNLEKAKIYNLITKKEV

SEQ ID NO: 4 - protein, 232 aa, Erysipelatoclostridium ramosum
MQALKFTLSGKTAFFKRPEVNTYYFTFGNIHRVALLGIFGAILGYQGYNNTIELPEFYQKLKDIKLSILPQNEKGYFSKKIQYYNNSVGYASKQQGGNLIIKEQWLENVCWDIVVLLDNEEANKLAKAIINKKCIFIPYLGKNDHPANITDPVIVDLQKRDIDKETLDCLFPSGNFKKAEDVYVNFYKYQESLPIGLNEKTGLYEYMTFIHTNFYVDKGAVYSFNDKNIVFY

SEQ ID NO: 5 - protein, 835 aa, Erysipelatoclostridium ramosum
MDIDKYLVSLEKIIINIDQCYGHRDDDHQETINEHIQLCTKYLKEIFKLKKLDSILKSFNISLGEGLSNEGKEMFNELFFNTITFHDTGKINPFFQKNKMNNPVMNYLNPPKNLKSDHSKLSAYIYLGTYLNKLNRLSRNDIGLIKPIVYINAFVISRHHAKIDDFRTYFMDKLDSDDILEKRIIEWMNDEKFISQFTEKLSLDSLNTDEYNYIFEKLEKYEMNIQIDLYAYVRFLYSLLVFCDYYATSEFYGDNKNNVMLKDADFGEIIELYDNSELVTKIRKNDIGKEKINALRTKVFLEAERNLLENIDKNIFYLEAPTGSGKSNTALNLSLRLIENDDSLNKIIYVYPFNTLVEQNLASLNKIFGNSKAMNNIAVVNSTTPLKKDEDENFSDEYQKALLDRQFLTYPIILTTHVGLFDTFFGNNRESAFGLCQYANSVIVLDEIQNYRIEIWNEMIIFLKEFAQILNLKIIIMSATLPDLELLTDDRSNSVSLITNPQEYFLNPIFKDRVKTDYSLINVENTEEALLNHVLEMNKLKKKIMIEFIVRKSAEKFYRQLKEIELDCEVLFISGFDSVLEREKIISTVKTSKHLILVATQVIEAGVDIDMDIGYKDISKLDSEEQFMGRINRSCKEDGKGIVYFFNLNKATNIYKGDERVAQKNLTLFNDDMKKVLLKKNFNYYYQKLLKTLANRAKKPEEKNVPNFFRESVACLDYQAVSKRMELILDTRDRVTIFLGRTVINIDGEEFDGKDIWNQYVDLLKNNEMDYAKKVYELSVVKSMMNYFMYQVIKKNQFDFKEQVGDIYYIENGDDYILDGKLNTALFETEDELFI

SEQ ID NO: 6 - protein, 164 aa, Erysipelatoclostridium ramosum
MEVNGTLINYYFHCQRQCYLHGNRLNLEDESENVKIGKALHEEKYKDDKNSEIAIDNIRLDKLTAKYLTETKKSDADLVAAKWQLLYYLKILKSKGIIRKGRIEVIEKNKQNKSFIEVELTEIEEKELDKIVIKIKELLENDQIPPVLNENKCKKCAYYAYCYI

SEQ ID NO: 7 - protein, 332 aa, Erysipelatoclostridium ramosum
MKSTKYITSMGDLKRKDDSLCFRKSGKNIYLPVENIKEIYCLNEISLNTKLLDFISSKNIIIHFFNYYQGYSGTFYPKDKYTSGKLLIKQVETFNNYRIDIAKAIVNGIAINIDETLYHYYKHGKNEVKETIDWLRKEVPKRLKKAEDIKMIMAVEGEIWQRFYSMFIYILPEDFIMNKRVKRPPDNPINALISFGNTLLYTKTISAIYQTHLNQSISFLHEPTEQRFSLSLDLSEVFKPIIVFKTIFELVNTKRLTVEKHFDKKTNYCLLNDKGRDIFIEAFENRMETKFLHTKLKRKITYKTAIKYDGYKLIKTIFENKLFIPFSIKDKY

SEQ ID NO: 8 - protein, 95 aa, Erysipelatoclostridium ramosum
MKIKNHNYVIVCYDIGEKRVNKIFKICKKYLPHYQYSIFKGPITPSKLILLKKELKKAINKEEDCVSIIKLQSEDSFDEEILGSQKEGNEDSLII

SEQ ID NO: 9 - DNA, 693 bp, Erysipelatoclostridium ramosum
  source 1..693: mol_type = genomic DNA
atggatgtatatgaaataaagataaaattatatttattgaaagatataaaaattgaagaaacacagacttatttagcatactttattgatagtgtaatggttaaagataatatgtttttaggtatacatgagactaatcagtataagttttatacttttgatagtttgtatcctttagctaaaaatggggtttatcaaaaagataattcgtatatgtttagaataagaacgttagattatcaattagctcaatatttatatgatactttagctaaaaatcggactaaagaatttcaaggtctgaccgctgaggttaaaataattaaaccaaaattaatcaaaaaaatatatacacttacaccagtgattttaaaaacagagcagggatactggaaaaacagcattaaaacagaggattttgaaaaacgattaaaaactaatttaattaaaaaatacaaagatataactggtgaagaaattaatgaagattttcaattatattatcaaattaattttaaaaataaggttccggtatcgagaaaatataaaggaattaaattattaggggatatgattgaattagaaattgcagaaaatgataatgctcaaaaattagcttttttagcaatcggatcaggtttgttagaaatgaacgctagaggatttggatttgttaattacatatattattga

SEQ ID NO: 10 - DNA, 1749 bp, Erysipelatoclostridium ramosum
  source 1..1749: mol_type = genomic DNA
ttgaaaggaggttatataatgcttgatgaatgtttacgaatttttaagcaagagcttaaagataatacaaatttagttttaaatacaataattttggctgacggggattatgttttagtacatgatgatggtacttatgatgtagaaaaaattaaatattcaaaaaaagatcattgttttattgaaaaacctgaagatgatctatatgaaaaattatgtttttatgattatcaaagtcaagtaatagatacccaaaaaacttttgatcttcctcataaattaattctttcaaataactatttatcatattttgtaaaaaaagaaaaattgaataataaaaagaaaattaatgatgcaactgaaaaatattttttagcattaagtaatcctagagctaagtataatggtaaagatttagagatgtataattttgtgactcaaaatatcagcgaagttgataaaagaagattattgcaaaataagcagtggttaaaacaatatatttataatttggatggtgttgattataatgaaaaagattatttaaagattttttttgaagcagattatcatcaatatattaatgaaggaaaccgttatcttttgacaaaaatatttaactgtaacgattataacacttatgaaaatgaaacagtgtatggagtaccaaattataatatgcaattaaattctaacaaaccatttttaaaaaatatgacaagaatgcataaagttccatttaaactttccttggaagaagcattgatacaaaaacagttttttgattatttattaaattatgctaataaaggtaaaacaaatatctatattagtagtaatgagaatgatactaaaagaatttattttttagataacaccaaaaaaataaaaaagggatttaatgggctttatcttaaaattaaaaaagacaaagaattaaaaatagagtatatagatacagttactgattataaagaatatttaagtcctatgtttaattttcgaaatgtgattggtgcttttgatgatgaatactatcaagagtataataaaagaaatgaggtagaaaaattacttaatgatgtcttgttttccaagtttttaattaataattattttacggatccagatagtataataagtattaagggtgaaaatattgatgtttataaaaagaatcttattacgtgtagagaagcaatttttgcttggatacatgtaggggcagtgggaaatattaaaaatattttgccaaaagcagcgttagaaacaacgattcactctataaaaatgggacatattaaattggctcaaagacaatttaatctttattttgctataaatgaatattttaataaacaggagaatggtatgaaagatctaagagaaagtattagaacaaaaataaattcccaacagcaaatgaaaatagaaaatgatttagaatatagttttgcagttggacaagcaatagcttatcttcagtcaaagagcaaagctaagaataaaactcaagattttattaatcaatttataactattaagagagatgattctatgaaatcaaaattgaaaaatttatacttacattataattatgctttatctacaggtaagacacgtttcaatatgttgtttgggatgattatggaatatcagagtgatcaaaaaataaatcaaaacatgttgattgctggttatattggagaaaacttaatttatgaaaagggagaaaacgaaaatggataa

SEQ ID NO: 11 - DNA, 918 bp, Erysipelatoclostridium ramosum
  source 1..918: mol_type = genomic DNA
atggataaaagagtatatggagtgattggagtatcatcaaaaatggcaaattggaatgctgatttttctggctatcctagaagtctatctgatggaacgatttttggtagtgacaaagctttaaaatatccaatgagaaaaatgtgggacaatgaaggtaaaaaggtattatatatgaaatctatgaatttttcaaaaaaaaatgatactataacattagtaccaaattcattaaaagagagatatgaaaaattatttgctgttgatgatttaaaaaaatgtaaagataataaggaagtattaactaatttaatgactgctgttgatgttaaaaactttggagcaacatttgcagaagctaaaaataatatttcaattactggtgctgttcagtttgggcagggaattaataaatatggtgatagtgatcttgaaacaatgcaaatactatcaccatttagagatacaaaagatgcagaagcaaaaaattcaactatagggactaaaaaaataatcaatgaagcacattatttctatcctttttcaattaatcctagtgcttataaagaacttgtggctttaggagtcacagatggctacactgaagaagattatttaaattttaaaagaacggctttagtagcagcaacttctttttcatcaaacgctaaagaaggatgtcaaaatgaatttgctttatttgtggaaactaaattggatacatatttaccaaatttaagcgagtatattagttttgaaaaagcagatattaataaaattaaaattgaatgtaacatgttaaatgaattagaagatattttaaatattgaaatttattataatcctgaaacaacagttttagaatctaatttagaaaaagctaaaatctataatttgattactaaaaaagaggtgtag

SEQ ID NO: 12 - DNA, 699 bp, Erysipelatoclostridium ramosum
  source 1..699: mol_type = genomic DNA
atgcaagcacttaagtttactttaagtggtaaaactgctttttttaagcgaccggaagtaaatacgtattattttacttttggtaatattcatagagttgcgctattaggaatatttggggcaattttaggttatcagggctataataatacaattgaactaccagagttttatcaaaaattaaaagatatcaaattatctattttgcctcaaaatgaaaagggttatttttcaaagaaaattcaatattataacaattcagttggttatgcatccaaacaacaaggaggtaatttaattataaaagaacaatggctggaaaatgtttgttgggatattgttgttttgttggataatgaggaggcgaataaattagctaaagctataataaacaaaaaatgtatttttataccctatttaggtaaaaatgatcatccagctaatatcactgatcctgtaattgttgatttacaaaaacgtgatattgataaagaaacgcttgactgcttatttccttcaggaaatttcaaaaaagcagaggatgtatatgttaatttttataaatatcaagaaagtttaccaattggattaaatgaaaaaacgggattgtatgagtatatgacatttattcatacgaacttttatgtggataaaggtgcagtatatagttttaatgataaaaacattgtattttattaa

SEQ ID NO: 13 - DNA, 2508 bp, Erysipelatoclostridium ramosum
  source 1..2508: mol_type = genomic DNA
atggatatcgataaatatctagtttcgctagaaaaaataataattaatattgatcagtgctatggacatagagatgatgatcatcaagaaacaattaatgaacatattcaattatgcacgaaatatttaaaagagatatttaaattaaagaaattggattcaattttaaaatcttttaacatttctttaggagaggggttatcaaatgaaggaaaagaaatgtttaatgagttattttttaacacaattacctttcatgataccggaaaaataaatccgttttttcaaaaaaataaaatgaataatccagtaatgaattatttaaatccgccaaaaaatttaaaatctgatcattcgaaattatcggcatacatctatttaggaacttatttaaataaattgaataggttatcaaggaatgatattggattaataaaaccgattgtttatataaatgcatttgttatttctcgacatcatgctaagattgatgattttagaacatattttatggataaacttgacagtgatgatattttggaaaaaagaattattgaatggatgaacgatgagaaatttataagtcagtttactgagaaattaagtctcgattcattgaatacagatgagtataattatatatttgaaaagctagagaaatatgaaatgaatatacaaatagatctatatgcttatgttagattcttatattcacttctggttttttgtgattattatgcaacaagtgaattttatggtgataataaaaacaatgtaatgcttaaagatgctgattttggtgaaatcattgaactatatgataatagtgaattagttactaagattagaaaaaatgatattggtaaagaaaaaatcaatgcattaagaacaaaagtattcttagaagcggaaagaaatttactagaaaatattgataaaaatattttttatttagaagctccaaccgggagtggtaaaagtaacacagcattaaatcttagtttacgtttaattgaaaatgatgatagtttaaataagataatatatgtatatccttttaatacattagtagaacaaaatctagcatcgttaaataaaatttttggtaatagtaaggctatgaataatattgctgtcgttaattctacaactccattaaaaaaggatgaagatgaaaacttttcggatgaatatcaaaaagcattattagaccgtcaatttctaacatatccaattattctaacgacccacgtaggtctgtttgatactttctttggaaataaccgagaatcagcgtttggtctatgtcaatatgcaaattcggtaatagttttagatgaaatccaaaattatcgaattgaaatatggaatgagatgatcatcttcttaaaagagtttgctcaaattcttaatctaaaaataattatcatgtcagcaacattaccagatttagaattacttacagatgatcgtagtaattcagtttcattaataacaaatcctcaagaatattttttaaatccaatttttaaagatcgagtaaaaacagattatagtctaataaatgtagaaaatactgaagaagcgttattaaatcatgttttagagatgaataagctaaagaaaaaaataatgattgagtttattgttagaaaaagtgcagaaaagttctatcgtcaattaaaagagatagaattagattgtgaagttttatttattagtggttttgatagtgtattagaaagagaaaaaatcattagtactgttaaaacttcaaaacatcttattttagtagcaacccaggtaattgaagccggtgtcgatattgatatggatatcggttataaagatatatctaaacttgatagtgaagaacagtttatgggaagaataaatcgctcatgtaaagaagatggaaaaggaatagtatatttctttaatttaaataaggcaaccaatatttataagggggatgaacgtgttgcacagaaaaatttaacattgtttaatgatgatatgaaaaaagtattgttaaaaaagaactttaattattattaccaaaaacttcttaagactctagcaaatagagctaaaaagcctgaagaaaaaaatgttcccaatttttttagagaaagtgttgcgtgtttagattatcaggcagtaagtaaacgaatggaattgatattagatactagagatcgagtaacgatatttttaggaagaacggtaattaatattgatggggaagaatttgatggtaaagatatttggaatcagtatgtagatttattgaaaaataatgaaatggattatgctaaaaaagtatatgagttgtcagtggttaaaagtatgatgaattattttatgtatcaagttattaaaaaaaatcaatttgattttaaagagcaggttggagatatttattatattgaaaatggtgatgattatattttagatggtaaattaaatacagccctttttgaaactgaggatgagttatttatatag

SEQ ID NO: 14 - DNA, 495 bp, Erysipelatoclostridium ramosum
  source 1..495: mol_type = genomic DNA
atggaagtaaatggaacattaataaactattattttcattgtcaaaggcaatgttatcttcatggtaatcgacttaatttagaggatgaaagtgaaaatgtgaagattggtaaagcattgcatgaagaaaagtataaagatgataagaattcagagattgcaattgataatattaggttagataaattaactgcaaaatatttaactgaaacaaaaaagtctgatgctgatttagttgcagcaaagtggcaactattatattatcttaaaattttaaagtcaaaaggaataatacgaaaaggacgaatagaagtaattgaaaagaataaacaaaataaatcttttattgaagtggagctaacagaaattgaagaaaaagaattagataaaattgttattaaaattaaagaattattagaaaatgatcaaataccaccagttttaaatgaaaataaatgtaaaaaatgtgcatattatgcatattgttatatatag

SEQ ID NO: 15 - DNA, 999 bp, Erysipelatoclostridium ramosum
  source 1..999: mol_type = genomic DNA
atgaaaagtactaaatacataactagtatgggagatttaaaacgtaaagatgattcgctttgttttagaaaaagtggtaagaatatatatttaccagtagaaaatataaaagagatatattgtttaaatgaaatttctcttaatactaaattattagattttatctcatctaaaaatataattattcatttttttaactattatcagggttatagtggtaccttttatccaaaagataaatatactagtggtaaattgttgattaaacaggttgaaacatttaataattatcgtattgatattgcaaaagcaatagttaatggtattgcaattaacattgatgaaactttatatcactattataagcatggtaaaaatgaggttaaagaaacaatagactggttaagaaaagaggtgccaaaacgtttaaaaaaagcagaagatattaaaatgattatggcggttgaaggagaaatatggcaacgtttttatagtatgtttatatatatattacctgaagattttataatgaacaaaagagtaaagagaccacctgataatccaattaatgcactaatttcttttggtaacactttactttatacaaaaacaatttcagcgatatatcaaactcacttaaatcaaagtatttcttttttacatgaacctacagaacaaagattttctttgtctttagatttaagtgaagtatttaaaccgatcattgtttttaaaacaatctttgaattagtcaatactaaaagattaacggttgagaaacattttgataagaaaactaattattgtcttttaaatgataaaggtagggatatatttattgaagcttttgaaaatagaatggaaactaaatttttacatactaaattaaagagaaagataacatataaaacagctattaaatatgatggttataaattaattaaaacaatttttgaaaataaattatttataccattttctataaaggataaatattaa

SEQ ID NO: 16 - DNA, 288 bp, Erysipelatoclostridium ramosum
  source 1..288: mol_type = genomic DNA
atgaaaataaaaaatcataactatgtaatcgtctgttacgatattggggaaaaaagagtaaataaaatatttaagatatgtaaaaaatatcttcctcattatcaatattctatttttaagggtcctattaccccttctaaattaattttattaaaaaaagaactaaaaaaagctataaacaaagaagaagattgtgtaagtattattaaattacagagtgaggattcatttgatgaagaaattttagggagtcaaaaagagggaaatgaagatagtttaattatataa

SEQ ID NO: 17 - DNA, 30 bp, Erysipelatoclostridium ramosum
  source 1..30: mol_type = genomic DNA
gtttaataacaacataagatgtattgaaat

SEQ ID NO: 18 - DNA, 30 bp, Erysipelatoclostridium ramosum
  source 1..30: mol_type = genomic DNA
gtttaataacaacgagatattaaagtgaaa

SEQ ID NO: 19 - protein, 218 aa, Clostridium sp.
MKEVQICQVIYKDIKVNRSTAPMIRGYFANKYNEIEQMHNHKDDKFIYRYPHIQYKVLNNNPVLLGIEDGAKIIQEMTFFLDDVIKIGQKEFESNQREISFKKVKLGVSDKIIKYKFITPWLALNQNNIKKYTAADLIEKEEILKRVLIGNILSFSKGMDYTVDETIKVKLDLNQIDVKFKQKEMLAFIGEFFINFEIPDNFAIGKGISRGFGTVKKI

SEQ ID NO: 20 - protein, 337 aa, Clostridium sp.
MEIDKSYLDEVWVNIIEEVQKDANQSYFIIITKNNKKPRELFTQKYEQKIKETYLISDTKLNKSLKQSKCHLCGQRKKTYDTAIYKCFTNDKEIYKNTDKYNFSICEDCLLDVLNGRNYINDILKISWIGSEVMFLPHEFNVYIKEIYEEIDNDKHITSLLAQIKEAEEEVLDEISKTNCMTDIIFFSDPKSSSEWKITYAIRDVMPSRMSIISRNISKYKNKKIDAYFCLAKIMNYLCYSDGKFDSKNKDRMRLLDIIFHGSKYSRELFFNKVMSKYKTKYFESLNNKKATNRKYIIKDIHEIYNFMCNCGCLENSWMMINEEGEIMKYNNISEFF

SEQ ID NO: 21 - protein, 292 aa, Clostridium sp.
MSNTKSREFLLIWDSTMSNPNGDMLNENRPRQDEITGQLEASDVRVKRFAREEWINKGENVLVQTVEEKGKIQTCQKRVETIQKSAKIKDDDIEKYLTDNYIDVKLFGAVITKPKRDILGPLQVAWSKSVHEAQIKFMQGNAAYASSEGKSQASIWTKYISPYALFKTYAVYNNLVAKKQNINVEDKDIESFIEALIDGMKHYRSTSKNQMPRLLVEVIYNNSKLDGELDYIDIIKDVEDEELRNIDQVQIDLSKLSRYYDLKKDDIEKIVIYTHPSVKLLNIDDRFELKTI

SEQ ID NO: 22 - protein, 224 aa, Clostridium sp.
MKCLVFKVRGDYARFRKSYTTTSALTYLLIHPVAVRGLIGAMLGIDREKLYEKTKDIEVAIQVVNQVRKDMQSFNLVNMKPNDKIFRFPSNVEFLRNVEYRIFIKAEDKTLYEIDNCIKQGEYIFTPYLGSSEHIAKIEYEGIYNCEKLNSGNYKVISAVSLEENKIYFGNGNIILMTDNIPTRRDIDREYIEYKKVIFATNENTINVETENCYKVGGYNVIFI

SEQ ID NO: 23 - protein, 757 aa, Clostridium sp.
MSYSYKLKSHPTQSLYDHLTGVRDIALKTHKHHTIKPEIDDFIEIVCMCHDFGKGTTYFQRYIENDFRGIEKDHGPISAMFTYWMLPDKWKHLGFLIVKKHHGDINNASDECRIDEVSWDFKKQIKDILDNTIDELNQIYDKYLEDKNIEAFLNWLDDESNLKSIKKEFRKKKYNIEDLLLCEYVYSLLLTGDKSQLIRNDAYIPDKQYPLSFIENYKADLVKDALIKNPKLKESDVFNLRNEIYDDMINKLDTIDFDKENIFSINVPTGTGKTILAYSAAFYICNKIIKNNLNIRPHIIYSLPFTSVIDQNYEVLKEIVENNINKEISSEDLMKFHSVVPIEYENFEGYDARFCFENWQSKIISTTFVQLLNTIFKIGKNSIVNRFHRLANSVIILDEVQAIDEKYYKIISEFISIMVRQYNCYIILVTATMPMLLDTIDLIEDKEKYFRKLNRIEIINNSESEITLDEFEDIVLEDICENKDKSFLIVLNTVKSSKNIYKYLKENTDRDIMYLSTEIYPKLRLEKINKIKNSDKKYVVVSTQLIEAGVDIDMDIVYRDFSTLDSINQTAGRANRNGVGGKGIVKLYKIVDNDRRICNYIYPRYLLNATEEVLDGKDIIEEKDIYDCNKEYFLKVKNRLSNDTSDELLDLIPKLQFKKFRDKFELIENDEFRRVDIIVNADNITSSIIQQLENDNEIDNIDLKNKFRILRQYIVSVPRKDMSEINDIGLKKYNITYINKEDYSEEEGIIRQKQVIW

SEQ ID NO: 24 - protein, 203 aa, Clostridium sp.
MKSSQSFFNEFDYYITPSELIEFNYCKRFTYYMKCLNVPQFEENRFKVQKGKEIHSKKENENKQYLRKKLGVIDKKINVDLYSEKYKIKGIVDEVLTLSDNTMAPLDYKFAKFDDVLYKTYKNQMLMYSIMIEEMYNCKVNKCFLVYTRSSNLIKEFNVEEKELKKLKKDIRDYFKVMEGYFPKATSSKARCIDCCYRNICIK

SEQ ID NO: 25 - protein, 328 aa, Clostridium sp.
MKLVVNSEGVYLTKVGECFCLKKEGQKQEIAAKKVEQIIITTSTALSTDAIELAVENNIDIVFLKKYGKPFGRVWHSKVGSISTIRKRQLNLDSNKMGTILVKEFLTQKMTNQVEFLNELSMNRRDNRKIEIKEDVEKIKGLIEKIKSISEESKINDIRGSLQGYEGTASRIYFSRLSDLMPEKYKFNGRSKNPAKDEFNSMLNYGYGIMYSNVERACILAGLDPYIGIMHVDNYNRQAFVFDLIEMYRIYIDKIVFKLFTTKKVKKEHFDKVEGGLYLNKAGKEILISKYNEEIEKKIQYKGRKIELQNIIQYDCHNIANKILKEGD

SEQ ID NO: 26 - protein, 96 aa, Clostridium sp.
MLVWVVYDISSNKVRRNAIKACKNVGLYRVQKSVFLGEIEENDFDELKLKMEDIIDLTSDSVYIFPMTKKGLNQAGLIGQVFDKELVTDEIISKFF

SEQ ID NO: 27 - DNA, 657 bp, Clostridium sp.
  source 1..657: mol_type = genomic DNA
atgaaagaagttcaaatatgtcaggtaatatataaagatataaaagtaaatagatctacagcacctatgataagaggatattttgcaaataaatacaatgaaatagaacaaatgcataatcataaagatgataaatttatatatagataccctcatattcaatataaagtactaaataataatcccgtattgttgggaatagaagatggtgcaaaaataattcaagaaatgacattttttttagatgatgtaataaaaatcggtcaaaaagaatttgaatctaaccaaagagaaatatcttttaaaaaagttaaattaggagttagtgataaaataataaaatataaatttataactccatggttagctttaaatcaaaataatattaaaaaatatacagcagcagatctaatagaaaaagaagaaatactaaaaagagtacttataggaaatattttgtctttttcaaaaggaatggattatacagttgacgaaactataaaagtaaagttagacttaaatcaaatagatgttaaatttaaacaaaaagaaatgttagcttttataggagaattttttataaactttgaaataccagataattttgcgataggtaaaggaatatcgagaggatttggaactgtaaaaaagatttaa

SEQ ID NO: 28 - DNA, 1014 bp, Clostridium sp.
  source 1..1014: mol_type = genomic DNA
atggaaatagacaaaagttatctagatgaagtttgggtaaatataatcgaggaagttcaaaaagatgcaaatcaatcgtattttattataatcacaaaaaataacaaaaaacctagagaattattcacacaaaaatatgaacaaaaaataaaagagacatatctaatatcagacactaaattaaataaatccctaaaacaaagtaagtgccacttatgtggtcaaagaaaaaaaacttatgacacagcgatatataaatgttttacaaatgataaagaaatttataaaaatacagataaatataacttctctatatgtgaagattgcttattagatgtattaaacggaagaaactatatcaatgatattctaaaaataagctggattggttctgaagtgatgtttttgccacatgaatttaatgtatatataaaagaaatatatgaagaaatagataatgataaacatataacatctttactcgctcaaataaaagaagctgaggaagaagtacttgatgaaatttcaaagacaaattgtatgacagatataatatttttctcagatcctaaaagtagttctgagtggaagattacttatgcaataagagatgtaatgccttctagaatgagtataatttcaagaaatataagcaaatataaaaataagaaaatagatgcatatttttgtttagctaaaattatgaattatctatgttatagcgatggaaagtttgattctaagaataaagatagaatgagattgttagatattatttttcatggaagtaaatatagtagagaactatttttcaacaaagttatgtcgaaatacaaaacaaaatactttgaaagcttaaacaataaaaaggctacaaacagaaaatacataataaaagatatacacgaaatatataactttatgtgtaattgtgggtgtttagaaaattcatggatgatgataaatgaggagggtgaaattatgaaatacaataatataagtgagtttttttga

SEQ ID NO: 29 - DNA, 879 bp, Clostridium sp.
  source 1..879: mol_type = genomic DNA
atgtcaaacacaaaatcaagagaatttttattaatatgggattcaacaatgtcaaatccaaatggagatatgttaaatgaaaacagaccacgtcaagatgagattacaggtcaattagaagcatctgatgtcagagtaaaacgttttgcaagagaagaatggataaataaaggtgaaaatgtattagttcaaacagttgaagaaaaaggtaaaatacaaacttgtcaaaaaagagttgaaactattcaaaaatctgcaaaaataaaagatgacgacattgaaaaatatttaactgataattatatagatgttaaattatttggggcagttataacaaagccaaaaagagatatattaggtcctcttcaagttgcatggagcaaatctgtacatgaagctcaaataaagtttatgcaaggtaatgcagcatatgcgagttcagaaggaaaatcacaagcatctatttggacaaagtatatatctccatatgcactatttaaaacatatgctgtttataataacttagtagcaaaaaaacaaaatataaatgtagaagataaagatatagaatcatttatagaagcacttatagatggtatgaaacattatagaagtacatctaaaaatcaaatgcctagattattagtagaagtaatttataataatagcaaattagatggagaactagattatattgatataataaaagatgttgaagatgaagaacttagaaatatagaccaagttcaaatagatttatcaaaattatcaagatattatgatttgaagaaagatgatatagaaaaaatagttatttatacacatccaagtgtaaaactattaaatatagacgatagatttgaacttaaaactatatag

SEQ ID NO: 30 - DNA, 675 bp, Clostridium sp.
  source 1..675: mol_type = genomic DNA
atgaaatgtttagtttttaaagttagaggtgattatgcaagatttagaaaatcatatacaacaacctcagctctaacttatttattaattcatccagtagctgttagagggttaataggtgctatgcttgggattgatagagaaaaactctatgaaaaaacaaaagatattgaagtcgctattcaagttgtaaatcaagttagaaaagatatgcaatcttttaatttagtaaatatgaaagctaatgataaaattttcagatttccatcaaatgtagagtttttgagaaatgtagaatatagaatatttataaaagcagaggataaaactttatatgaaattgataattgcataaaacaaggggaatatatatttactccatacttaggttcatctgaacatatagccaaaatagagtatgaaggtatttataactgtgaaaaattaaatgcaggaaattataaagtaattagtgcagtatccttagaagaaaataaaatatattttggagatagtgatataatacttatgactgataatattccaacaagaagggatatagatagagaatatatagaatataaaaaagtgatatttgctacaaatgaaaatactataaatgtaggtacagaaaattgctataaagtaggtggatataatgtcatattcatataa

SEQ ID NO: 31 - DNA, 2274 bp, Clostridium sp.
  source 1..2274: mol_type = genomic DNA
atgtcatattcatataaattaaaatcacatcctacccaaagcttatatgatcatcttacaggggttagagatatagcattaaaaacacataaacaccatactataaaacctgaaatagatgattttatagaaattgtatgtatgtgccatgactttggaaagggaactacatattttcagagatatattgaaaatgattttagaggaatagaaaaggatcatggacctatttcagctatgtttacatattggatgcttccagataagtggaagcatctagggtttcttatagtgaaaaaacaccacggagatataaataatgcaagtgatgagtgcagaattgatgaggtatcttgggattttaaaaaacagataaaagatatattggataatactatagatgaattaaatcaaatatatgataaatacttagaagataaaaatatagaagcatttttaaattggttggatgatgagtctaatttaaaaagtataaaaaaagaatttagaaagaaaaaatataatatagaagatttacttttatgtgaatatgtatattctcttcttttaacaggagataaaagtcaacttatacgaaatgatgcctatataccagataaacaatatccactttcttttattgaaaattataaggcagatttagttaaagatgcgcttattaaaaatccaaagttaaaagaatcagatgtgttcaatttaagaaatgaaatatatgatgatatgataaataaattagatactatagactttgataaagaaaatattttttctataaatgttccaacaggaacaggcaaaactattttggcttattctgcagctttttatatttgtaataagataataaaaaataatttaaatattagacctcatattatatattcattaccatttacatctgtaatagatcaaaattatgaggtgttaaaagagatagttgaaaacaatataaataaagaaatatcatctgaagacttgatgaagtttcatagtgttgtgcctatagagtatgaaaactttgaaggatatgatgcacgtttttgttttgaaaattggcaaagtaaaataatatcaacgacatttgtacagcttttaaatactatatttaaaattggaaaaaactcaatagtaaatagatttcatagacttgcaaattcagttataattttggacgaggttcaagcaattgacgaaaaatattataaaataatttcagagtttataagtataatggtcagacagtacaattgttatattatattagttactgctacaatgccgatgcttttagatactatagatcttattgaagataaggaaaaatactttagaaaactaaatagaattgagataattaataattcagaaagtgaaataactttagatgaatttgaagatattgttttagaggatatttgtgaaaataaagataaaagctttcttattgttttaaatactgtaaaaagttctaaaaatatttataagtacttaaaagaaaatacagatagagatattatgtatctttctacagagatttatccaaagttaagactagaaaaaataaataaaataaaaaatagcgataaaaaatacgtagttgtatcaactcaacttatagaagctggcgtagatatagacatggatatagtatatagagatttttcaacattagattcaataaaccaaacagcaggaagggcaaatagaaatggtgttggaggaaaaggaatagtaaaactttataaaatagtagataatgatagaagaatatgcaattatatttatccaagatatctactaaatgcaacagaagaagttttagatggaaaagatattatcgaagaaaaagatatttatgactgtaataaagagtattttttaaaagtaaaaaatagactatcaaatgatacatcagatgaacttttagacttgattccaaaattacaatttaaaaagtttagagacaagtttgaacttatagaaaatgatgaatttagacgagtggacataatagtaaatgctgataatattacgagttctataatacaacaattagaaaatgataatgaaatagataatattgatttaaaaaataagtttaggatattaagacaatatatagtatctgttcctagaaaagatatgtctgaaattaatgatatagggttgaaaaaatataatataacatatataaataaagaagattattcagaagaagaaggtattattagacaaaaacaagtaatctggtag

SEQ ID NO: 32 - DNA, 612 bp, Clostridium sp.
  source 1..612: mol_type = genomic DNA
atgaaatcatctcaaagtttttttgatgaattcgattactatataacaccttctgagcttatagaatttaattattgtaaaagatttacttattatatgaaatgcttgaacgtaccacaatttgaagaaaatagatttaaagtacaaaaaggaaaagaaatacattctaaaaaagaaaatgaaaataaacagtatcttcgtaaaaaattaggtgttatagataaaaaaataaatgttgatttatattctgaaaaatataaaattaagggtatagtagatgaagttttaacattaagtgataatactatggcacctttggattataaatttgctaaatttgatgatgtactatataaaacatataaaaatcaaatgttgatgtactctattatgattgaagaaatgtataactgcaaagtaaataaatgctttttagtttatacaagaagctcaaatttaataaaagaatttaatgtagaagaaaaagaattaaaaaagctaaaaaaagatataagagattattttaaagtgatggaaggatattttccaaaagcgacatcatcaaaagctagatgtatagattgttgctatagaaatatatgcataaaataa

SEQ ID NO: 33 - DNA, 987 bp, Clostridium sp.
  source 1..987: mol_type = genomic DNA
atgaagctagttgttaattcggaaggagtatatttgactaaagtaggcgaatgtttttgtttgaaaaaagaggggcagaaacaagaaattgctgctaagaaggttgaacaaataattataacgacatctacagctttgtctactgatgctattgagttagctgttgagaataatattgatatagtgtttttaaaaaagtatggaaaaccttttggaagagtatggcattctaaagttggtagcataagtactattagaaaaagacagttaaatttagattctaataaaatgggtacaattttagtaaaagagtttttgactcaaaagatgactaatcaggttgaatttttaaatgaattatctatgaatagaagagataatagaaaaattgaaataaaagaagatgtagaaaaaataaaggggttaatagaaaaaattaaatctatatcagaagaatcaaaaataaatgatataagaggaagtttacaaggatatgaagggactgcaagcagaatatatttctctagattatcagatttaatgccagaaaaatataagtttaatggacgtagcaaaaatccagctaaagatgaattcaactgtatgttaaattacggatatgggataatgtattctaatgtagagagagcatgtatattagcggggttagatccttatataggaataatgcatgttgataattataatagacaagcttttgtatttgacttaattgaaatgtataggatttatattgataaaattgtatttaaactttttacaactaagaaagttaaaaaagaacattttgataaggttgaaggaggattatatctaaataaagctggaaaagaaatattaatttccaagtataacgaagaaatagaaaagaaaattcaatataaaggtagaaagatagaattacaaaatataattcaatatgactgtcataatatagccaataaaattttaaaggagggagattga

SEQ ID NO: 34 - DNA, 291 bp, Clostridium sp.
  source 1..291: mol_type = genomic DNA
ttgttagtctgggtagtatacgatatttcgagtaataaggttagacgaaatgctattaaagcgtgtaaaaatgttggactatacagagtacaaaaaagcgtgtttttaggtgaaatagaagaaaatgatttcgatgaattgaagctaaaaatggaagatataatagatttgactagtgattcagtatatatattcccaatgactaaaaaaggactgaatcaagcaggattaattggacaagtttttgataaggagttggttactgatgaaatcatctcaaagtttttttga

SEQ ID NO: 35 - DNA, 32 bp, Clostridium sp.
  source 1..32: mol_type = genomic DNA
gtttcaatccacgcacccgtgcagggtgcgac

SEQ ID NO: 36 - DNA, 30 bp, Clostridium sp.
  source 1..30: mol_type = genomic DNA
gtttcaattccaacatggtacgattaaagc

SEQ ID NO: 37 - DNA, 30 bp, Clostridium sp.
  source 1..30: mol_type = genomic DNA
gtttcaattccaatatggcacgattaaagc

SEQ ID NO: 38 - DNA, 30 bp, Clostridium sp.
  source 1..30: mol_type = genomic DNA
tgctttattacaatgtggtaagagtaaagc

SEQ ID NO: 39 - DNA, 29 bp, Clostridium sp.
  source 1..29: mol_type = genomic DNA
tttcaattccaacttggtacgattaaaac

SEQ ID NO: 40 - DNA, 30 bp, Clostridium sp.
  source 1..30: mol_type = genomic DNA
aacgcaattccaacttggtacgattaaaac

SEQ ID NO: 41 - DNA, 30 bp, Clostridium sp.
  source 1..30: mol_type = genomic DNA
gtttcaattccaacttggtacgattaaaac

SEQ ID NO: 42 - DNA, 30 bp, Clostridium sp.
  source 1..30: mol_type = genomic DNA
gttgcaattccaatatggtaagattaaagc

SEQ ID NO: 43 - DNA, 27 bp, Clostridium sp.
  source 1..27: mol_type = genomic DNA
tcaattccaatatggtacgattaaaac

SEQ ID NO: 44 - DNA, 310 bp, Clostridium sp.
  source 1..310: mol_type = genomic DNA
aattaatttttatataggaggtgagtttatgagttgtatcacaatgagttttaaatgcctgttctgttccaaacgttcaggctgtccaattgcattttaattgaaggaggttattatatgggtattgtaaaattaaatagtttttataggcattgtgcaatatgtggtttgtgcagtgtctttttttatgtataaattgattaatttaaaatgaggaatacagattaaaaaaagacaaagaatttagttgaatttgatggctttttttttgagggtaatgtctaaaaaaatattataaaaagttggtact

SEQ ID NO: 45 - DNA, 269 bp, Clostridium sp.
  source 1..269: mol_type = genomic DNA
tttttcaatattgtagactttttggtgtatttagtataaaatctaataaagtaattaaatatcttatctaatattgaaatattctaacctttaaatttaaagtttttgcggtattttagcttattttttaatccaactgccacgcaccgctgataccaacggtttgaggtacttttaaggacgtaaagtttttcttatatgtaagaaaaaagttgtaccccgcaacaggggtctgaaagctagtatttgcaagggctgaaaatttaggg

SEQ ID NO: 46 - DNA, 303 bp, Clostridium sp.
  source 1..303: mol_type = genomic DNA
ataaaattaaacttccccgaaattttatttttggggagttatttttatttttactgtttatgacacttctttcaactacataaaaaatctttatctttagagttatttatgtaaatcactccttattatacatataaatttatacaaataataacataaattgtaaaattattattaatgttcatatcttaaaaatctattttttatctttaaaacatcttacatattttagcaaatttcattatgtaaaacaccctatttaataaaacaatttaaacagagtagaaaaatagtaaaactttt

SEQ ID NO: 47 - DNA, 186 bp, Clostridium sp.
  source 1..186: mol_type = genomic DNA
ggaggctgcagtgcatatggatatttctgtggagtttgtagagacttgcttgtatgagataattgtaagaaaaaaagagagttagattttttaactctcttttgaaatttcaacttcaaaataccaaagaaaaactttttgaatctcttttcatattaagtagtttagtttctatactattttatt

SEQ ID NO: 48 - DNA, 205 bp, Clostridium sp.
  source 1..205: mol_type = genomic DNA
aaaagatttttaatagttttgttcctacaaaattattggaaattttgaactttgacaattgaatgtgttggaattacttggtttgagatagatgttaataagctaaaaaatgcattttttttaatgggaaatcattgttttgaggttagatgaaaataaagcaacacattgaaaatcagtaattccaatggttttaggttctacg

SEQ ID NO: 49 - DNA, 147 bp, Clostridium sp.
  source 1..147: mol_type = genomic DNA
ataatcacctcctgcaaagaatattaataattacatatattaataaaaccttatatttagataatagtcctattgttattatatgtcaataaatgtttttaaattttaatccttgttttattggaattttataattacatcgattaa

SEQ ID NO: 50 - DNA, 174 bp, Erysipelatoclostridium ramosum
  source 1..174: mol_type = genomic DNA
tttaccaggctcaatttctttttaatgtcctaaaactgttgggagagtaagcgtttaagagaattttactagcaattataaaaaagtaaaatcatttggtaaaaaagtagaaaagtattgatagatataggttttgaggtataatagaaagtgtaaaatggcttatttactagg

SEQ ID NO: 51 - DNA, 260 bp, Clostridium sp.
  source 1..260: mol_type = genomic DNA
tccagataaattaaacttatataattaaatccaggctggttatataattatataatcatcctgtaatataaaaattaaaaataatattataaaaagtaaacaaaaagacaacaaaaaacaactaatcgagaaaaataataacaaaattaaaattaaaattaaaatacatagaaatttatgaacttacccatcaatttgtttgtgatattttatgttttttacaaactatgacttgattagttcaatggaaggagaataac

SEQ ID NO: 52 - DNA, 84 bp, Erysipelatoclostridium ramosum
  source 1..84: mol_type = genomic DNA
tatccaatcaaaatcttattgattttttcaactaaataaattagaatgtatatgtacaaaaatataaactaggaggaaccttaa

SEQ ID NO: 53 - DNA, 42 bp, Clostridium sp.
  source 1..42: mol_type = genomic DNA
aaaagatcccggattctgtatgatgcagagtccgggattttt

SEQ ID NO: 54 - DNA, 31 bp, Clostridium sp.
  source 1..31: mol_type = genomic DNA
ggaacccctggccaatatggtcaggggttct

SEQ ID NO: 55 - DNA, 45 bp, Clostridium sp.
  source 1..45: mol_type = genomic DNA
atgaattgcagaaatgcatttcagatatttttgaaccttgaaaac

SEQ ID NO: 56 - DNA, 31 bp, Clostridium sp.
  source 1..31: mol_type = genomic DNA
cccctatttttgtgcaatatgtagaaaaata

SEQ ID NO: 57 - DNA, 39 bp, synthetic construct
  misc_feature 1..39: note = rho independent terminator
  source 1..39: mol_type = other DNA
aaaaaaaaaccccgcccctgacagggcggggtttttttt

SEQ ID NO: 58 - DNA, 39 bp, Lactobacillus crispatus
  source 1..39: mol_type = genomic DNA
caaaaaaagcatgagaattaattttctcatgcttttttg

SEQ ID NO: 59 - DNA, 38 bp, Lactobacillus crispatus
  source 1..38: mol_type = genomic DNA
aaaaaagatgcacttcttcacaggagcgcatctttttt

SEQ ID NO: 60 - DNA, 32 bp, Lactobacillus crispatus
  source 1..32: mol_type = genomic DNA
caaaaagagcggctataggccgctttttttgc

SEQ ID NO: 61 - DNA, 37 bp, Lactobacillus crispatus
  source 1..37: mol_type = genomic DNA
gtaaaaatggcttgcgtgttgcaagccatttttttac

SEQ ID NO: 62 - DNA, 54 bp, synthetic construct
  source 1..54: mol_type = other DNA
atgctaatggagtttactacaagttaatccggcaaagctaaatggccggcccgt

SEQ ID NO: 63 - DNA, 10 bp, Clostridium sp.
  source 1..10: mol_type = other DNA
ttacaattta

SEQ ID NO: 64 - DNA, 10 bp, Clostridium sp.
  source 1..10: mol_type = other DNA
gtttttataa

SEQ ID NO: 65 - DNA, 10 bp, Clostridium sp.
  source 1..10: mol_type = other DNA
atcccattta

SEQ ID NO: 66 - DNA, 10 bp, Clostridium sp.
  source 1..10: mol_type = other DNA
cattttcttt

SEQ ID NO: 67 - DNA, 10 bp, Clostridium sp.
  source 1..10: mol_type = other DNA
accattttta

SEQ ID NO: 68 - DNA, 10 bp, Clostridium sp.
  source 1..10: mol_type = other DNA
aatttagcta

SEQ ID NO: 69 - DNA, 10 bp, Clostridium sp.
  source 1..10: mol_type = other DNA
ttctttttta

SEQ ID NO: 70 - DNA, 10 bp, Clostridium sp.
  source 1..10: mol_type = other DNA
ataatctgta

SEQ ID NO: 71 - DNA, 10 bp, Clostridium sp.
  source 1..10: mol_type = other DNA
tgaaagttta

SEQ ID NO: 72 - DNA, 10 bp, Clostridium sp.
  source 1..10: mol_type = other DNA
accgagtcgg

SEQ ID NO: 73 - DNA, 10 bp, Clostridium sp.
  source 1..10: mol_type = other DNA
actttattta

SEQ ID NO: 74 - DNA, 10 bp, Clostridium sp.
  source 1..10: mol_type = other DNA
tatatattct

SEQ ID NO: 75 - DNA, 10 bp, Clostridium sp.
  source 1..10: mol_type = other DNA
gtattattta

SEQ ID NO: 76 - DNA, 10 bp, Clostridium sp.
  source 1..10: mol_type = other DNA
cccagaaggt

SEQ ID NO: 77 - DNA, 10 bp, Clostridium sp.
  source 1..10: mol_type = other DNA
gtaacattta

SEQ ID NO: 78 - DNA, 10 bp, Clostridium sp.
  source 1..10: mol_type = other DNA
ttataattaa

SEQ ID NO: 79 - DNA, 10 bp, Clostridium sp.
  source 1..10: mol_type = other DNA
tcttctttta

SEQ ID NO: 80 - DNA, 10 bp, Clostridium sp.
  source 1..10: mol_type = other DNA
tttacagcca

SEQ ID NO: 81 - DNA, 10 bp, Clostridium sp.
  source 1..10: mol_type = other DNA
tttagtttta

SEQ ID NO: 82 - DNA, 10 bp, Clostridium sp.
  source 1..10: mol_type = other DNA
cagattgttc

SEQ ID NO: 83 - DNA, 10 bp, Clostridium sp.
  source 1..10: mol_type = other DNA
cgattattta

SEQ ID NO: 84 - DNA, 10 bp, Clostridium sp.
  source 1..10: mol_type = other DNA
taactgagat

SEQ ID NO: 85 - DNA, 10 bp, Clostridium sp.
  source 1..10: mol_type = other DNA
agtcatttta

SEQ ID NO: 86 - DNA, 10 bp, Clostridium sp.
  source 1..10: mol_type = other DNA
ttacagctat

SEQ ID NO: 87 - DNA, 10 bp, Clostridium sp.
  source 1..10: mol_type = other DNA
attttcttta

SEQ ID NO: 88 - DNA, 10 bp, Clostridium sp.
  source 1..10: mol_type = other DNA
ccaatatcta

SEQ ID NO: 89 - DNA, 10 bp, Clostridium sp.
  source 1..10: mol_type = other DNA
tctgctttta

SEQ ID NO: 90 - DNA, 10 bp, Clostridium sp.
  source 1..10: mol_type = other DNA
ttatcagtta

SEQ ID NO: 91 - DNA, 10 bp, Clostridium sp.
  source 1..10: mol_type = other DNA
gagcttttta

SEQ ID NO: 92 - DNA, 10 bp, Clostridium sp.
  source 1..10: mol_type = other DNA
actctctatt

SEQ ID NO: 93 - DNA, 10 bp, Clostridium sp.
  source 1..10: mol_type = other DNA
cactccttta

SEQ ID NO: 94 - DNA, 10 bp, Clostridium sp.
  source 1..10: mol_type = other DNA
ttttgcaaca

SEQ ID NO: 95 - DNA, 10 bp, Clostridium sp.
  source 1..10: mol_type = other DNA
ctggtgttta

SEQ ID NO: 96 - DNA, 10 bp, Clostridium sp.
  source 1..10: mol_type = other DNA
aactttcact

SEQ ID NO: 97 - DNA, 10 bp, Clostridium sp.
  source 1..10: mol_type = other DNA
catctgtaag

SEQ ID NO: 98 - DNA, 10 bp, Clostridium sp.
  source 1..10: mol_type = other DNA
aaagttttgc

SEQ ID NO: 99 - DNA, 10 bp, Clostridium sp.
  source 1..10: mol_type = other DNA
tttattttta

SEQ ID NO: 100 - DNA, 10 bp, Clostridium sp.
  source 1..10: mol_type = other DNA
aagcgttttg

SEQ ID NO: 101 - DNA, 10 bp, Clostridium sp.
  source 1..10: mol_type = other DNA
aattcattta

SEQ ID NO: 102 - DNA, 10 bp, Clostridium sp.
  source 1..10: mol_type = other DNA
ctcctacatc

SEQ ID NO: 103 - DNA, 10 bp, Clostridium sp.
  source 1..10: mol_type = other DNA
aaggtcttta

SEQ ID NO: 104 - DNA, 10 bp, Clostridium sp.
  source 1..10: mol_type = other DNA
ttgaattttt

SEQ ID NO: 105 - DNA, 10 bp, Clostridium sp.
  source 1..10: mol_type = other DNA
tagattttta

SEQ ID NO: 106 - DNA, 10 bp, Clostridium sp.
  source 1..10: mol_type = other DNA
tattgttttt

SEQ ID NO: 107 - DNA, 10 bp, Clostridium sp.
  source 1..10: mol_type = other DNA
tttgcattta

SEQ ID NO: 108 - DNA, 10 bp, Clostridium sp.
  source 1..10: mol_type = other DNA
ccactgaatt

SEQ ID NO: 109 - DNA, 10 bp, Clostridium sp.
  source 1..10: mol_type = other DNA
aaatgcttta

SEQ ID NO: 110 - DNA, 10 bp, Clostridium sp.
  source 1..10: mol_type = other DNA
atccttttga

SEQ ID NO: 111 - DNA, 10 bp, Clostridium sp.
  source 1..10: mol_type = other DNA
agattgttta

SEQ ID NO: 112 - DNA, 10 bp, Clostridium sp.
  source 1..10: mol_type = other DNA
aagaatgcaa

SEQ ID NO: 113 - DNA, 10 bp, Clostridium sp.
  source 1..10: mol_type = other DNA
agttttttta

SEQ ID NO: 114 - DNA, 10 bp, Clostridium sp.
  source 1..10: mol_type = other DNA
ttatagcaaa

SEQ ID NO: 115 - DNA, 10 bp, Clostridium sp.
  source 1..10: mol_type = other DNA
atgattttta

SEQ ID NO: 116 - DNA, 10 bp, Clostridium sp.
  source 1..10: mol_type = other DNA
atacaagaat

SEQ ID NO: 117 - DNA, 10 bp, Clostridium sp.
  source 1..10: mol_type = other DNA
gaggtgttta

SEQ ID NO: 118 - DNA, 10 bp, Clostridium sp.
  source 1..10: mol_type = other DNA
aatgcattag

SEQ ID NO: 119 - DNA, 10 bp, Erysipelatoclostridium ramosum
  source 1..10: mol_type = other DNA
attataatca

SEQ ID NO: 120 - DNA, 10 bp, Erysipelatoclostridium ramosum
  source 1..10: mol_type = other DNA
tggaggcgtt

SEQ ID NO: 121 - DNA, 10 bp, Erysipelatoclostridium ramosum
  source 1..10: mol_type = other DNA
accttcatcg

SEQ ID NO: 122 - DNA, 10 bp, Erysipelatoclostridium ramosum
  source 1..10: mol_type = other DNA
tgaattttca

SEQ ID NO: 123 - DNA, 10 bp, Erysipelatoclostridium ramosum
  source 1..10: mol_type = other DNA
agcattatca

SEQ ID NO: 124 - DNA, 10 bp, Erysipelatoclostridium ramosum
  source 1..10: mol_type = other DNA
gtaaatcgtt

SEQ ID NO: 125 - DNA, 10 bp, Erysipelatoclostridium ramosum
  source 1..10: mol_type = other DNA
ttcaagatca

SEQ ID NO: 126 - DNA, 10 bp, Erysipelatoclostridium ramosum
  source 1..10: mol_type = other DNA
cttaaatctc

SEQ ID NO: 127 - DNA, 83 bp, synthetic construct
  source 1..83: mol_type = other DNA
taagatgtattgaaatatgaccacaaagacacgctcggattagcagtatacgtttaataacaacataagatgtattgaaatta

SEQ ID NO: 128 - DNA, 65 bp, synthetic construct
  source 1..65: mol_type = other DNA
attgaaatatgaccacaaagacacgctcggattagcagtatacgtttaataacaacataagatgt

SEQ ID NO: 129 - RNA, 65 nt, synthetic construct
  source 1..65: mol_type = other RNA
attgaaatatgaccacaaagacacgctcggattagcagtatacgtttaataacaacataagatgt

SEQ ID NO: 130 - DNA, 123 bp, synthetic construct
  source 1..123: mol_type = other DNA
gctgaagggcatcgacttcaaggaggacggcaacatcctggggcacaagctggagtacaactacaacagccacaacgtctatatcatggccgacaagcagaagaacggcatcaaggtgaactt

SEQ ID NO: 131 - DNA, 96 bp, synthetic construct
  source 1..96: mol_type = other DNA
gtttaataacaacataagatgtattgaaatacttcaaggaggacggcaacatcctggggcacaagcgtttaataacaacataagatgtattgaaat

SEQ ID NO: 132 - DNA, 96 bp, synthetic construct
  source 1..96: mol_type = other DNA
gtttaataacaacataagatgtattgaaataggccgacaagcagaagaacggcatcaaggtgaactgtttaataacaacataagatgtattgaaat

(19) United States
(12) Patent Application Publication
(10) Pub. No.: US 2024/0376461 A1
(43) Pub. Date: Nov. 14, 2024

(54) NOVEL TYPE I-B CRISPR-CAS SYSTEM FROM CLOSTRIDIA

(71) Applicant: NORTH CAROLINA STATE UNIVERSITY, Raleigh, NC (US)

(72) Inventors: Rodolphe BARRANGOU, Raleigh, NC (US); Claudio Hidalgo CANTABRANA, Cary, NC (US); Matthew A. NETHERY, Raleigh, NC (US)

(21) Appl. No.: 18/691,108

(22) PCT Filed: Sep. 12, 2022
(86) PCT No.: PCT/US2022/076260

§ 371 Date: Mar. 12, 2024

Related U.S. Application Data
(60) Provisional application No. 63/243,505, filed on Sep. 13, 2021.

(51) Int. Cl. C12N 15/10; C12N 9/22; C12N 15/11

(52) U.S. Cl. CPC C12N 15/102; C12N 9/22; C12N 15/1079; C12N 15/11; C12N 2310/20

(57) ABSTRACT

This invention is directed to recombinant Clustered Regularly Interspaced Short Palindromic Repeats (CRISPR) and recombinant nucleic acid constructs encoding clostridia Type I-B CASCADE complexes, expression cassettes and vectors comprising the same, and methods of use thereof for modifying genomes, altering expression, killing one or more cells in a population of cells, and screening or selecting for genomic variants of an organism.

### STATEMENT OF PRIORITY

This application claims the benefit, under 35 U.S.C. § 119 (e), of U.S. Provisional Application No. 63/243,505 filed on Sep. 13, 2021, the entire contents of which is incorporated by reference herein.

### STATEMENT REGARDING ELECTRONIC FILING OF A SEQUENCE LISTING

A Sequence Listing in XML text format, entitled 5051-991WO.xml, 139,732 bytes in size, generated on Sep. 9, 2022 and filed herewith, is hereby incorporated by reference into the specification for its disclosures.

### FIELD OF THE INVENTION

This invention relates to recombinant Clustered Regularly Interspaced Short Palindromic Repeats (CRISPR) and recombinant nucleic acid constructs encoding clostridia Type I-B CASCADE complexes, expression cassettes and vectors comprising the same, and methods of use thereof for modifying genomes, altering gene expression, killing one or more cells in a population of cells, and screening or selecting for genomic variants of an organism.

### BACKGROUND OF THE INVENTION

Clustered Regularly Interspaced Short Palindromic Repeats (CRISPR), in combination with CRISPR-associated genes (cas) constitute the CRISPR-Cas system, which confers adaptive immunity in many bacteria and most archaea. CRISPR-mediated immunization occurs through the integration of DNA from invasive genetic elements such as plasmids and phages that can be used to thwart future infections by invaders containing the same sequence.

CRISPR-Cas systems consist of CRISPR arrays of short DNA “repeats” interspaced by hypervariable “spacer” sequences and a set of flanking cas genes. The system acts by providing adaptive immunity against invasive genetic elements such as phage and plasmids through the sequence-specific targeting and interference of foreign nucleic acids (Barrangou et al. 2007. Science. 315:1709-1712; Brouns et al. 2008. Science 321:960-4; Horvath and Barrangou. 2010. Science. 327:167-70; Marraffini and Sontheimer. 2008. Science. 322:1843-1845; Bhaya et al. 2011. Annu. Rev. Genet. 45:273-297; Terns and Terns. 2011. Curr. Opin. Microbiol. 14:321-327; Westra et al. 2012. Annu. Rev. Genet. 46:311-339; Barrangou R. 2013. RNA. 4:267-278). Typically, invasive DNA sequences are acquired as novel “spacers” (Barrangou et al. 2007. Science. 315:1709-1712), each paired with a CRISPR repeat and inserted as a novel repeat-spacer unit in the CRISPR locus. The “spacers” are acquired by the Cas1 and Cas2 proteins that are universal to all CRISPR-Cas systems (Makarova et al. 2011. Nature Rev. Microbiol. 9:467-477; Yosef et al. 2012. Nucleic Acids Res. 40:5569-5576), with involvement by the Cas4 protein in some systems (Plagens et al. 2012. J. Bact. 194:2491-2500; Zhang et al. 2012. PLoS One 7: e47232). The resulting repeat-spacer array is transcribed as a long pre-CRISPR RNA (pre-CRISPR, pre-crRNA) (Brouns et al. 2008. Science 321:960-4), which is processed into CRISPR RNAs (CRISPRs, crRNAs) that drive sequence-specific recognition of DNA or RNA. Specifically, crRNAs guide nucleases towards complementary targets for sequence-specific nucleic acid cleavage mediated by Cas endonucleases (Garneau et al. 2010. Nature. 468:67-71; Haurwitz et al. 2010. Science. 329:1355-1358; Sapranauskas et al. 2011. Nucleic Acid Res. 39:9275-9282; Jinek et al. 2012. Science. 337:816-821; Gasiunas et al. 2012. Proc. Natl. Acad. Sci. 109: E2579-E2586; Magadan et al. 2012. PLoS One. 7: e40913; Karvelis et al. 2013. RNA Biol. 10:841-851).

These widespread systems occur in nearly half of bacteria (about 46%) and the large majority of archaea (about 90%). CRISPR/Cas are subdivided in classes and types based on the cas gene content, organization and variation in the biochemical processes that drive crRNA biogenesis, and Cas protein complexes that mediate target recognition and cleavage. Class 1 uses multiple Cas proteins in a cascade complex to degrade nucleic acids (see, FIG. 1). Class 2 uses a single large Cas protein to degrade nucleic acids. The type I systems are the most prevalent in bacteria and in archaea (Makarova et al. 2011. Nature Rev. Microbiol. 9:467-477) and target DNA (Brouns et al. 2008. Science 321:960-4). A complex of 3-8 Cas proteins called the CRISPR associated complex for antiviral defense (Cascade) processes the pre-crRNAs (Brouns et al. 2008. Science 321:960-4), retaining the crRNA to recognize DNA sequences called “protospacers” that are complementary to the spacer portion of the crRNA. Aside from complementarity between the crRNA spacer and the protospacer, targeting requires a protospacer-adjacent motif (PAM) located at the 5′ end of the protospacer (Mojica et al. 2009. Microbiology 155:733-740; Sorek et al. 2013. Ann. Rev. Biochem. 82:237-266). For type I systems, the PAM is directly recognized by Cascade (Sashital et al. 2012. Mol. Cell 46:606-615; Westra et al. 2012. Mol. Cell 46:595-605). The exact PAM sequence that is required can vary between different type I systems. Once a protospacer is recognized, Cascade generally recruits the endonuclease Cas3, which cleaves and degrades the target DNA (Sinkunas et al. 2011. EMBO J. 30:1335-1342; Sinkunas et al. 2013. EMBO J. 32:385-394).

### SUMMARY OF THE INVENTION

A first aspect of the invention provides a recombinant nucleic acid construct comprising a Clustered Regularly Interspaced Short Palindromic Repeats (CRISPR) RNA comprising one or more (e.g., 1, 2, 3, 4, 5, 6, 7, 8, 9, 10 or more) repeat sequence(s) and one or more (e.g., 1, 2, 3, 4, 5, 6, 7, 8, 9, 10 or more) spacer sequence(s), wherein each spacer sequence is linked at least at its 5′ end to a repeat sequence (e.g., a spacer-repeat, or repeat-spacer-repeat, and the like), and the spacer sequence is complementary to a target sequence (protospacer) in a nucleic acid (e.g., DNA) of an organism, wherein the target sequence is located immediately adjacent (3′) to a protospacer adjacent motif (PAM). The invention further provides expression cassettes and vectors comprising the recombinant nucleic acid constructs of the invention.

A second aspect of the invention provides a protein-RNA complex comprising: (a) a Clustered Regularly Interspaced Short Palindromic Repeats (CRISPR) comprising one or more repeat sequences and one or more spacer sequence(s), wherein each spacer sequence is linked at least at its 5′ end to a repeat sequence (e.g., a spacer-repeat), and the spacer sequence is complementary to a target sequence (protospacer) in a target nucleic acid of a target organism, wherein the target sequence is located immediately adjacent (3′) to a protospacer adjacent motif (PAM); and (b) a Type I-B CRISPR associated complex for antiviral defense complex (Cascade complex) comprising a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1 or SEQ ID NO:19, a Cas8 polypeptide having at least 80% sequence identity to the amino acid sequence of SEQ ID NO: 2 or SEQ ID NO:20, a Cas7 polypeptide having at least 80% sequence identity to the amino acid sequence of SEQ ID NO:3 or SEQ ID NO:21, and a Cas5 polypeptide having at least 80% sequence identity to the amino acid sequence of SEQ ID NO:4 or SEQ ID NO:22, and optionally comprising a Cas3 polypeptide having at least 80% sequence identity to the amino acid sequence of SEQ ID NO:5 or SEQ ID NO:23.

In a third aspect, a method of modifying (editing) the genome of a target organism is provided, the method comprising introducing into the target organism or a cell of the target organism (a) a recombinant nucleic acid construct comprising a Clustered Regularly Interspaced Short Palindromic Repeats (CRISPR) comprising one or more repeat sequences and one or more spacer sequence(s), wherein each spacer sequence is linked at least at its 5′ end to a repeat sequence, and the spacer sequence is complementary to a target sequence (protospacer) in a target nucleic acid of a target organism, wherein the target sequence is located immediately adjacent (3′) to a protospacer adjacent motif (PAM); (b) a recombinant nucleic acid construct encoding: a Type I-B CRISPR associated complex for antiviral defense complex (Cascade complex) comprising: (i) a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO: 1 or SEQ ID NO:19, a Cas8 polypeptide having at least 80% sequence identity to the amino acid sequence of SEQ ID NO:2 or SEQ ID NO:20, a Cas7 polypeptide having at least 80% sequence identity to the amino acid sequence of SEQ ID NO: 3 or SEQ ID NO:21, and a Cas5 polypeptide having at least 80% sequence identity to the amino acid sequence of SEQ ID NO:4 or SEQ ID NO:22, and a Cas3 polypeptide having at least 80% sequence identity to the amino acid sequence of SEQ ID NO:5 or SEQ ID NO: 23; and (c) a repair template, thereby modifying the genome of the target organism.

A fourth aspect of the invention provides a method of modifying the genome of a bacterial cell that comprises an endogenous Type I-B CRISPR-Cas system, the method comprising introducing into the bacterial cell (a) a recombinant nucleic acid construct comprising a Clustered Regularly Interspaced Short Palindromic Repeats (CRISPR) comprising one or more repeat sequences and one or more spacer sequence(s), wherein each spacer sequence is linked at least at its 5′ end to a repeat sequence, and the spacer sequence is complementary to a target sequence (protospacer) in a nucleic acid of a target organism, wherein the target sequence is located immediately adjacent (3′) to a protospacer adjacent motif (PAM); and (b) a repair template, thereby modifying the genome of the bacterial cell.

In a fifth aspect of the invention, a method of modifying (editing) the genome of a target organism is provided, the method comprising introducing into the target organism or a cell of the target organism a protein-RNA complex, the protein-RNA complex comprising: (a) a Clustered Regularly Interspaced Short Palindromic Repeats (CRISPR) comprising one or more repeat sequences and one or more spacer sequence(s), wherein each spacer sequence is linked at least at its 5′ end to a repeat sequence, and the spacer sequence is complementary to a target sequence (protospacer) in a target nucleic acid of a target organism, wherein the target sequence is located immediately adjacent (3′) to a protospacer adjacent motif (PAM); (b) a Type I-B CRISPR associated complex for antiviral defense complex (Cascade complex) comprising (i) a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1 or SEQ ID NO:19, a Cas8 polypeptide having at least 80% sequence identity to the amino acid sequence of SEQ ID NO:2 or SEQ ID NO:20, a Cas7 polypeptide having at least 80% sequence identity to the amino acid sequence of SEQ ID NO:3 or SEQ ID NO: 21, and a Cas5 polypeptide having at least 80% sequence identity to the amino acid sequence of SEQ ID NO:4 or SEQ ID NO:22, and a Cas3 polypeptide having at least 80% sequence identity to the amino acid sequence of SEQ ID NO:5 or SEQ ID NO:23; and (c) a repair template, thereby modifying the genome of the target organism.

In a sixth aspect of the invention, a method of altering the expression (repressing expression/overexpression) of a target gene in a target organism is provided, the method comprising introducing into the target organism or a cell of the target organism (a) a recombinant nucleic acid construct comprising a Clustered Regularly Interspaced Short Palindromic Repeats (CRISPR) comprising one or more repeat sequences and one or more spacer sequence(s), wherein each spacer sequence is linked at least at its 5′ end to a repeat sequence, and the spacer sequence is complementary to a target sequence (protospacer) in a nucleic acid of a target organism, wherein the target sequence is located immediately adjacent (3′) to a protospacer adjacent motif (PAM); (b) a recombinant nucleic acid construct encoding a Type I-B CRISPR associated complex for antiviral defense complex (Cascade complex) comprising: a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO: 1 or SEQ ID NO: 19, a Cas8 polypeptide having at least 80% sequence identity to the amino acid sequence of SEQ ID NO:2 or SEQ ID NO:20, a Cas7 polypeptide having at least 80% sequence identity to the amino acid sequence of SEQ ID NO:3 or SEQ ID NO: 21, and a Cas5 polypeptide having at least 80% sequence identity to the amino acid sequence of SEQ ID NO:4 or SEQ ID NO:2, thereby altering expression of the target gene in the cell of the target organism.

A seventh aspect of the invention provides a method of altering the expression (repressing expression/overexpression) of a target gene in a target organism, the method comprising introducing into the target organism or a cell of the target organism a protein-RNA complex, the protein-RNA complex comprising: (a) a Clustered Regularly Interspaced Short Palindromic Repeats (CRISPR) comprising one or more repeat sequences and one or more spacer sequence(s), wherein each spacer sequence is linked at least at its 5′ end to a repeat sequence, and the spacer sequence is complementary to a target sequence (protospacer) in a target nucleic acid of a target organism, wherein the target sequence is located immediately adjacent (3′) to a protospacer adjacent motif (PAM); and (b) a Type I-B CRISPR associated complex for antiviral defense complex (Cascade complex) comprising a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1 or SEQ ID NO:19, a Cas8 polypeptide having at least 80% sequence identity to the amino acid sequence of SEQ ID NO:2 or SEQ ID NO:20, a Cas7 polypeptide having at least 80% sequence identity to the amino acid sequence of SEQ ID NO:3 or SEQ ID NO:21, and a Cas5 polypeptide having at least 80% sequence identity to the amino acid sequence of SEQ ID NO: 4 or SEQ ID NO:2, thereby altering expression of the target gene in the cell of the target organism.

An eighth aspect of the invention provides a method of screening for a variant cell of an organism, the method comprising (a) introducing into a population of cells from (or of) the organism (i) a recombinant nucleic acid construct comprising a Clustered Regularly Interspaced Short Palindromic Repeats (CRISPR) comprising one or more repeat sequences and one or more spacer sequence(s), wherein each spacer sequence is linked at least at its 5′ end to a repeat sequence, and the spacer sequence is complementary to a target sequence (protospacer) in a target nucleic acid of at least a portion of the population of cells of the organism and the target sequence is not present in the variant cell, wherein the target sequence is located immediately adjacent (3′) to a protospacer adjacent motif (PAM): (ii) a recombinant nucleic acid construct encoding: a Cas3 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:5 or SEQ ID NO:23, and a Type I-B CRISPR associated complex for antiviral defense complex (Cascade complex) comprising (i) a Cas6 polypeptide having at least 80% sequence identity to the amino acid sequence of SEQ ID NO: 1 or SEQ ID NO:19, a Cas8 polypeptide having at least 80% sequence identity to the amino acid sequence of SEQ ID NO:2 or SEQ ID NO:20, a Cas7 polypeptide having at least 80% sequence identity to the amino acid sequence of SEQ ID NO:3 or SEQ ID NO:21, and a Cas5 polypeptide having at least 80% sequence identity to the amino acid sequence of SEQ ID NO: 4 or SEQ ID NO:22; wherein the recombinant nucleic acid construct comprising a CRISPR and the recombinant nucleic acid construct encoding a Cascade complex each comprise a polynucleotide encoding a polypeptide conferring resistance to a selection agent, thereby killing transformed cells comprising the target sequence and producing a subpopulation of cells of the population of cells; and (b) selecting from the subpopulation of cells produced in (a) one or more cells that are resistant to the selection agent, thereby selecting one or more variant cells that do not comprise the target sequence and are not killed.

In a ninth aspect, the present invention provides a method of screening for variant bacterial cells comprising an endogenous Type I-B CRISPR-Cas system, the method comprising (a) introducing into a population of bacterial cells a recombinant nucleic acid construct comprising a Clustered Regularly Interspaced Short Palindromic Repeats (CRISPR) comprising one or more repeat sequences and one or more spacer sequence(s), wherein each spacer sequence is linked at least at its 5′ end to a repeat sequence, and the spacer sequence is complementary to a target sequence (protospacer) in a nucleic acid of the bacteria wherein the target sequence is not present in the variant cell and the target sequence is located immediately adjacent (3′) to a protospacer adjacent motif (PAM); and wherein the recombinant nucleic acid construct comprising a CRISPR comprises a polynucleotide encoding a polypeptide conferring resistance to a selection agent, thereby killing transformed cells comprising the target sequence and producing a subpopulation of bacterial cells; and (b) selecting from the subpopulation of bacterial cells produced in (a) one or more bacterial cells that are resistant to the selection agent, thereby selecting one or more variant bacterial cells that do not comprise the target sequence and are not killed.

A tenth aspect provides a method of screening for a variant cell of an organism, the method comprising (a) introducing into a population of cells from (or of) the organism a protein-RNA complex, the protein-RNA complex comprising: (i) a Clustered Regularly Interspaced Short Palindromic Repeats (CRISPR) comprising one or more repeat sequences and one or more spacer sequence(s), wherein each spacer sequence is linked at least at its 5′ end to a repeat sequence, and the spacer sequence is complementary to a target sequence (protospacer) in a target nucleic acid of at least a portion of the population of cells of the organism wherein the target sequence is not present in the variant cell, wherein the target sequence is located immediately adjacent (3′) to a protospacer adjacent motif (PAM); (ii) a Cas3 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:5 or SEQ ID NO:23, and a Type I-B CRISPR associated complex for antiviral defense complex (Cascade complex) comprising (i) a Cas6 polypeptide having at least 80% sequence identity to the amino acid sequence of SEQ ID NO:1 or SEQ ID NO:19, a Cas8 polypeptide having at least 80% sequence identity to the amino acid sequence of SEQ ID NO:2 or SEQ ID NO: 20, a Cas7 polypeptide having at least 80% sequence identity to the amino acid sequence of SEQ ID NO:3 or SEQ ID NO:21, and a Cas5 polypeptide having at least 80% sequence identity to the amino acid sequence of SEQ ID NO:4 or SEQ ID NO:22, wherein the recombinant nucleic acid construct comprising a CRISPR and the recombinant nucleic acid construct encoding a Cascade complex each comprise a polynucleotide encoding a polypeptide conferring resistance to a selection agent, thereby killing transformed cells comprising the target sequence and producing a subpopulation of cells of the population of cells; and (b) selecting from the subpopulation of cells produced in (a) one or more cells that are resistant to the selection agent, thereby selecting one or more variant cells that do not comprise the target sequence and are not killed.

An eleventh aspect of the invention provides a method of killing one or more cells in a population of bacterial and/or archaeal cells, the method comprising introducing into the one or more cells of the population of bacterial and/or archaeal cells: (a) a recombinant nucleic acid construct comprising a Clustered Regularly Interspaced Short Palindromic Repeats (CRISPR) comprising one or more repeat sequences and one or more spacer nucleotide sequence(s), wherein each of the one or more spacer sequences comprises a 3′ end and a 5′ end and is linked at least at its 5′ end to a repeat sequence, and each of the one or more spacer sequences is complementary to a target sequence (protospacer) in the genome of the bacterial and/or archaeal cells of the population, wherein the target sequence is a genomic sequence that is conserved among the one or more cells within the population of bacterial and/or archaeal cells and the target sequence is located immediately adjacent (3′) to a protospacer adjacent motif (PAM); and (b) a recombinant nucleic acid construct encoding: a Cas3 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:5 or SEQ ID NO:23, and a Type I-B CRISPR associated complex for antiviral defense complex (Cascade complex) comprising (i) a Cas6 polypeptide having at least 80% sequence identity to the amino acid sequence of SEQ ID NO:1 or SEQ ID NO: 19, a Cas8 polypeptide having at least 80% sequence identity to the amino acid sequence of SEQ ID NO:2 or SEQ ID NO: 20, a Cas7 polypeptide having at least 80% sequence identity to the amino acid sequence of SEQ ID NO:3 or SEQ ID NO:21, and a Cas5 polypeptide having at least 80% sequence identity to the amino acid sequence of SEQ ID NO:4 or SEQ ID NO:22, thereby killing one or more cells in the population of bacterial and/or archaeal cells that comprise the target sequence in their genome.

A twelfth aspect provides a method of killing one or more cells in a population of bacterial and/or archaeal cells that comprise an endogenous Type I-B Clustered Regularly Interspaced Short Palindromic Repeats (CRISPR)-Cas system, the method comprising introducing into the one or more cells of the population of bacterial and/or archaeal cells a recombinant nucleic acid construct comprising a CRISPR comprising one or more repeat sequences and one or more spacer nucleotide sequence(s), wherein each of the one or more spacer sequences comprises a 3′ end and a 5′ end and is linked at least at its 5′ end to a repeat sequence, and each of the one or more spacer sequences is complementary to a target sequence (protospacer) in a target DNA in the one or more bacterial and/or archaeal cells of the population, wherein the target sequence is conserved among the one or more cells within the population of bacterial and/or archaeal cells and the target sequence is located immediately adjacent (3′) to a protospacer adjacent motif (PAM), thereby killing the one or more cells within the population of bacterial and/or archaeal cells that comprise the target sequence in their genome.

A thirteenth aspect provides a method of killing one or more cells in a population of bacterial and/or archaeal cells, the method comprising introducing into the one or more cells of the population of bacterial and/or archaeal cells a protein-RNA complex, the protein-RNA complex comprising: (a) a Clustered Regularly Interspaced Short Palindromic Repeats (CRISPR) comprising one or more repeat sequences and one or more spacer nucleotide sequence(s), wherein each of the one or more spacer sequences comprises a 3′ end and a 5′ end and is linked at least at its 5′ end to a repeat sequence, and each of the one or more spacer sequences is complementary to a target sequence (protospacer) in the genome of the bacterial and/or archaeal cells of the population, wherein the target sequence is a genomic sequence that is conserved among the one or more cells within the population of bacterial and/or archaeal cells and the target sequence is located immediately adjacent (3′) to a protospacer adjacent motif (PAM); and (b) a Type I-B CRISPR associated complex for antiviral defense complex (Cascade complex) comprising (i) a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1 or SEQ ID NO:19, a Cas8 polypeptide having at least 80% sequence identity to the amino acid sequence of SEQ ID NO:2 or SEQ ID NO:20, a Cas7 polypeptide having at least 80% sequence identity to the amino acid sequence of SEQ ID NO: 3 or SEQ ID NO:21, and a Cas5 polypeptide having at least 80% sequence identity to the amino acid sequence of SEQ ID NO:4 or SEQ ID NO:22, and a Cas3 polypeptide having at least 80% sequence identity to the amino acid sequence of SEQ ID NO:5 or SEQ ID NO: 23, thereby killing one or more cells in the population of bacterial and/or archaeal cells that comprise the target sequence in their genome.

Further provided are cells and/or organisms produced (e.g., modified) by the methods of the invention and nucleic acid constructs for carrying out the methods. These and other aspects of the invention are set forth in more detail in the description of the invention below.

### BRIEF DESCRIPTION OF THE DRAWINGS

FIGS. 1A-1B provides a schematic representation of CRISPR-Cas loci architecture of Clostridium spp. 1141A1FAA Type I-B (FIG. 1A) and Erysipelatoclostridium ramosum Type I-B (FIG. 1B).

FIG. 2 provides a 16S phylogenetic tree of several clostridium species and E. coli.

FIG. 3 shows PAM prediction data for Clostridium spp. 1141A1FAA.

FIG. 4 shows PAM prediction data for Erysipelatoclostridium ramosum.

FIG. 5 provides E. ramosum DSM 1402 type I-B mRNA-seq reads data.

FIG. 6 provides E. ramosum DSM 1402 type I-B smRNA-seq reads showing CRISPR array transcription and mature crRNA biogenesis.

FIG. 7 shows boundaries of mature crRNAs of E. ramosum DSM 1402 and the sequence of the mature crRNA of E. ramosum DSM 1402 at the bottom of the figure along with the hairpin structure of the mature crRNA.

FIG. 8 shows targeting using a CRISPR array having a spacer that is complementary to the target sequence having the PAM sequence of ATCG flanking the 5′ edge of the protospacer (upper panel) and a CRISPR array having a spacer that is complementary to the target sequence having the PAM sequence of ATCA flanking the 5′ edge of the protospacer (lower panel).

FIG. 9 shows round 1 testing 0.5 nM E. ramosum Type 1-B PAM ATCA or ATCG plasmid using a non-targeting plasmid as a negative control.

FIG. 10 shows round 2 testing 0.5 nM E. ramosum Type 1-B PAM ATCA or ATCG plasmid using a non-targeting plasmid as a negative control.

FIG. 11 shows round 2 testing 0.5 nM E. ramosum Type 1-B PAM ATCA or ATCG plasmid using a non-targeting plasmid as a negative control.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancement,” “improve” and “improvement” (and the like and grammatical variations thereof) describe an elevation of at least about 5%, 10%, 15%, 20%, 25%, 50%, 75%, 100%, 150%, 200%, 300%, 400%, 500%, 750%, 1000%, 2500%, 5000%, 10,000%, 20,000% or more as compared to a control (e.g., a CRISPR targeting a particular gene having, for example, more spacer sequences targeting different regions of that gene and therefore having increased repression of that gene as compared to a CRISPR targeting the same gene but having, for example, fewer spacer sequences targeting different regions of that gene).

As used herein, the terms “reduce,” “reduced,” “reducing,” “reduction,” “diminish,” “suppress,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount. As an example, a mutation in a Cas3 nuclease can reduce the nuclease activity of the Cas3 by at least about 90%, 95%, 97%, 98%, 99%, or 100% as compared to a control (e.g., wild-type Cas3).

The terms “complementary” or “complementarity,” as used herein, refer to the natural binding of polynucleotides under permissive salt and temperature conditions by base-pairing. For example, the sequence “A-G-T” binds to the complementary sequence “T-C-A.” Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement” as used herein can mean 100% complementarity with the comparator nucleotide sequence or it can mean less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

As used herein, the phrase “substantially complementary,” or “substantial complementarity” in the context of two nucleic acid molecules, nucleotide sequences or protein sequences, refers to two or more sequences or subsequences that are at least about 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue complementary, when compared and aligned for maximum correspondence, as measured using one of the following sequence comparison algorithms or by visual inspection. In some embodiments, substantial complementarity can refer to two or more sequences or subsequences that have at least about 80%, at least about 85%, at least about 90%, at least about 95, 96, 96, 97, 98, or 99% complementarity (e.g., about 80% to about 90%, about 80% to about 95%, about 80% to about 96%, about 80% to about 97%, about 80% to about 98%, about 80% to about 99% or more, about 85% to about 90%, about 85% to about 95%, about 85% to about 96%, about 85% to about 97%, about 85% to about 98%, about 85% to about 99% or more, about 90% to about 95%, about 90% to about 96%, about 90% to about 97%, about 90% to about 98%, about 90% to about 99% or more, about 95% to about 97%, about 95% to about 98%, about 95% to about 99% or more). Two nucleotide sequences can be considered to be substantially complementary when the two sequences hybridize to each other under stringent conditions. In some representative embodiments, two nucleotide sequences considered to be substantially complementary hybridize to each other under highly stringent conditions.

As used herein, “contact,” contacting,” “contacted,” and grammatical variations thereof, refers to placing the components of a desired reaction together under conditions suitable for carrying out the desired reaction (e.g., integration, transformation, site-specific cleavage (nicking, cleaving), amplifying, site specific targeting of a polypeptide of interest and the like). The methods and conditions for carrying out such reactions are well known in the art (See, e.g., Gasiunas et al. (2012) Proc. Natl. Acad. Sci. 109: E2579-E2586; M. R. Green and J. Sambrook (2012) Molecular Cloning: A Laboratory Manual. 4th Ed., Cold Spring Harbor Laboratory Press, Cold Spring Harbor, NY).

As used herein, the term “commensal bacteria” refers to a bacterium that is naturally present in a microbiome, such as in the gut microbiome of a host (e.g., human gut microbiome), without causing harm to the host. In some cases, a commensal bacterium may confer a benefit to the host organism.

As used herein, Type I Clustered Regularly Interspaced Short Palindromic Repeats (CRISPR)-associated complex for antiviral defense (Cascade) refers to a complex of polypeptides involved in processing of pre-crRNAs and subsequent binding to the target DNA in type I CRISPR-Cas systems. Exemplary Type I-B polypeptides useful with this invention include a Cas6 polypeptide (SEQ ID NO:1 or SEQ ID NO:19), a Cas8 polypeptide (SEQ ID NO:2 or SEQ ID NO:20), a Cas7 polypeptide (SEQ ID NO:3 or SEQ ID NO:21), a Cas5 polypeptide (SEQ ID NO:4 or SEQ ID NO:22), a Cas3 polypeptide (SEQ ID NO:5 or SEQ ID NO:23). Type I-B Cascade polypeptides that function in spacer acquisition include Cas1 (SEQ ID NO:7 or SEQ ID NO:15), Cas2 (SEQ ID NO: 8 or SEQ ID NO: 15) and Cas4 (SEQ ID NO:6 or SEQ ID NO:14). In some embodiments of this invention, a recombinant nucleic acid construct may comprise, consist essentially of, or consist of a recombinant nucleic acid encoding a subset of Type I-B Cascade polypeptides that function to process a CRISPR and subsequently bind to a target DNA using the spacer of the processed CRISPR as a guide. A further Type I-B polypeptide useful with this invention includes a Cas3 nuclease.

In some embodiments of this invention, a recombinant nucleic acid construct may comprise, consist essentially of, or consist of a recombinant nucleic acid encoding a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1 or SEQ ID NO: 19, a Cas8 polypeptide having at least 80% sequence identity to the amino acid sequence of SEQ ID NO:2 or SEQ ID NO:20, a Cas7 polypeptide having at least 80% sequence identity to the amino acid sequence of SEQ ID NO:3 or SEQ ID NO:21, and a Cas5 polypeptide having at least 80% sequence identity to the amino acid sequence of SEQ ID NO: 4 or SEQ ID NO:22 and optionally encoding a Cas3 polypeptide having at least 80% sequence identity to the amino acid sequence of SEQ ID NO:5 or SEQ ID NO:23.

A “fragment” or “portion” of a nucleic acid will be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising a nucleotide sequence of contiguous nucleotides that are identical or almost identical (e.g., about 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the invention may be, where appropriate, included in a larger polynucleotide of which it is a constituent. In some embodiments, a fragment of a polynucleotide can be a fragment that encodes a polypeptide that retains its function (e.g., encodes a fragment of a Type I-B Cascade polypeptide that is reduce in length as compared to the wild type polypeptide but which retains at least one function of a Type I-B Cascade protein; e.g., processes CRISPR nucleic acids, bind DNA and/or form a complex). In some embodiments, a fragment of a polynucleotide can be a fragment of a native repeat sequence (e.g., a native repeat sequence from for example, Erysipelatoclostridium ramosum or Clostridium spp. 1141A1FAA)) that is shortened by about 1 nucleotide to about 7 nucleotides (e.g., 1, 2, 3, 4, 5, 6, or 7) or by about 1 nucleotide to about 8 nucleotides (e.g., 1, 2, 3, 4, 5, 6, 7 or 8) from the 3′ end of a native repeat sequence). In some embodiments, a fragment of a polynucleotide can be a fragment of a native repeat sequence that remains at the 3′ end of a spacer (e.g., from the 5′ end of the native repeat) when the native repeat sequence is shortened by 1 nucleotide to about 7 nucleotides or by 1 nucleotide to about 8 nucleotides from the 3′ end of a native repeat sequence (e.g., a portion of a repeat sequence having a length of about 11, 12, 13, 14, 15, 16, 17, 18, 19, 20 21, 22, 23, 24, 25, or 26 nucleotides).

As used herein, “chimeric” refers to a nucleic acid molecule or a polypeptide in which at least two components are derived from different sources (e.g., different organisms, different coding regions).

A “heterologous” or a “recombinant” nucleic acid is an exogenous nucleic acid not naturally associated with a host cell into which it is introduced, including non-naturally occurring multiple copies of a naturally occurring nucleic acid. In some embodiments, “heterologous” may include a nucleic acid that is endogenous to a host cell but is in a non-natural position relative to the wild type as a result of human intervention.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to said nucleotide sequence of the invention.

As used herein, hybridization, hybridize, hybridizing, and grammatical variations thereof, refer to the binding of two complementary nucleotide sequences or substantially complementary sequences in which some mismatched base pairs are present. The conditions for hybridization are well known in the art and vary based on the length of the nucleotide sequences and the degree of complementarity between the nucleotide sequences. In some embodiments, the conditions of hybridization can be high stringency, or they can be medium stringency or low stringency depending on the amount of complementarity and the length of the sequences to be hybridized. The conditions that constitute low, medium and high stringency for purposes of hybridization between nucleotide sequences are well known in the art (See, e.g., Gasiunas et al. (2012) Proc. Natl. Acad. Sci. 109: E2579-E2586; M. R. Green and J. Sambrook (2012) Molecular Cloning: A Laboratory Manual. 4th Ed., Cold Spring Harbor Laboratory Press, Cold Spring Harbor, NY).

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is a mRNA that is naturally occurring in or endogenous to the organism. A “homologous” nucleic acid is a nucleic acid naturally associated with a host cell into which it is introduced.

As used herein, the terms “nucleic acid,” “nucleic acid molecule,” “nucleic acid construct,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The nucleic acid constructs of the present disclosure can be DNA or RNA. In some embodiments, the nucleic acid constructs of the present disclosure are DNA. Thus, although the nucleic acid constructs of this invention may be described and used in the form of DNA, depending on the intended use, they may also be described and used in the form of RNA.

As used herein, the term “gene” refers to a nucleic acid molecule capable of being used to produce mRNA, tRNA, tRNA, miRNA, anti-microRNA, regulatory RNA, and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

A “synthetic” nucleic acid or nucleotide sequence, as used herein, refers to a nucleic acid or nucleotide sequence that is not found in nature but is constructed by human intervention, and as a consequence, it is not a product of nature.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide,” and “polynucleotide” are also used interchangeably herein to refer to a heteropolymer of nucleotides. Except as otherwise indicated, nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end. Thus, for example, an element in the 3′ region of a polynucleotide can be located anywhere from the first nucleotide located at the 3′ end of the polynucleotide to the nucleotide located halfway through the polynucleotide. An element that is described as being “at the 5′ end” or “at the 3′ end” of a polynucleotide (5′ to 3′) refers to an element located immediately adjacent to (upstream of) the first nucleotide at the 5′ end of the polynucleotide, or immediately adjacent to (downstream of) the last nucleotide located at the 3′ end of the polynucleotide, respectively.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used herein, a “hairpin sequence” is a nucleotide sequence comprising hairpins. A hairpin (e.g., stem-loop, fold-back) refers to a nucleic acid molecule having a secondary structure that includes a region of nucleotides that form a single strand that are further flanked on either side by a double stranded-region. Such structures are well known in the art. As known in the art, the double stranded region can comprise some mismatches in base pairing or can be perfectly complementary. In some embodiments, a repeat sequence may comprise, consist essentially of, consist of a hairpin sequence that is located within the repeat nucleotide sequence (i.e., at least one nucleotide (e.g., one, two, three, four, five, six, seven, eight, nine, ten, or more) of the repeat nucleotide sequence is present on either side of the hairpin that is within the repeat nucleotide sequence).

A “CRISPR” as used herein comprises one or more repeat sequences and one or more spacer sequence(s), wherein each of the one or more spacer sequences is linked at least at its 5′-end to a repeat sequence or portion thereof. A “CRISPR” can include a CRISPR, an unprocessed CRISPR, or a mature/processed CRISPR or a CRISPR that comprises one repeat, or a portion thereof, and a spacer (e.g., repeat-spacer). A “CRISPR” as used herein refers to a nucleic acid molecule that comprises at least one CRISPR repeat sequence, or a portion(s) thereof, and at least one spacer sequence, wherein one of the two repeat sequences, or a portion thereof, is linked to the 5′ end of the spacer sequence and the other of the two repeat sequences, or portion thereof, is linked to the 3′ end of the spacer sequence. In some embodiments, in a recombinant CRISPR of the invention, the combination of repeat nucleotide sequences and spacer sequences is synthetic and not found in nature. A CRISPR may be introduced into a cell or cell free system as RNA, or as DNA in an expression cassette or vector (e.g., plasmid, retrovirus, bacteriophage).

As used herein, the term “spacer sequence” refers to a nucleotide sequence that is complementary to a targeted portion (i.e., “protospacer”) of a nucleic acid or a genome. The term “genome,” as used herein, refers to both chromosomal and non-chromosomal elements (i.e., extrachromosomal (e.g., mitochondrial, plasmid, a chloroplast, and/or extrachromosomal circular DNA (eccDNA))) of a target organism. The spacer sequence guides the CRISPR machinery to the targeted portion of the genome, wherein the targeted portion of the genome may be, for example, modified (e.g., a deletion, an insertion, a single base pair addition, a single base pair substitution, a single base pair removal, a stop codon insertion, and/or a conversion of one base pair to another base pair (base editing)). As another example, the spacer sequence may be used to guide the CRISPR machinery to the targeted portion of the genome, wherein the targeted portion of the genome may be cut and degraded, thereby killing the cell(s) comprising the target sequence.

A “target sequence” or “protospacer” refers to a targeted portion of a genome or of a cell free nucleic acid that is complementary to the spacer sequence of a recombinant CRISPR. A target sequence or protospacer useful with this invention is located immediately adjacent to the 3′ end of a PAM (protospacer adjacent motif) (e.g., 5′-PAM-Protospacer-3′). In some embodiments, a PAM may comprise, consist essentially of, or consist of a sequence of 5′-TTTA-3′, 5′-ATC-3′ or 5′-ATCN-3′ (N=A, T, C, or G, e.g., 5′-ATCG-3′, 5′-ATCA-3′) (e.g., 5′-TTTA-3′ as an example PAM for Clostridium and 5′-ATC-3′ and 5′-ATCN-3′ (5′-ATCG-3′, 5′-ATCA-3′) as example PAMs for E. ramosum). A non-limiting example of a protospacers may be the following, 5′-3′,

(SEQ ID NO: 62)
...ATGCTAATGGAGTTTACTACAAGTTAATCCGGCAAAGCTAAATGGC
CGGCCCGT.

As used herein, the terms “target genome” or “targeted genome” refer to a genome of an organism of interest.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the phrase “substantially identical,” or “substantial identity” in the context of two nucleic acid molecules, nucleotide sequences or protein sequences, refers to two or more sequences or subsequences that have at least about 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity can refer to two or more sequences or subsequences that have at least about 80%, at least about 85%, at least about 90%, at least about 95, 96, 96, 97, 98, or 99% sequence identity.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The following are examples of sets of hybridization/wash conditions that may be used to clone homologous nucleotide sequences that are substantially identical to reference nucleotide sequences of the invention. In one embodiment, a reference nucleotide sequence hybridizes to the “test” nucleotide sequence in 7% sodium dodecyl sulfate (SDS), 0.5 M NaPO₄, 1 mM EDTA at 50° C. with washing in 2×SSC, 0.1% SDS at 50° C. In another embodiment, the reference nucleotide sequence hybridizes to the “test” nucleotide sequence in 7% sodium dodecyl sulfate (SDS), 0.5 M NaPO₄, 1 mM EDTA at 50° C. with washing in 1×SSC, 0.1% SDS at 50° C. or in 7% sodium dodecyl sulfate (SDS), 0.5 M NaPO₄, 1 mM EDTA at 50° C. with washing in 0.5×SSC, 0.1% SDS at 50° C. In still further embodiments, the reference nucleotide sequence hybridizes to the “test” nucleotide sequence in 7% sodium dodecyl sulfate (SDS), 0.5 M NaPO₄, 1 mM EDTA at 50° C. with washing in 0.1×SSC, 0.1% SDS at 50° C., or in 7% sodium dodecyl sulfate (SDS), 0.5 M NaPO₄, 1 mM EDTA at 50° C. with washing in 0.1×SSC, 0.1% SDS at 65° C.

Any polynucleotide and/or nucleic acid construct useful with this invention may be codon optimized for expression in any species of interest. Codon optimization is well known in the art and involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species specific codon usage table with the codons present in the native polynucleotide sequences. As is understood in the art, codon optimization of a nucleotide sequence results in a nucleotide sequence having less than 100% sequence identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function (and in some embodiments, the same structure) as that encoded by the original nucleotide sequence. Thus, in some embodiments of the invention, polynucleotides and/or nucleic acid constructs useful with the invention may be codon optimized for expression in the particular organism/species of interest.

In some embodiments, the polynucleotides and polypeptides of the invention are “isolated.” An “isolated” polynucleotide sequence or an “isolated” polypeptide is a polynucleotide or polypeptide that, by human intervention, exists apart from its native environment and is therefore not a product of nature. An isolated polynucleotide or polypeptide may exist in a purified form that is at least partially separated from at least some of the other components of the naturally occurring organism or virus, for example, the cell or viral structural components or other polypeptides or nucleic acids commonly found associated with the polynucleotide. In representative embodiments, the isolated polynucleotide and/or the isolated polypeptide may be at least about 1%, 5%, 10%, 20%, 30%, 40%, 50%, 60%, 70%, 80%, 90%, 95%, or more pure.

In other embodiments, an isolated polynucleotide or polypeptide may exist in a non-natural environment such as, for example, a recombinant host cell. Thus, for example, with respect to nucleotide sequences, the term “isolated” means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur (e.g., a different host cell, different regulatory sequences, and/or different position in the genome than as found in nature). Accordingly, the polynucleotides and their encoded polypeptides are “isolated” in that, through human intervention, they exist apart from their native environment and therefore are not products of nature, however, in some embodiments, they can be introduced into and exist in a recombinant host cell.

In some embodiments of the invention, a recombinant nucleic acid of the invention comprising/encoding a CRISPR and/or a Cascade complex and/or a Cas3 polypeptide may be operatively associated with a variety of promoters, terminators and other regulatory elements for expression in various organisms or cells. Thus, in some embodiments, at least one promoter and/or at least one terminator may be operably linked to a recombinant nucleic acid of the invention comprising/encoding a CRISPR and/or a Cascade complex and/or a Cas3 polypeptide. In some embodiments, when comprised in the same nucleic acid construct (e.g., expression cassette), the CRISPR and/or recombinant nucleic acid encoding a Cascade complex and/or a Cas3 may be operably linked to separate (independent) promoters that may be the same promoter or a different promoter. In some embodiments, when comprised in the same nucleic acid construct, a CRISPR and a recombinant nucleic acid encoding a Cascade complex and/or a Cas3 polypeptide may be operably linked to a single promoter.

Any promoter useful with this invention can be used and includes, for example, promoters functional with the organism of interest. A promoter useful with this invention can include, but is not limited to, constitutive, inducible, developmentally regulated, tissue-specific/preferred-promoters, and the like, as described herein. A regulatory element as used herein can be endogenous or heterologous. In some embodiments, an endogenous regulatory element derived from the subject organism can be inserted into a genetic context in which it does not naturally occur (e.g., a different position in the genome than as found in nature), thereby producing a recombinant or non-native nucleic acid.

By “operably linked” or “operably associated” as used herein, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

Any promoter that initiates transcription of a recombinant nucleic acid construct of the invention, for example, in an organism/cell of interest may be used. A promoter useful with this invention can include, but is not limited to, a constitutive, inducible, developmentally regulated, tissue-specific/preferred-promoter, and the like, as described herein. A regulatory element as used herein can be endogenous or heterologous. In some embodiments, an endogenous regulatory element derived from the subject organism can be inserted into a genetic context in which it does not naturally occur (e.g., a different position in the genome than as found in nature (e.g., a different position in a chromosome or in a plasmid), thereby producing a recombinant or non-native nucleic acid.

Promoters can include, for example, constitutive, inducible, temporally regulated, developmentally regulated, chemically regulated, tissue-preferred and/or tissue-specific promoters for use in the preparation of recombinant nucleic acid molecules, i.e., “chimeric genes” or “chimeric polynucleotides.” These various types of promoters are known in the art. Thus, expression can be made constitutive, inducible, temporally regulated, developmentally regulated, chemically regulated, tissue-preferred and/or tissue-specific promoters using the recombinant nucleic acid constructs of the invention operatively linked to the appropriate promoter functional in an organism of interest. Expression may also be made reversible using the recombinant nucleic acid constructs of the invention operatively linked to, for example, an inducible promoter functional in an organism of interest. In some embodiments, promoters useful with the constructs of the invention may be any combination of heterologous/exogenous and/or endogenous promoters.

The choice of promoter will vary depending on the quantitative, temporal and spatial requirements for expression, and also depending on the host cell of interest. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

Exemplary promoters include, but are not limited to, promoters functional in eukaryotes and prokaryotes including but not limited to, plants, viruses, bacteria, fungi, archaea, animals, and mammals. For example, promoters useful with archaea include, but are not limited to, Haloferax volcanii tRNA (Lys) promoter (Palmer et al. J. Bacteriol. 1995. 177 (7): 1844-1849), Pyrococcus furiosus gdh promoter (Waege et al. 2010. Appl. Environ. Microbiol. 76:3308-3313), Sulfolobus solfataricus 16S/23S rRNA gene core promoter (DeYoung et al. 2011. FEMS Microbiol. Lett. 321:92-99).

Exemplary promoters useful with yeast can include a promoter from phosphoglycerate kinase (PGK), glyceraldehyde-3-phosphate dehydrogenase (GAP), triose phosphate isomerase (TPI), galactose-regulon (GAL1, GAL10), alcohol dehydrogenase (ADH1, ADH2), phosphatase (PHO5), copper-activated metallothionine (((PI), MFα1, PGK/α2 operator, TPI/α2 operator, GAP/GAL, PGK/GAL, GAP/ADH2, GAP/PHO5, iso-1-cytochrome c/glucocorticoid response element (CYC/GRE), phosphoglycerate kinase/angrogen response element (PGK/ARE), transcription elongation factor EF-1α (TEF1), triose phosphate dehydrogenase (TDH3), phosphoglycerate kinase 1 (PGK1), pyruvate kinase 1 (PYK1), and/or hexose transporter (HXT7) (See, Romanos et al. Yeast 8:423-488 (1992); and Partow et al. Yeast 27:955-964 (2010).

In additional embodiments, a promoter useful with bacteria can include, but is not limited to, L-arabinose inducible (araBAD), P_{BAD}) promoter, any lac promoter, L-rhamnose inducible (rhaP_{BAD}) promoter, T7 RNA polymerase promoter, trc promoter, tac promoter, lambda phage promoter (p_{L}, p_{L}-9G-50), anhydrotetracycline-inducible (tetA) promoter, trp, Ipp, phoA, recA, proU, cst-1, cadA, nar, lpp-lac, cspA, T7-lac operator, T3-lac operator, T4 gene 32, T5-lac operator, nprM-lac operator, Vhb, Protein A, corynebacterial-Escherichia coli like promoters, thr, hom, diphtheria toxin promoter, sig A, sig B, nusG, SoxS, katb, α-amylase (Pamy), Ptms, P43 (comprised of two overlapping RNA polymerase σ factor recognition sites, σA, σB), Ptms, P43, rplK-rplA, ferredoxin promoter, and/or xylose promoter. (See, K. Terpe Appl. Microbiol, Biotechnol. 72:211-222 (2006); Hannig et al. Trends in Biotechnology 16:54-60 (1998); and Srivastava Protein Expr Purif 40:221-229 (2005)).

Translation elongation factor promoters may be used with the invention. Translation elongation factor promoters may include but are not limited to elongation factor Tu promoter (Tuf) (e.g., Ventura et al., Appl. Environ. Microbiol. 69:6908-6922 (2003)), elongation factor P (Pefp) (e.g., Tauer et al., Microbial Cell Factories, 13:150 (2014), rRNA promoters including but not limited to a P3, a P6 a P15 promoter (e.g., Djordjevic et al., Canadian Journal Microbiology, 43:61-69 (1997); Russell and Klaenhammer, Appl. Environ. Microbiol. 67:1253-1261 (2001)) and/or a P11 promoter. In some embodiments, a promoter may be a synthetic promoter derived from a natural promoter (e.g., Rud et al., Microbiology, 152:1011-1019 (2006). In some embodiments, a sakacin promoter may be used with the recombinant nucleic acid constructs of the invention (e.g., Mathiesen et al., J. Appl. Microbial., 96:819-827 (2004).

Non-limiting examples of a promoter functional in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)).

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79:87-94), maize (Christensen et al., 1989. Plant Molec. Biol. 12:619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231:150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP U.S. Pat. No. 255,378). Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), and/or S-adenosyl-L-methionine synthetase (SAMS) (Vander Mijnsbrugge et al. (1996) Plant and Cell Physiology, 37 (8): 1108-1115).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

In some embodiments of the invention, inducible promoters can be used. Thus, for example, chemical-regulated promoters can be used to modulate the expression of a gene in an organism through the application of an exogenous chemical regulator. Regulation of the expression of nucleotide sequences of the invention via promoters that are chemically regulated enables the nucleic acids and/or the polypeptides of the invention to be synthesized only when, for example, a crop of plants are treated with the inducing chemicals. Depending upon the objective, the promoter may be a chemical-inducible promoter, where application of a chemical induces gene expression, or a chemical-repressible promoter, where application of the chemical represses gene expression. In some aspects, a promoter can also include a light-inducible promoter, where application of specific wavelengths of light induces gene expression (Levskaya et al. 2005. Nature 438:441-442). In other aspects, a promoter can include a light-repressible promoter, where application of specific wavelengths of light repress gene expression (Ye et al. 2011. Science 332:1565-1568).

Chemically inducible promoters useful with plants are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1α promoter, which is activated by salicylic acid (e.g., the PR1a system), steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters.

In some embodiments, promoters useful with algae include, but are not limited to, the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)), the promoter of the σ⁷⁰-type plastid rRNA gene (Prrn), the promoter of the psbA gene (encoding the photosystem-II reaction center protein D1) (PpsbA), the promoter of the psbD gene (encoding the photosystem-II reaction center protein D2) (PpsbD), the promoter of the psaA gene (encoding an apoprotein of photosystem I) (PpsaA), the promoter of the ATPase alpha subunit gene (PatpA), and promoter of the RuBisCo large subunit gene (PrbcL), and any combination thereof (See, e.g., De Cosa et al. Nat. Biotechnol. 19:71-74 (2001); Daniell et al. BMC Biotechnol. 9:33 (2009); Muto et al. BMC Biotechnol. 9:26 (2009); Surzycki et al. Biologicals 37:133-138 (2009)).

In some embodiments, a promoter useful with this invention can include, but is not limited to, pol III promoters such as the human U6 small nuclear promoter (U6) and the human H1 promoter (H1) (Mäkinen et al. J Gene Med. 8 (4): 433-41 (2006)), and pol II promoters such as the CMV (Cytomegalovirus) promoter (Barrow et al. Methods in Mol. Biol. 329:283-294 (2006)), the SV40 (Simian Virus 40)-derived initial promoter, the EF-1α (Elongation Factor-1α) promoter, the Ubc (Human Ubiquitin C) promoter, the PGK (Murine Phosphoglycerate Kinase-1) promoter and/or constitutive protein gene promoters such as the β-actin gene promoter, the tRNA promoter and the like.

Moreover, tissue-specific regulated nucleic acids and/or promoters as well as tumor-specific regulated nucleic acids and/or promoters have been reported. Thus, in some embodiments, tissue-specific or tumor-specific promoters can be used. Some reported tissue-specific nucleic acids include, without limitation, B29 (B cells), CD14 (monocytic cells), CD43 (leukocytes and platelets), CD45 (hematopoietic cells), CD68 (macrophages), desmin (muscle), elastase-1 (pancreatic acinar cells), endoglin (endothelial cells), fibronectin (differentiating cells and healing tissues), FLT-1 (endothelial cells), GFAP (astrocytes), GPIIb (megakaryocytes), ICAM-2 (endothelial cells), INF-β (hematopoietic cells), Mb (muscle), NPHSI (podocytes), OG-2 (osteoblasts, SP-B (lungs), SYN1 (neurons), and WASP (hematopoietic cells). Some reported tumor-specific nucleic acids and promoters include, without limitation, AFP (hepatocellular carcinoma), CCKAR (pancreatic cancer), CEA (epithelial cancer), c-erbB2 (breast and pancreatic cancer), COX-2, CXCR4, E2F-1, HE4, LP, MUC1 (carcinoma), PRC1 (breast cancer), PSA (prostate cancer), RRM2 (breast cancer), survivin, TRP1 (melanoma), and TYR (melanoma).

In some embodiments, inducible promoters can be used. Examples of inducible promoters include, but are not limited to, tetracycline repressor system promoters, Lac repressor system promoters, copper-inducible system promoters, salicylate-inducible system promoters (e.g., the PR1a system), glucocorticoid-inducible promoters, and ecdysone-inducible system promoters.

In some embodiments, a promoter useful with the recombinant nucleic acid constructs of the invention may be a promoter from any bacterial species. In some embodiments, for example, a promoter from a Clostridium spp. may be operably linked to a recombinant nucleic acid construct of the invention (e.g., a CRISPR and/or a Cascade complex). In some embodiments, an endogenous promoter from Erysipelatoclostridium ramosum or Clostridium spp. 1141A1FAA may be operably linked to a recombinant nucleic acid construct of the invention. In some embodiments, a heterologous/exogenous promoter may be used.

In some embodiments, a promoter may be operably linked to a recombinant nucleic acid construct of the invention for expression in a bacterial cell (e.g., a clostridium cell (e.g. a cell of E. ramosum or Clostridium spp. 1141A1FAA)) or an archaeal cell. In some embodiments, a promoter may be operably linked to a recombinant nucleic acid construct of the invention for expression in a eukaryotic cell, including but not limited to a cell of an insect, a fungus, a plant, or an animal.

In some embodiments, a promoter or leader sequence useful with the invention includes, but is not limited to, those having the nucleotide sequences of SEQ ID NOs: 44-52.

In some embodiments of this invention, one or more terminators may be operably linked to a polynucleotide encoding a Cascade complex and or a Cas3 and/or a CRISPR of the invention. In some embodiments, a terminator sequence may be operably linked to the 3′ end of a terminal repeat in a CRISPR.

In some embodiments, when comprised in the same nucleic acid construct (e.g., expression cassette), each of the CRISPR and recombinant nucleic acid encoding a Cascade complex and/or a Cas3 polypeptide may be operably linked to separate (independent) terminators (that may be the same terminator or a different terminator) or to a single terminator. In some embodiments, only the CRISPR may be operably linked to a terminator. Thus, in some embodiments, a terminator sequence may be operably linked to the 3′ end of a CRISPR (e.g., linked to the 3′ end of the repeat sequence located at the 3′ end of the CRISPR).

Any terminator that is useful for defining the end of a transcriptional unit (such as the end of a CRISPR or a Cascade complex) and initiating the process of releasing the newly synthesized RNA from the transcription machinery may be used with this invention (e.g., an terminator that is functional with a polynucleotide comprising a CRISPR and/or a polynucleotide encoding a Cascade complex of the invention may be utilized (e.g., that can define the end of a transcriptional unit (such as the end of a CRISPR, Cas3 or Cascade complex) and initiate the process of releasing the newly synthesized RNA from the transcription machinery).

A non-limiting example of a terminator useful with this invention may be a Rho-independent terminator sequence. In some embodiments, a Rho-independent terminator sequence from L . . . crispatus may be the nucleotide sequence of (5′-3′)

(SEQ ID NO: 57)
AAAAAAAAACCCCGCCCCTGACAGGGCGGGGTTTTTTTT.

Further non-limiting examples of useful terminator sequences (5′-3′) include:

SEQ ID NO: 53
AAAAGATCCCGGATTCTGTATGATGCAGAGTCCGGGATTTTT;
SEQ ID NO: 54
GGAACCCCTGGCCAATATGGTCAGGGGTTCT;
SEQ ID NO: 55
ATGAATTGCAGAAATGCATTTCAGATATTTTTGAACCTTGAAAAC;
(SEQ ID NO: 58)
CAAAAAAAGCATGAGAATTAATTTTCTCATGCTTTTTTG;
(SEQ ID NO: 59)
AAAAAAGATGCACTTCTTCACAGGAGCGCATCTTTTTT;
(SEQ ID NO: 60)
CAAAAAGAGCGGCTATAGGCCGCTTTTTTTGC;
and/or
(SEQ ID NO: 61)
GTAAAAATGGCTTGCGTGTTGCAAGCCATTTTTTTAC.

In some embodiments, a recombinant nucleic acid construct of the invention may be an “expression cassette” or may be comprised within an expression cassette. As used herein, “expression cassette” means a recombinant nucleic acid construct comprising a polynucleotide of interest (e.g., a Cascade complex; Cas3) and/or a CRISPR of the invention, wherein said polynucleotide of interest and/or a CRISPR is operably associated with at least one control sequence (e.g., a promoter). Thus, some aspects of the invention provide expression cassettes designed to express the polynucleotides of the invention (e.g., the Cascade complexes; Cas3) and/or CRISPR of the invention.

An expression cassette comprising a nucleotide sequence of interest may be chimeric, meaning that at least one of its components is heterologous with respect to at least one of its other components. An expression cassette may also be one that is naturally occurring but has been obtained in a recombinant form useful for heterologous expression.

An expression cassette may also optionally include a transcriptional and/or translational termination region (i.e., termination region) that is functional in the selected host cell. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked polynucleotide of interest, may be native to the host cell, or may be derived from another source (i.e., foreign or heterologous to the promoter, to the polynucleotide of interest, to the host, or any combination thereof).

An expression cassette (e.g., recombinant nucleic acid construct(s) of the invention) may also include a nucleotide sequence for a selectable marker, which can be used to select a transformed host cell (e.g., force a cell to acquire and keep an introduced nucleic acid (e.g., expression cassette, vector (e.g., plasmid) comprising the recombinant nucleic acid constructs of the invention)). As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Of course, many examples of suitable selectable markers are known in the art and can be used in the expression cassettes described herein. In some embodiments, a selectable marker useful with this invention includes polynucleotide encoding a polypeptide conferring resistance to an antibiotic. Non-limiting examples of antibiotics useful with this invention include tetracycline, chloramphenicol, and/or erythromycin. Thus, in some embodiments, a polynucleotide encoding a gene for resistance to an antibiotic may be introduced into the organism, thereby conferring resistance to the antibiotic to that organism.

In addition to expression cassettes, the nucleic acid construct and nucleotide sequences described herein may be used in connection with vectors. The term “vector” refers to a composition for transferring, delivering or introducing a nucleic acid (or nucleic acids) into a cell. A vector comprises a nucleic acid construct comprising the nucleotide sequence(s) to be transferred, delivered or introduced. Vectors for use in transformation of host organisms are well known in the art. Non-limiting examples of general classes of vectors include but are not limited to a viral vector, a plasmid vector, a phage vector, a phagemid vector, a cosmid vector, a fosmid vector, a bacteriophage, an artificial chromosome, transposon, retrovirus or an Agrobacterium binary vector in double or single stranded linear or circular form which may or may not be self-transmissible or mobilizable. A vector as defined herein can transform a prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). A nucleic acid construct in the vector may be under the control of, and operably linked to, an appropriate promoter or other regulatory elements for transcription in a host cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell. Accordingly, the recombinant nucleic acid constructs of this invention and/or expression cassettes comprising the recombinant nucleic acid constructs of this invention may be comprised in vectors as described herein and as known in the art. In some embodiments, the constructs of the invention may be delivered in combination with polypeptides (e.g., Cascade complex polypeptides, Cas3) as ribonucleoprotein particles (RNPs). Thus, for example, a Cascade complex (or one or more polypeptides comprised in said Cascade complex) can be introduced as a DNA expression plasmid, e.g., in vitro transcripts, or as a recombinant protein bound to the RNA portion in a ribonucleoprotein particle (RNP) (e.g., protein-RNA complex), whereas the sgRNA can be delivered either expressed as a DNA plasmid or as an in vitro transcript.

Accordingly, in some embodiments, the invention provides a recombinant nucleic acid construct comprising a Clustered Regularly Interspaced Short Palindromic Repeats (CRISPR) comprising one or more repeat sequence(s) (e.g., 1, 2, 3, 4, 5, 6, 7, 8, 9, 10, 11, 12, 13, 14, 15 or more) and one or more spacer sequence(s) (e.g., 1, 2, 3, 4, 5, 6, 7, 8, 9, 10, 11, 12, 13, 14, 15 or more), wherein each spacer sequence and each repeat sequence have a 5′ end and a 3′ end and each spacer sequence is linked at its 5′ end and at its 3′ end to a repeat sequence, and the spacer sequence is complementary to a target sequence (protospacer) in a target DNA of a target organism that is located immediately adjacent (3′) to a protospacer adjacent motif (PAM). A CRISPR of the present invention comprises a minimum of two repeats, flanking a spacer, to be expressed as a premature CRISPR (pre-CRISPR, pre-crRNA) that will be processed internally in the cell to constitute the final mature CRISPR (crRNA).

In some embodiments, a repeat sequence (i.e., CRISPR repeat sequence) as used herein may comprise any known repeat sequence of a wild-type Clostridium CRISPR Type I-B locus (e.g., Clostridium spp. 1141A1FAA). In some embodiments, a repeat sequence useful with the invention may include a synthetic repeat sequence having a different nucleotide sequence than those known in the art for Clostridium but sharing similar structure to that of wild-type Clostridium repeat sequences of a hairpin structure with a loop region. Thus, in some embodiments, a repeat sequence may be identical to (i.e., having 100% sequence identity) or substantially identical (e.g., having 80% to 99% identity (e.g., about 80, 81, 82, 83, 84, 85, 86, 87, 88, 89, 90, 91, 92, 93, 94, 95, 96, 97, 98, 99% sequence identity)) to a repeat sequence from a wild-type Clostridium CRISPR Type I-B locus.

In some embodiments, a repeat sequence (i.e., CRISPR repeat sequence) as used herein may comprise any known repeat sequence of a wild-type Erysipelatoclostridium CRISPR Type I-B locus (e.g., E. ramosum). In some embodiments, a repeat sequence useful with the invention may include a synthetic repeat sequence having a different nucleotide sequence than those known in the art for Erysipelatoclostridium but sharing similar structure to that of wild-type Erysipelatoclostridium repeat sequences of a hairpin structure with a loop region. Thus, in some embodiments, a repeat sequence may be identical to (i.e., having 100% identity) or substantially identical (e.g., having about 80% to 99% identity (e.g., about 80, 81, 82, 83, 84, 85, 86, 87, 88, 89, 90, 91, 92, 93, 94, 95, 96, 97, 98, 99% identity)) to a repeat sequence from a wild-type Erysipelatoclostridium CRISPR Type I-B locus.

The length of a CRISPR repeat sequence useful with this invention may be the full length of a Clostridium spp. or Erysipelatoclostridium spp. (e.g., E. ramosum or C. spp. 1141A1FAA) repeat sequence (i.e., about 25 to 32 nucleotides) (see, e.g., SEQ ID NOs: 17, 18 or 35-43). In some embodiments, a repeat sequence may comprise a portion of a wild type Clostridium or Erysipelatoclostridium repeat nucleotide sequence, the portion being reduced in length by as much as 7 to 8 nucleotides (e.g., 1, 2, 3, 4, 5, 6, 7, or 8 nucleotides) from the 3′ end as compared to a wild type Clostridium or Erysipelatoclostridium repeat (e.g., comprising about 19 to 31 contiguous nucleotides from the 5′ end of a wild type Clostridium or Erysipelatoclostridium CRISPR Type I-B locus repeat sequence; e.g., about 19, 20, 20, 21, 22, 23, 24, 25, 26, 27, 28, 29, 30 or 31 contiguous nucleotides from the 5′ end, or any range or value therein). In some embodiments, a repeat sequence useful with this invention may comprise, consist essentially of or consist of at least 19 consecutive nucleotides (e.g., about 19, 20, 21, 22, 23, 24, 25, 26, 27, 28, 29, 30, 31, 32 or 33 consecutive nucleotides) having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the nucleotide sequences of SEQ ID NOs: 17, 18 or 35-43, optionally about 19, 20, 21, 22, 23, 24, or 25 consecutive nucleotides to about 26, 27, 28 29, 30, or 31 consecutive nucleotides, about 21, 22, 23, 24, or 25 consecutive nucleotides to about 26, 27, 28, 29, 30, 31, 32 or 33, or about 27 to about 32 consecutive nucleotides of the repeat sequences.

Thus, in some embodiments, a repeat sequence may comprise, consist essentially of, or consist of any of the nucleotide sequences of:

SEQ ID NO: 17
GTTTAATAACAACATAAGATGTATTGAAAT,
SEQ ID NO: 18
GTTTAATAACAACGAGATATTAAAGTGAAA,
SEQ ID NO: 35
GTTTCAATCCACGCACCCGTGCAGGGTGCGAC,
SEQ ID NO: 36
GTTTCAATTCCAACATGGTACGATTAAAGC,
SEQ ID NO: 37
GTTTCAATTCCAATATGGCACGATTAAAGC,
SEQ ID NO: 38
TGCTTTATTACAATGTGGTAAGAGTAAAGC,
SEQ ID NO: 39
CTTTCAATTCCAACTTGGTACGATTAAAAC,
SEQ ID NO: 40
AACGCAATTCCAACTTGGTACGATTAAAAC,
SEQ ID NO: 41
GTTTCAATTCCAACTTGGTACGATTAAAAC,
SEQ ID NO: 42
GTTGCAATTCCAATATGGTAAGATTAAAGC,
and/or
SEQ ID NO: 43
TCAATTCCAATATGGTACGATTAAAAC,
or
any combination thereof.

The bolded nucleotides indicate the nucleotides that differ from the consensus sequence

In some embodiments, when two or more repeat sequences are present in a CRISPR, they may comprise the same repeat sequence, may comprise different repeat sequences, or any combination thereof. In some embodiments, each of the two or more repeat sequences in single CRISPR may comprise, consist essentially of, or consist of the same repeat sequence.

A CRISPR useful with the methods of the invention may comprise one spacer sequence or more than one spacer sequence, wherein each spacer sequence is flanked by at least one repeat sequence (e.g., a repeat-spacer (non-natural) or a repeat-spacer-repeat), wherein the at least one repeat may be a full length repeat sequence or a portion thereof as described herein. In some embodiments, a CRISPR useful with this invention may comprise a spacer sequence linked at its 5′ end and its 3′ end to a repeat sequence (e.g., a repeat-spacer-repeat), wherein the repeat is a full length repeat sequence or a portion thereof. When more than one spacer sequence is present in a CRISPR of the invention, each spacer sequence is separated from the next spacer sequence by a repeat sequence. Thus, each spacer sequence is linked at the 3′ end and at the 5′ end to a repeat sequence. The repeat sequence that is linked to each end of the one or more spacers may be the same repeat sequence or it may be a different repeat sequence or any combination thereof.

In some embodiments, the one or more spacer sequences of the present invention may be about 25 nucleotides to about 60 nucleotides in length (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length, and any value or range therein). In some embodiments, a spacer sequence may be a length of about 25 nucleotides to about 45 nucleotides (e.g., about 25, 26, 27, 28, 29, 30, 31, 32, 33, 34, 35, 36, 37, 38, 39, 40, 41, 42, 43, 44, or 45 nucleotides in length, and any value or range therein), about 30 nucleotides to about 40 nucleotides (e.g., about 30, 31, 32, 33, 34, 35, 36, 37, 38, 39, or 40 nucleotides in length, and any value or range therein) or about 32 nucleotides to about 40 nucleotides (e.g., about 30, 31, 32, 33, 34, 35, 36, 37, 38, 39, or 40 nucleotides in length, and any value or range therein). In some embodiments, a spacer sequence may comprise, consist essentially of, or consist of a length of about 34, 35, 36 or 37 nucleotides in length. In some embodiments, a spacer sequence may comprise, consist essentially of, or consist of a length of about 34 nucleotides in length. In some embodiments, a spacer sequence may comprise, consist essentially of, or consist of a length of about 35 nucleotides in length. In some embodiments, a spacer sequence may comprise, consist essentially of, or consist of a length of about 36 nucleotides in length. In some embodiments, a spacer sequence may comprise, consist essentially of, or consist of a length of about 37 nucleotides in length.

A spacer sequence may be fully complementary to a target sequence (e.g., 100% complementary to a target sequence across its full length). In some embodiments, a spacer sequence may be substantially complementary (e.g., at least about 80% complementary (e.g., about 80%, 81%, 82%, 83%, 84%, 85%, 86%, 87%, 88%, 89%, 90%, 91%, 92%, 93%, 94%, 95%, 96%, 97%, 98%, 98.5%, 99%, 99.5%, or more complementary)) to a target sequence from a target genome. Thus, in some embodiments, a spacer sequence may have one, two, three, four, five or more mismatches that may be contiguous or noncontiguous as compared to a target sequence from a target genome. In some embodiments, a spacer sequence may be about 80% to 100% (e.g., about 80%, 81%, 82%, 83%, 84%, 85%, 86%, 87%, 88%, 89%, 90%, 91%, 92%, 93%, 94%, 95%, 96%, 97%, 98%, 99%, or 100%) complementary to a target sequence from a target genome. In some embodiments, a spacer sequence may be about 85% to 100% (e.g., about 85%, 86%, 87%, 88%, 89%, 90%, 91%, 92%, 93%, 94%, 95%, 96%, 97%, 98%, 99%, or 100%)) complementary to a target sequence from a target genome. In some embodiments, a spacer sequence may be about 90% to 100% (e.g., about 90%, 91%, 92%, 93%, 94%, 95%, 96%, 97%, 98%, 99%, or 100%) or about 95% to 100% (e.g., about 95%, 96%, 97%, 98%, 98.5%, 99%, 99.5% or 100%) complementary to a target sequence from a target genome.

In some embodiments, the 5′ region of a spacer sequence may be fully complementary to a target sequence while the 3′ region of the spacer sequence may be substantially complementary to the target sequence. Accordingly, in some embodiments, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) may be about 100% complementary to a target sequence, while the remainder of the spacer sequence may be about 80% or more complementary to the target sequence.

In some embodiments, at least the first eight contiguous nucleotides at the 5′ end of a spacer sequence of the invention are fully complementary to the portion of the target sequence adjacent to the PAM (termed a “seed sequence”). Thus, in some embodiments, the seed sequence may comprise the first 8 nucleotides of the 5′ end of each of one or more spacer sequence(s), which first 8 nucleotides are fully complementary (100%) to the target sequence, and the remaining portion of the one or more spacer sequence(s) (3′ to the seed sequence) may be at least about 80% complementarity (e.g., about 80, 81, 82, 83, 84, 85, 86, 87, 88, 89, 90, 91, 92, 93, 94, 95, 96, 97, 98, 99, or 100% complementarity) to the target sequence. Thus, for example, a spacer sequence having a length of 28 nucleotides may comprise a seed sequence of eight contiguous nucleotides located at the 5′ end of the spacer sequence, which is 100% complementary to the target sequence, while the remaining 20 nucleotides may be about 80% to about 100% complimentary to the target sequence (e.g., 0 to 4 non-complementary nucleotides out of the remaining 20 in the spacer sequence or 0 to 2 non-complementary nucleotides out of the remaining 12 nucleotides in the spacer sequence). As another example, a spacer sequence having a length of 33 nucleotides may comprise a seed sequence of eight nucleotides from the 5′ end, which is 100% complementary to the target sequence, while the remaining 25 nucleotides may be at least about 80% (e.g., 0 to 5 non-complementary nucleotides out of the remaining 25 nucleotides in the spacer sequence) or a spacer sequence having a length of 37 nucleotides may comprise a seed sequence of eight nucleotides from the 5′ end, which is 100% complementary to the target sequence, while the remaining 29 nucleotides may be at least about 80% (e.g., 0 to 5 non-complementary nucleotides out of the remaining 29 nucleotides in the spacer sequence). In a further example, a spacer sequence having a length of 34 nucleotides may comprise a seed sequence of eight nucleotides from the 5′ end, which is 100% complementary to the target sequence, while the remaining 26 nucleotides may be at least about 80% (e.g., 0 to 5 non-complementary nucleotides out of the remaining 26 nucleotides in the spacer sequence) or a spacer sequence having a length of 38 nucleotides may comprise a seed sequence of eight nucleotides from the 5′ end, which is 100% complementary to the target sequence, while the remaining 30 nucleotides may be at least about 80% (e.g., 0 to 6 non-complementary nucleotides out of the remaining 30 nucleotides in the spacer sequence).

A CRISPR useful with the invention comprising more than one spacer sequence may be designed to target one or more than one target sequence (protospacer). Thus, in some embodiments, when a recombinant nucleic acid construct of the invention comprises a CRISPR that comprises at least two spacer sequences, the at least two spacer sequences may be complementary to two or more different target sequences. In some embodiments, when a recombinant nucleic acid construct of the invention comprises a CRISPR that comprises at least two spacer sequences, the at least two spacer sequences may be complementary to the same target sequence. In some embodiments, a CRISPR comprising at least two spacer sequences, the at least two spacer sequences may be complementary to different portions of one gene.

In some embodiments, a recombinant nucleic acid construct of the invention may encode a Type I-B CRISPR associated complex for antiviral defense complex (Cascade complex) comprising: (1) a Cas6 polypeptide, a Cas8 polypeptide, a Cas7 polypeptide, and a Cas5 polypeptide. In some embodiments, a recombinant nucleic acid construct of the invention may further comprise a Cas3 polypeptide of a Type I-B CRISPR-Cas system.

In some embodiments, a Cas6 polypeptide comprises the amino acid sequence of SEQ ID NO:1 or SEQ ID NO:19 (or a polypeptide sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity to the amino acid sequence of SEQ ID NO: 1 or SEQ ID NO:19). In some embodiments, a Cas8 polypeptide comprises the amino acid sequence of SEQ ID NO:2 or SEQ ID NO:20 (or a polypeptide sequence having at least 80% sequence identity to the amino acid sequence of SEQ ID NO:2 or SEQ ID NO:20). In some embodiments, a Cas7 polypeptide comprises the amino acid sequence of SEQ ID NO:3 or SEQ ID NO:21 (or a polypeptide sequence having at least 80% sequence identity to any one of the amino acid sequence of SEQ ID NO:3 or SEQ ID NO:21). In some embodiments, a Cas5 polypeptide comprises a sequence having at least 80% sequence identity to the amino acid sequence of SEQ ID NO:4 or SEQ ID NO:22 (or a polypeptide sequence having at least 80% sequence identity to any one of the amino acid sequence of SEQ ID NO:4 or SEQ ID NO:22). In some embodiments, a Cas3 polypeptide comprises the amino acid sequence of SEQ ID NO:5 or SEQ ID 23 (or a polypeptide sequence having at least 80% sequence identity to the amino acid sequence of SEQ ID NO:5 or SEQ ID NO:23).

In some embodiments, a Cas6 polypeptide is encoded by a nucleotide sequence of SEQ ID NO:9 or SEQ ID NO:27 or a nucleotide sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to SEQ ID NO:9 or SEQ ID NO:27. In some embodiments, a Cas8 polypeptide is encoded by the nucleotide sequence of SEQ ID NO: 10 or SEQ ID NO:28 or a nucleotide sequence having at least 80% sequence identity to SEQ ID NO: 10 or SEQ ID NO:28. In some embodiments, a Cas7 polypeptide is encoded by the nucleotide sequence of SEQ ID NO:11 or SEQ ID NO:29 or a nucleotide sequence having at least 80% sequence identity to SEQ ID NO: 11 or SEQ ID NO:29. In some embodiments, a Cas5 polypeptide is encoded by the nucleotide sequence of SEQ ID NO:12 or SEQ ID NO:30 or a nucleotide sequence having at least 80% sequence identity to SEQ ID NO:12 or SEQ ID NO:30. In some embodiments, a Cas3 polypeptide is encoded by the nucleotide sequence of SEQ ID NO:13 or SEQ ID NO:31 or a nucleotide sequence having at least 80% sequence identity to SEQ ID NO: 13 or SEQ ID NO:31.

Accordingly, in some embodiments, the present invention provides recombinant nucleic acid molecules encoding one or more polypeptides of a Cascade complex, the one or more polypeptides of a Cascade complex comprising a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1 or encoded by a nucleotide sequence having at least 80% sequence identity to SEQ ID NO:9, a Cas8 polypeptide having at least 80% sequence identity to the amino acid sequence of SEQ ID NO:2 or encoded by a nucleotide sequence having at least 80% sequence identity to SEQ ID NO:10, a Cas7 polypeptide having at least 80% sequence identity to the amino acid sequence of SEQ ID NO:3 or encoded by a nucleotide sequence having at least 80% sequence identity to SEQ ID NO:11, and a Cas5 polypeptide having at least 80% sequence identity to the amino acid sequence of SEQ ID NO:4 or encoded by a nucleotide sequence having at least 80% sequence identity to SEQ ID NO:12, optionally, wherein when used in combination with a CRISPR, the CRISPR comprises one or more repeat sequences, or portion thereof, having at least 80% sequence identity to a nucleotide sequence of SEQ ID NO: 17 or SEQ ID NO: 18, or any combination thereof.

In some embodiments, the one or more polypeptides of a Cascade complex comprising a Cas6 polypeptide having at least 80% sequence identity to SEQ ID NO:1 or encoded by a nucleotide sequence having at least 80% sequence identity to SEQ ID NO:9 or a Cas8 polypeptide comprising an amino acid sequence having at least 80% sequence identity to the amino acid sequence of SEQ ID NO:2 or encoded by a nucleotide sequence having at least 80% sequence identity to SEQ ID NO:10, a Cas7 polypeptide having at least 80% sequence identity to the amino acid sequence of SEQ ID NO:3 or encoded by a nucleotide sequence having at least 80% sequence identity to SEQ ID NO:11, and a Cas5 polypeptide having at least 80% sequence identity to the amino acid sequence of SEQ ID NO:4 or encoded by a nucleotide sequence having at least 80% sequence identity to SEQ ID NO:12, and a Cas3 polypeptide having at least 80% sequence identity to the amino acid sequence of SEQ ID NO:5 or encoded by a nucleotide sequence having at least 80% sequence identity to SEQ ID NO:13, optionally, wherein when used in combination with a CRISPR, the CRISPR comprises one or more repeat sequences, or portion thereof, having at least 80% sequence identity to a nucleotide sequence of SEQ ID NO: 17 or SEQ ID NO:18, or any combination thereof.

In some embodiments, the present invention provides recombinant nucleic acid molecules encoding one or more polypeptides of a Cascade complex, the one or more polypeptides of a Cascade complex comprising a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to SEQ ID NO: 19 or encoded by the nucleotide sequence of SEQ ID NO:27, a Cas8 polypeptide having at least 80% sequence identity to SEQ ID NO:20 or encoded by a nucleotide sequence having at least 80% sequence identity to SEQ ID NO:28, a Cas7 polypeptide having at least 80% sequence identity to SEQ ID NO:21 or encoded by a nucleotide sequence having at least 80% sequence identity to SEQ ID NO:29, and a Cas5 polypeptide having at least 80% sequence identity to SEQ ID NO:22 or encoded by a nucleotide sequence having at least 80% sequence identity to SEQ ID NO:30, optionally wherein when used in combination with a CRISPR, the CRISPR comprises one or more repeat sequences, or portion thereof, having at least 80% sequence identity to the nucleotide sequence of SEQ ID NOs: 35-43 or any combination thereof.

In some embodiments, recombinant nucleic acid molecules encoding one or more polypeptides of a Cascade complex are provided wherein the one or more polypeptides of a Cascade complex comprise a Cas6 polypeptide having at least 80% sequence identity to SEQ ID NO: 19 or encoded by a nucleotide sequence having at least 80% sequence identity to SEQ ID NO:27, a Cas8 polypeptide having at least 80% sequence identity to SEQ ID NO: 20 or encoded by a nucleotide sequence having at least 80% sequence identity to SEQ ID NO: 28, a Cas7 polypeptide having at least 80% sequence identity to SEQ ID NO:21 or encoded by a nucleotide sequence having at least 80% sequence identity to SEQ ID NO:29, and a Cas5 polypeptide having at least 80% sequence identity to SEQ ID NO:22 or encoded by a nucleotide sequence having at least 80% sequence identity to SEQ ID NO:30, and a Cas3 polypeptide having at least 80% sequence identity to SEQ ID NO:23 or encoded by a nucleotide sequence having at least 80% sequence identity to SEQ ID NO:31, optionally wherein when used in combination with a CRISPR, the CRISPR comprises one or more repeat sequences, or portion thereof, having at least 80% sequence identity to the nucleotide sequence of SEQ ID NOs: 35-43 or any combination thereof.

In some embodiments, the invention provides a CRISPR and the polypeptides of the Cascade complex and optionally a Cas3 in a protein-RNA complex (ribonucleoprotein, RNP). Thus, in some embodiments a protein-RNA complex is provided comprising: (a) a Type I-B CRISPR associated complex for antiviral defense complex (Cascade complex) comprising (i) a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1, a Cas8 polypeptide having at least 80% sequence identity to the amino acid sequence of SEQ ID NO:2, a Cas7 polypeptide having at least 80% sequence identity to the amino acid sequence of SEQ ID NO:3, a Cas5 polypeptide having at least 80% sequence identity to the amino acid sequence of SEQ ID NO:4, and a Cas3 polypeptide having at least 80% sequence identity to the amino acid sequence of SEQ ID NO:5; or (ii) a Cas6 polypeptide having at least 80% sequence identity to the amino acid sequence of SEQ ID NO: 19, a Cas8 polypeptide having at least 80% sequence identity to the amino acid sequence of SEQ ID NO:20, a Cas7 polypeptide having at least 80% sequence identity to the amino acid sequence of SEQ ID NO:21, a Cas5 polypeptide having at least 80% sequence identity to the amino acid sequence of SEQ ID NO:22, and a Cas3 polypeptide having at least 80% sequence identity to the amino acid sequence of SEQ ID NO:23; and (b) a Clustered Regularly Interspaced Short Palindromic Repeats (CRISPR) comprising one or more repeat sequences and one or more spacer sequence(s), wherein each spacer sequence is linked at its 5′ end and at its 3′ end to a repeat sequence, and the spacer sequence is complementary to a target sequence (protospacer) in a nucleic acid of a target organism, wherein the target sequence is located immediately adjacent (3′) to a protospacer adjacent motif (PAM).

In contrast to the recombinant nucleic acid constructs and protein-RNA constructs of the present invention, a wild type Type I-B Cascade complex of Erysipelatoclostridium ramosum further comprises Cas4, Cas1 and Cas2 (see, polypeptide sequences of SEQ ID NOs: 6, 7 and 8, respectively; nucleotide sequences of SEQ ID NOs: 14, 15, 16), which are responsible for spacer acquisition in wild type CRISPR-Cas systems. Similarly, a wild type Type I-B Cascade complex of Clostridium spp. 1141A1FAA further comprises Cas4, Cas1 and Cas2 (see, polypeptide sequences of SEQ ID NOs: 24, 25 and 26, respectively; nucleotide sequences of SEQ ID NOs: 32, 33, 34), which are responsible for spacer acquisition in wild type CRISPR-Cas systems.

In some embodiments, the recombinant nucleic acid constructs of the invention may be comprised in a vector (e.g., a plasmid, a phagemid, a transposon, a bacteriophage, and/or a retrovirus. Thus, in some embodiments, the invention further provides phagemid, plasmid, bacteriophage, transposon, and/or retroviral vectors comprising the recombinant nucleic acid constructs of the invention.

Plasmids useful with the invention may be dependent on the target organism, that is, dependent on where the plasmid is to replicate. Non-limiting examples of plasmids that express in Lactobacillus include pNZ and derivatives, pGK12 and derivatives, pTRK687 and derivatives, pTRK563 and derivatives, pTRKH2 and derivatives, pIL252, and/or pIL253. Additional, non-limiting plasmids of interest include pORI-based plasmids or other derivatives and homologs.

Accordingly, the present invention provides one vector or more than one vector encoding a recombinant nucleic acid of the invention. In some embodiments, vector may comprise, consist essentially of or consist or a recombinant nucleic acid encoding a Type I-B CRISPR associated complex for antiviral defense complex (Cascade complex) comprising (1) a Cas6 polypeptide, a Cas8 polypeptide, a Cas7 polypeptide, and a Cas5 polypeptide; and/or (2) a Cas6 polypeptide, a Cas8 polypeptide, a Cas7 polypeptide, a Cas5 polypeptide, and a Cas3 polypeptide, wherein the Cas6 polypeptide comprises the amino acid sequence of SEQ ID NO: 1, the Cas8 polypeptide comprises the amino acid sequence of SEQ ID NO:2, the Cas7 polypeptide comprises the amino acid sequence of SEQ ID NO:3, the Cas5 polypeptide comprises the amino acid sequence of SEQ ID NO:4, and the Cas3 polypeptide comprises the amino acid sequence of SEQ ID NO:5; or the Cas6 polypeptide comprises the amino acid sequence of SEQ ID NO:19, the Cas8 polypeptide comprises the amino acid sequence of SEQ ID NO:20, the Cas7 polypeptide comprising the amino acid sequence of SEQ ID NO: 21, the Cas5 polypeptide comprising the amino acid sequence of SEQ ID NO:22, and the Cas3 polypeptide comprises the amino acid sequence of SEQ ID NO:23.

The compositions (e.g., recombinant nucleic acid constructs) of the present invention may be used, for example, in methods for modifying nucleic acids such as modifying the genome of a target organism or a cell thereof, in methods for selection of variants in a population or for selected killing of cells in a population. In some embodiments, the nucleic acid modification may be carried out in a cell free system. In some embodiments, the nucleic acid or genome modification may be directed to targeted gene silencing, repression of expression and/or modulation of the repression of expression in an organism of interest or cell thereof or in a cell free system.

For use in such methods, the recombinant nucleic acid constructs of the invention may be introduced into a cell of an organism, or where relevant, the constructs may be contacted with a target nucleic acid in a cell free system. In some embodiments, the recombinant nucleic acid constructs of the invention may be stably or transiently introduced into a cell of an organism of interest for the purpose of modifying the genome and/or for altering expression in the cell or for modifying the target nucleic acid or its expression in the cell free system.

Accordingly, in some embodiments, a method of modifying (editing) the genome of a target organism, comprising introducing into the target organism or a cell of the target organism (a) a recombinant nucleic acid construct comprising a Clustered Regularly Interspaced Short Palindromic Repeats (CRISPR) comprising one or more repeat sequences and one or more spacer sequence(s), wherein each spacer sequence is linked at its 5′ end and at its 3′ end to a repeat sequence, and the spacer sequence is complementary to a target sequence (protospacer) in a target nucleic acid of a target organism, wherein the target sequence is located immediately adjacent (3′) to a protospacer adjacent motif (PAM); (b) a recombinant nucleic acid construct encoding a Type I-B CRISPR associated complex for antiviral defense complex (Cascade complex) comprising: (i) a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1, a Cas8 polypeptide having at least 80% sequence identity to the amino acid sequence of SEQ ID NO:2, a Cas7 polypeptide having at least 80% sequence identity to the amino acid sequence of SEQ ID NO: 3, a Cas5 polypeptide having at least 80% sequence identity to the amino acid sequence of SEQ ID NO:4, or a Cas3 polypeptide having at least 80% sequence identity to the amino acid sequence of SEQ ID NO:5; or (ii) a Cas6 polypeptide having at least 80% sequence identity to the amino acid sequence of SEQ ID NO:19, a Cas8 polypeptide having at least 80% sequence identity to the amino acid sequence of SEQ ID NO:20, a Cas7 polypeptide having at least 80% sequence identity to the amino acid sequence of SEQ ID NO:21, a Cas5 polypeptide having at least 80% sequence identity to the amino acid sequence of SEQ ID NO: 22, or a Cas3 polypeptide having at least 80% sequence identity to the amino acid sequence of SEQ ID NO:23; and (c) a repair template, thereby modifying the genome of the target organism.

In some embodiments, when a cell or organism of interest comprises an endogenous CRISPR-Cas system that is compatible with the recombinant CRISPRs of the invention (e.g., a Type I-B CRISPR Cas system; e.g., a Type I-B CRISPR Cas system of E. ramosum or Clostridium spp. 1141A1FAA), the endogenous CRISPR-Cas system of a cell (e. g., endogenous Cascade complex) may be co-opted for use with the recombinant CRISPRs of the invention (e.g., the recombinant nucleic acid constructs comprising a CRISPR) for the purpose of modifying the genome and/or for altering expression in the cell. In some embodiments, the target organism is a prokaryote or a eukaryote. In some embodiments, the target organism is a bacterial cell that is from a commensal bacterial species or strain, optionally the bacterial cell is a commensal Clostridium spp. or strain.

Accordingly, in some embodiments, the present invention provides a method of modifying (editing) the genome of a bacterial cell comprising a Type I-B CRISPR-Cas system that is compatible with the recombinant constructs of the invention, comprising introducing into the bacterial cell (a) a recombinant nucleic acid construct comprising a Clustered Regularly Interspaced Short Palindromic Repeats (CRISPR) comprising one or more repeat sequences and one or more spacer nucleotide sequence(s), wherein each of the one or more spacer sequences comprises a 3′ end and a 5′ end and is linked at its 5′ end and at its 3′ end to a repeat sequence, and each of the one or more spacer sequences is complementary to a target sequence (protospacer) in the genome of the bacterial cell, wherein the target sequence is located immediately adjacent (3′) to a protospacer adjacent motif (PAM); and (b) a repair template, thereby modifying the genome of the bacterial cell. In some embodiments, the bacterial cell is a cell of a commensal bacterial species, optionally the bacterial is a commensal Clostridium spp.

A CRISPR of the invention may also be introduced into a cell (or cell free environment) in the form of a protein-RNA complex (RNP). Thus, in some embodiments, the invention provides a method of modifying (editing) the genome of a target organism, comprising introducing into the target organism or a cell of the target organism a protein-RNA complex, the protein-RNA complex comprising: (a) a Clustered Regularly Interspaced Short Palindromic Repeats (CRISPR) comprising one or more repeat sequences and one or more spacer sequence(s), wherein each spacer sequence is linked at its 5′ end and at its 3′ end to a repeat sequence, and the spacer sequence is complementary to a target sequence (protospacer) in a target nucleic acid of a target organism, wherein the target sequence is located immediately adjacent (3′) to a protospacer adjacent motif (PAM); (b) a Type I-B CRISPR associated complex for antiviral defense complex (Cascade complex) comprising (i) a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1, a Cas8 polypeptide having at least 80% sequence identity to the amino acid sequence of SEQ ID NO: 2, a Cas7 polypeptide having at least 80% sequence identity to the amino acid sequence of SEQ ID NO:3, a Cas5 polypeptide having at least 80% sequence identity to the amino acid sequence of SEQ ID NO:4, and a Cas3 polypeptide having at least 80% sequence identity to the amino acid sequence of SEQ ID NO:5; or (ii) a Cas6 polypeptide having at least 80% sequence identity to the amino acid sequence of SEQ ID NO:19, a Cas8 polypeptide having at least 80% sequence identity to the amino acid sequence of SEQ ID NO: 20, a Cas7 polypeptide having at least 80% sequence identity to the amino acid sequence of SEQ ID NO:21, a Cas5 polypeptide having at least 80% sequence identity to the amino acid sequence of SEQ ID NO:22, and a Cas3 polypeptide having at least 80% sequence identity to the amino acid sequence of SEQ ID NO:23; and (c) a repair template, thereby modifying the genome of the target organism.

A “repair template” may be any template DNA that is useful for introducing a desired modification into a target nucleic acid. The repair template may be engineered to generate a deletion, an insertion, a single base mutation, and span various sizes (adding or removing one base, or adding or removing a whole gene or even operon). In some embodiments, for generation of a deletion, a repair template is designed that contains homologous arms to the chromosomal region adjacent to the region to delete, but not including the sequences of the region to delete. In some embodiments, for generation of an insertion, a repair template is designed that contains homologous arms to the chromosomal region adjacent to the insertion point and the sequence to insert. In some embodiments, for generation of a single nucleotide substitution, a repair template is designed that contains homologous arms to the chromosomal region to modify including the sequence alteration. In some embodiments, an engineered single-stranded DNA (ssDNA) sequence (e.g. oligonucleotide) containing a polynucleotide of interest to be altered in the chromosome, can be used for recombineering purposes.

In some embodiments, the present invention further provides a method of altering the expression (repressing expression/overexpression) of a target gene in a target organism, comprising introducing into the target organism or a cell of the target organism (a) a recombinant nucleic acid construct comprising a Clustered Regularly Interspaced Short Palindromic Repeats (CRISPR) comprising one or more (e.g., 1, 2, 3, 4, 5, 6, 7, 8, 9, 10, 11, 12, 13, 14, 15 or more) repeat sequence(s) and one or more (e.g., 1, 2, 3, 4, 5, 6, 7, 8, 9, 10, 11, 12, 13, 14, 15 or more) spacer sequence(s), wherein each spacer sequence is linked at its 5′ end and at its 3′ end to a repeat sequence, and the spacer sequence is complementary to a target sequence (protospacer) in a nucleic acid of a target organism, wherein the target sequence is located immediately adjacent (3′) to a protospacer adjacent motif (PAM); (b) a recombinant nucleic acid construct encoding a Type I-B CRISPR associated complex for antiviral defense complex (Cascade complex) comprising: (i) a Cas6 polypeptide having at least 80% sequence identity to the amino acid sequence of SEQ ID NO:1, a Cas8 polypeptide having at least 80% sequence identity to the amino acid sequence of SEQ ID NO: 2, a Cas7 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO: 3, and a Cas5 polypeptide having at least 80% sequence identity to the amino acid sequence of SEQ ID NO:4; or (ii) a Cas6 polypeptide having at least 80% sequence identity to the amino acid sequence of SEQ ID NO:19, a Cas8 polypeptide having at least 80% sequence identity to the amino acid sequence of SEQ ID NO:20, a Cas7 polypeptide having at least 80% sequence identity to the amino acid sequence of SEQ ID NO:21, and a Cas5 polypeptide having at least 80% sequence identity to the amino acid sequence of SEQ ID NO: 22, thereby altering expression of the target gene in the cell of the target organism.

In some embodiments, a method of altering the expression (repressing expression/overexpression) of a target gene in a target organism, may comprise introducing into the target organism or a cell of the target organism a protein-RNA complex, the protein-RNA complex comprising: (a) a Clustered Regularly Interspaced Short Palindromic Repeats (CRISPR) comprising one or more (e.g., 1, 2, 3, 4, 5, 6, 7, 8, 9, 10, 11, 12, 13, 14, 15 or more) repeat sequence(s) and one or more (e.g., 1, 2, 3, 4, 5, 6, 7, 8, 9, 10, 11, 12, 13, 14, 15 or more) spacer sequence(s), wherein each spacer sequence is linked at its 5′ end and at its 3′ end to a repeat sequence, and the spacer sequence is complementary to a target sequence (protospacer) in a target nucleic acid of a target organism, wherein the target sequence is located immediately adjacent (3′) to a protospacer adjacent motif (PAM); and (b) a Type I-B CRISPR associated complex for antiviral defense complex (Cascade complex) comprising (i) a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1, a Cas8 polypeptide having at least 80% sequence identity to the amino acid sequence of SEQ ID NO: 2, a Cas7 polypeptide having at least 80% sequence identity to the amino acid sequence of SEQ ID NO:3, and a Cas5 polypeptide having at least 80% sequence identity to the amino acid sequence of SEQ ID NO:4; or (ii) a Cas6 polypeptide having at least 80% sequence identity to the amino acid sequence of SEQ ID NO:19, a Cas8 polypeptide having at least 80% sequence identity to the amino acid sequence of SEQ ID NO:20, a Cas7 polypeptide having at least 80% sequence identity to the amino acid sequence of SEQ ID NO: 21, and a Cas5 polypeptide having at least 80% sequence identity to the amino acid sequence of SEQ ID NO:22, thereby altering expression of the target gene in the cell of the target organism.

“Altering of expression” or “modifying expression” refers to, for example, the repression of expression, or the overexpression (e.g., increased expression), of a gene or genes.

In some embodiments, the methods of the present invention provide increased expression. In some embodiments, the methods of the present invention provide expression or increased expression as compared to a control (e.g., a cell in which the recombinant constructs of the invention are not introduced) (e.g., an increase of 5, 10, 15, 20, 25, 30, 35, 40, 45, 50, 55, 60, 65, 70, 75, 80, 85, 90, 95, 100% or more, as compared to a control). Thus, for example, tethering a Cascade complex to a repressor factor may release the corresponding gene from repression, resulting in its expression or increased expression as compared to a control. In some embodiments, tethering a Cascade complex to an activator (e.g., promoter) may also result in expression or increased expression of the operably linked gene(s).

In some embodiments, the methods of the present invention provide reduced expression (e.g., repression of expression). Repression of expression can occur when tethering a Cascade complex to a gene, thereby prevention transcription and reducing expression as compared to a control (e.g., a reduction of about 5, 10, 15, 20, 25, 30, 35, 40, 45, 50, 55, 60, 65, 70, 75, 80, 85, 90, 95, 100% as compared to a control). In a further non-limiting example, repression of expression can be accomplished by tethering the Cascade complex to a repressor.

In some embodiments, the level of repression or induction of expression may be over a log 10 scale (e.g., about 5× to about 100,000×) (e.g., about 5×, 10×, 25×, 50×, 75×, 100×, 125×, 150×, 175×, 200×, 300×, 400×, 500×, 600×, 700×, 800×, 900×, 1000×, 2000×, 3000×, 4000×, 5000×, 6000×, 7000×, 8000×, 9000×, 10000×, and the like, and any value or range therein).

In some embodiments, the present invention further provides a method of screening for a variant cell of an organism, the method comprising (a) introducing into a population of cells from (or of) the organism: (i) a recombinant nucleic acid construct comprising a Clustered Regularly Interspaced Short Palindromic Repeats (CRISPR) comprising one or more (e.g., 1, 2, 3, 4, 5, 6, 7, 8, 9, 10 or more) repeat sequence(s) and one or more (e.g., 1, 2, 3, 4, 5, 6, 7, 8, 9, 10 or more) spacer sequence(s), wherein each spacer sequence is linked at its 5′ end and at its 3′ end to a repeat sequence, and the spacer sequence is complementary to a target sequence (protospacer) in a target nucleic acid (i.e., DNA) of at least a portion of the population of cells of the organism, wherein the target sequence is not present in the variant cell and the target sequence is located immediately adjacent (3′) to a protospacer adjacent motif (PAM); (ii) a recombinant nucleic acid construct encoding a Type I-B CRISPR associated complex for antiviral defense complex (Cascade complex) comprising: A) a Cas6 polypeptide having at least 80% sequence identity to the amino acid sequence of SEQ ID NO: 1, a Cas8 polypeptide having at least 80% sequence identity to the amino acid sequence of SEQ ID NO:2, a Cas7 polypeptide having at least 80% sequence identity to the amino acid sequence of SEQ ID NO:3, a Cas5 polypeptide having at least 80% sequence identity to the amino acid sequence of SEQ ID NO:4, or a Cas3 polypeptide having at least 80% sequence identity to the amino acid sequence of SEQ ID NO:5; or B) a Cas6 polypeptide having at least 80% sequence identity to the amino acid sequence of SEQ ID NO:19, a Cas8 polypeptide having at least 80% sequence identity to the amino acid sequence of SEQ ID NO: 20, a Cas7 polypeptide having at least 80% sequence identity to the amino acid sequence of SEQ ID NO:21, a Cas5 polypeptide having at least 80% sequence identity to the amino acid sequence of SEQ ID NO:22, or a Cas3 polypeptide having at least 80% sequence identity to the amino acid sequence of SEQ ID NO:23; wherein the recombinant nucleic acid construct comprising a CRISPR and the recombinant nucleic acid construct encoding a Cascade complex each comprise a polynucleotide encoding a polypeptide conferring resistance to a selection agent, thereby killing transformed cells comprising the target sequence and producing a subpopulation of cells of the population of cells; and (b) selecting from the subpopulation of cells produced in (a) one or more cells that are resistant to the selection agent, thereby selecting from the population of cells one or more variant cells (e.g., a subpopulation of cells) that do not comprise the target sequence and are not killed (e.g., the target sequence has been lost from or mutated in cells of the population that are not killed).

Further provided herein is a method of screening for variant bacterial cells that comprise an endogenous Type I-B CRISPR-Cas system, the method comprising (a) introducing into a population of bacterial cells a recombinant nucleic acid construct comprising a Clustered Regularly Interspaced Short Palindromic Repeats (CRISPR) comprising one or more (e.g., 1, 2, 3, 4, 5, 6, 7, 8, 9, 10, 11, 12, 13, 14, 15 or more) repeat sequence(s) and one or more (e.g., 1, 2, 3, 4, 5, 6, 7, 8, 9, 10, 11, 12, 13, 14, 15 or more) spacer sequence(s), wherein each spacer sequence is linked at its 5′ end and at its 3′ end to a repeat sequence, and the spacer sequence is complementary to a target sequence (protospacer) in a nucleic acid of the bacterial cells, wherein the target sequence is not present in the variant cell and wherein the target sequence is located immediately adjacent (3′) to a protospacer adjacent motif (PAM); and wherein the recombinant nucleic acid construct comprising a CRISPR comprises a polynucleotide encoding a polypeptide conferring resistance to a selection agent, thereby killing transformed cells comprising the target sequence and producing a subpopulation of bacterial cells that are not killed (by the selection agent or the CRISPR-Cas machinery); and (b) selecting from the subpopulation of bacterial cells produced in (a) one or more bacterial cells that are resistant to the selection agent, thereby selecting one or more variant bacterial cells that do not comprise the target sequence and are not killed (e.g., the target sequence has been lost from or mutated in cells of the population that are not killed). In some embodiments, the population of bacterial cells is a population of commensal Clostridium cells.

In some embodiments, a method of screening for a variant cell of an organism, the method comprises (a) introducing into a population of cells from (or of) the organism a protein-RNA complex, the protein-RNA complex comprising: (i) a Clustered Regularly Interspaced Short Palindromic Repeats (CRISPR) comprising one or more (e.g., 1, 2, 3, 4, 5, 6, 7, 8, 9, 10, 11, 12, 13, 14, 15 or more) repeat sequence(s) and one or more (e.g., 1, 2, 3, 4, 5, 6, 7, 8, 9, 10, 11, 12, 13, 14, 15 or more) spacer sequence(s), wherein each spacer sequence is linked at its 5′ end and at its 3′ end to a repeat sequence, and the spacer sequence is complementary to a target sequence (protospacer) in a target nucleic acid of (at least a portion of the population of cells of) the organism (wherein the target sequence is not present in the variant cell), wherein the target sequence is located immediately adjacent (3′) to a protospacer adjacent motif (PAM); (ii) a Type I-B CRISPR associated complex for antiviral defense complex (Cascade complex) comprising (i) a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1, a Cas8 polypeptide having at least 80% sequence identity to the amino acid sequence of SEQ ID NO:2, a Cas7 polypeptide having at least 80% sequence identity to the amino acid sequence of SEQ ID NO:3, a Cas5 polypeptide having at least 80% sequence identity to the amino acid sequence of SEQ ID NO: 4, and a Cas3 polypeptide having at least 80% sequence identity to the amino acid sequence of SEQ ID NO:5; or (ii) a Cas6 polypeptide having at least 80% sequence identity to the amino acid sequence of SEQ ID NO:19, a Cas8 polypeptide having at least 80% sequence identity to the amino acid sequence of SEQ ID NO:20, a Cas7 polypeptide having at least 80% sequence identity to the amino acid sequence of SEQ ID NO:21, a Cas5 polypeptide having at least 80% sequence identity to the amino acid sequence of SEQ ID NO: 22, and a Cas3 polypeptide having at least 80% sequence identity to the amino acid sequence of SEQ ID NO:23, wherein the recombinant nucleic acid construct comprising a CRISPR and the recombinant nucleic acid construct encoding a Cascade complex each comprise a polynucleotide encoding a polypeptide conferring resistance to a selection agent, thereby [killing transformed cells comprising the target sequence and] producing a subpopulation of cells of the population of cells; and (b) selecting from the subpopulation of cells produced in (a) one or more cells that are resistant to the selection agent, thereby selecting one or more variant cells that do not comprise the target sequence and are not killed.

In some embodiments, a method of killing one or more cells in a population of bacterial and/or archaeal cells is provided, the method comprising introducing into the one or more cells of the population of bacterial and/or archaeal cells: (a) a recombinant nucleic acid construct comprising a Clustered Regularly Interspaced Short Palindromic Repeats (CRISPR) comprising one or more (e.g., 1, 2, 3, 4, 5, 6, 7, 8, 9, 10, 11, 12, 13, 14, 15 or more) repeat sequence(s) and one or more (e.g., 1, 2, 3, 4, 5, 6, 7, 8, 9, 10, 11, 12, 13, 14, 15 or more) spacer nucleotide sequence(s), wherein each of the one or more spacer sequences comprises a 3′ end and a 5′ end and is linked at its 5′ end and at its 3′ end to a repeat sequence, and each of the one or more spacer sequences is complementary to a target sequence (protospacer) in the genome of the bacterial and/or archaeal cells of the population, and wherein the target sequence is a genomic sequence that is present in (e.g., conserved among) the one or more cells within the population of bacterial and/or archaeal cells and the target sequence is located immediately adjacent (3′) to a protospacer adjacent motif (PAM); and (b) a recombinant nucleic acid construct encoding a Type I-B CRISPR associated complex for antiviral defense complex (Cascade complex) comprising: (i) a Cas6 polypeptide having at least 80% sequence identity to the amino acid sequence of SEQ ID NO:1, a Cas8 polypeptide having at least 80% sequence identity to the amino acid sequence of SEQ ID NO: 2, a Cas7 polypeptide having at least 80% sequence identity to the amino acid sequence of SEQ ID NO:3, a Cas5 polypeptide having at least 80% sequence identity to the amino acid sequence of SEQ ID NO:4, or a Cas3 polypeptide having at least 80% sequence identity to the amino acid sequence of SEQ ID NO:5; or (ii) a Cas6 polypeptide having at least 80% sequence identity to the amino acid sequence of SEQ ID NO:19, a Cas8 polypeptide having at least 80% sequence identity to the amino acid sequence of SEQ ID NO:20, a Cas7 polypeptide having at least 80% sequence identity to the amino acid sequence of SEQ ID NO: 21, a Cas5 polypeptide having at least 80% sequence identity to the amino acid sequence of SEQ ID NO:22, or a Cas3 polypeptide having at least 80% sequence identity to the amino acid sequence of SEQ ID NO:23, thereby killing one or more cells in the population of bacterial and/or archaeal cells that comprise the target sequence in their genome.

In some embodiments, the present invention provides a method of killing one or more cells in a population of bacterial and/or archaeal cells, the method comprising introducing into the one or more cells of the population of bacterial and/or archaeal cells a protein-RNA complex, the protein-RNA complex comprising: (a) a Clustered Regularly Interspaced Short Palindromic Repeats (CRISPR) comprising one or more (e.g., 1, 2, 3, 4, 5, 6, 7, 8, 9, 10 or more) repeat sequence(s) and one or more (e.g., 1, 2, 3, 4, 5, 6, 7, 8, 9, 10 or more) spacer nucleotide sequence(s), wherein each of the one or more spacer sequences comprises a 3′ end and a 5′ end and is linked at its 5′ end and at its 3′ end to a repeat sequence, and each of the one or more spacer sequences is complementary to a target sequence (protospacer) in the genome of the bacterial and/or archaeal cells of the population, wherein the target sequence is a genomic sequence that is conserved among the one or more cells within the population of bacterial and/or archaeal cells and the target sequence is located immediately adjacent (3′) to a protospacer adjacent motif (PAM); and (b) a Type I-B CRISPR associated complex for antiviral defense complex (Cascade complex) comprising (i) a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1, a Cas8 polypeptide having at least 80% sequence identity to the amino acid sequence of SEQ ID NO:2, a Cas7 polypeptide having at least 80% sequence identity to the amino acid sequence of SEQ ID NO: 3, a Cas5 polypeptide having at least 80% sequence identity to the amino acid sequence of SEQ ID NO:4, and a Cas3 polypeptide having at least 80% sequence identity to the amino acid sequence of SEQ ID NO:5; or (ii) a Cas6 polypeptide having at least 80% sequence identity to the amino acid sequence of SEQ ID NO:19, a Cas8 polypeptide having at least 80% sequence identity to the amino acid sequence of SEQ ID NO:20, a Cas7 polypeptide having at least 80% sequence identity to the amino acid sequence of SEQ ID NO:21, a Cas5 polypeptide having at least 80% sequence identity to the amino acid sequence of SEQ ID NO: 22, and a Cas3 polypeptide having at least 80% sequence identity to the amino acid sequence of SEQ ID NO:23, thereby killing one or more cells in the population of bacterial and/or archaeal cells that comprise the target sequence in their genome.

In some embodiments, a population of cells (e.g., for methods of screening, selecting, or killing) may be obtained from a single multicellular organism or may be obtained from a population of different individuals of an organism.

A bacterial cell for use with this invention may be a single cell or a cell within a population of bacterial cells of the same species or strain or may be a cell within a population comprising a mixture of two or more bacterial species or strains. In some embodiments, the methods of this invention (e.g., enhancing resistance to one or more bacteriophage species or strains) may be carried out on a portion of a population of bacterial cells. As used herein, “at least a portion of the population of cells” means at least one cell of a population of two or more cells (e.g., 2, 3, 4, 5, 6, 7, 8, 9, 10, or more cells, e.g., 10², 10³, 10⁴, 10⁵, 10⁶, 10⁷, 10⁸, 10⁹, 10¹⁰ or more cells). In some embodiments, the bacterial cell is a cell of a commensal bacterial species or strain, optionally the bacterial cell is a cell of a commensal Clostridium spp. or strain. In some embodiments, the bacterial cell may be a Clostridium spp. cell or a Erysipelatoclostridium ramosum cell.

Further provided herein is a method of killing one or more cells in a population of bacterial and/or archaeal cells that comprise an endogenous Type I-B Clustered Regularly Interspaced Short Palindromic Repeats (CRISPR)-Cas system, the method comprising introducing into the one or more cells of the population of bacterial and/or archaeal cells a recombinant nucleic acid construct comprising a CRISPR (that is functional with at least the endogenous CRISPR-Cas system of the one or more bacterial cells in the population) comprising one or more (e.g., 1, 2, 3, 4, 5, 6, 7, 8, 9, 10, 11, 12, 13, 14, 15 or more) repeat sequence(s) and one or more (e.g., 1, 2, 3, 4, 5, 6, 7, 8, 9, 10, 11, 12, 13, 14, 15 or more) spacer nucleotide sequence(s), wherein each of the one or more spacer sequences comprises a 3′ end and a 5′ end and is linked at its 5′ end and at its 3′ end to a repeat sequence, and each of the one or more spacer sequences is complementary to a target sequence (protospacer) in a target DNA in the one or more bacterial and/or archaeal cells of the population, wherein the target sequence is present in (e.g., conserved among) the one or more cells within the population of bacterial and/or archaeal cells and the target sequence is located immediately adjacent (3′) to a protospacer adjacent motif (PAM), thereby killing the one or more cells within the population of bacterial and/or archaeal cells that comprise the target sequence in their genome. In some embodiments, a target sequence may be an essential and/or non-expendable, or a non-essential and/or expendable, genomic sequence located on a chromosome. In some embodiments, a target sequence may be an essential and/or non-expendable genomic sequence located on a chromosome.

Thus, for example, transformation of bacterial or archaeal genome-targeting CRISPRs can be used to selectively kill bacterial or archaeal cells on a sequence-specific basis to subtract genetically distinct subpopulations, thereby enriching bacterial populations lacking the target sequence. This distinction can occur on the basis of the heterogeneous distribution of orthogonal CRISPR-Cas systems within genetically similar populations. Thus, in some embodiments, a CRISPR that is introduced into a population of cells can be compatible (i.e., functional) with a CRISPR-Cas system in the one or more bacterial or archaeal cells to be killed but is not compatible (i.e., not functional) with the CRISPR Cas system of at least one or more bacterial or archaeal cells in the population. For instance, Escherichia coli and Klebsiella pneumoniae can exhibit either Type I-E or Type I-F CRISPR-Cas systems; Clostridium difficile exhibits Type I-B systems, and different strains of S. thermophilus exhibit both Type II-A and Type I-E systems or only Type II-A systems. Depending on the specific CRISPR transformed into a mixed population of bacteria, the CRISPR can specifically target that subset of the population based on its functional compatibility with its cognate system. This can be applied to diverse species containing endogenous CRISPR-Cas systems such as, but not limited to: Pseudomonas spp. (such as: P. aeruginosa), Escherichia spp. (such as: E. coli), Enterobacter spp. (such as: E. cloacae), Staphylococcus spp. (such as: S. aureus), Enterococcus spp. (such as: E. faecalis, E. faecium), Streptomyces spp. (such as: S. somaliensis), Streptococcus spp. (such as: S. pyogenes), Vibrio spp. (such as: V. cholerae), Yersinia spp. (such as: Y. pestis), Francisella spp. (such as: F. tularensis, F. novicida), Bacillus spp. (such as: B. anthracis, B. cereus), Lactobacillus spp. (such as: L. casei, L. reuteri, L. acidophilus, L. rhamnosus), Burkholderia spp. (such as: B. mallei, B. pseudomallei), Klebsiella spp. (such as: K. pneumoniae), Shigella spp. (such as: S. dysenteriae, S. sonnei), Salmonella spp. (such as: S. enterica), Borrelia spp. (such as: B. burgdorferi), Neisseria spp. (such as: N. meningitidis), Fusobacterium spp. (such as: F. nucleatum), Helicobacter spp. (such as: H. pylori), Chlamydia spp. (such as: C. trachomatis), Bacteroides spp. (such as: B. fragilis), Bartonella spp. (such as: B. quintana), Bordetella spp. (such as: B. pertussis), Brucella spp. (such as: B. abortus), Campylobacter spp. (such as: C. jejuni), Clostridium spp. (such as: C. difficile), Bifidobacterium spp. (such as: B. infantis), Haemophilus spp. (such as: H. influenzae), Listeria spp. (such as: L. monocytogenes), Legionella spp. (such as: L. pneumophila), Mycobacterium spp. (such as: M. tuberculosis), Mycoplasma spp. (such as: M. pneumoniae), Rickettsia spp. (such as: R. rickettsii), Acinetobacter spp. (such as: A. calcoaceticus, A. baumanii), Ruminococcus spp. (such as: R. albus), Propionibacterium spp. (such as: P. freudenreichii), Corynebacterium spp. (such as: C. diphtheriae), Propionibacterium spp. (such as: P. acnes), Brevibacterium spp. (such as: B. iodinum), Micrococcus spp. (such as: M. luteus), and/or Prevotella spp. (such as: P. histicola).

CRISPR targeting can remove specific bacterial subsets on the basis of the distinct genetic content in mixed populations. CRISPR-targeting spacers can be tuned to various levels of bacterial relatedness by targeting conserved or divergent genetic sequences. Thus, in some embodiments, the bacterial and/or archaeal cells in a population may comprise the same CRISPR-Cas system and the introduced CRISPR thus may be functional in the bacterial population as a whole but the genetic content of the different strains or species that make up the bacterial and/or archaeal population may be sufficiently distinct such that the target region for the introduced CRISPR is found only in the one or more bacterial species of the population that is to be killed. This can be applied to diverse species containing endogenous CRISPR-Cas systems such as, but not limited to: Pseudomonas spp. (such as: P. aeruginosa), Escherichia spp. (such as: E. coli), Enterobacter spp. (such as: E. cloacae), Staphylococcus spp. (such as: S. aureus), Enterococcus spp. (such as: E. faecalis, E. faecium), Streptomyces spp. (such as: S. somaliensis), Streptococcus spp. (such as: S. pyogenes), Vibrio spp. (such as: V. cholerae), Yersinia spp. (such as: Y. pestis), Francisella spp. (such as: F. tularensis, F. novicida), Bacillus spp. (such as: B. anthracis, B. cereus), Lactobacillus spp. (such as: L. casei, L. reuteri, L. acidophilus, L. rhamnosus), Burkholderia spp. (such as: B. mallei, B. pseudomallei), Klebsiella spp. (such as: K. pneumoniae), Shigella spp. (such as: S. dysenteriae, S. sonnei), Salmonella spp. (such as: S. enterica), Borrelia spp. (such as: B. burgdorferi), Neisseria spp. (such as: N. meningitidis), Fusobacterium spp. (such as: F. nucleatum), Helicobacter spp. (such as: H. pylori), Chlamydia spp. (such as: C. trachomatis), Bacteroides spp. (such as: B. fragilis), Bartonella spp. (such as: B. quintana), Bordetella spp. (such as: B. pertussis), Brucella spp. (such as: B. abortus), Campylobacter spp. (such as: C. jejuni), Clostridium spp. (such as: C. difficile), Bifidobacterium spp. (such as: B. infantis), Haemophilus spp. (such as: H. influenzae), Listeria spp. (such as: L. monocytogenes), Legionella spp. (such as: L. pneumophila), Mycobacterium spp. (such as: M. tuberculosis), Mycoplasma spp. (such as: M. pneumoniae), Rickettsia spp. (such as: R. rickettsii), Acinetobacter spp. (such as: A. calcoaceticus, A. baumanii), Ruminococcus spp. (such as: R. albus), Propionibacterium spp. (such as: P. freudenreichii), Corynebacterium spp. (such as: C. diphtheriae), Propionibacterium spp. (such as: P. acnes), Brevibacterium spp. (such as: B. iodinum), Micrococcus spp. (such as: M. luteus), and/or Prevotella spp. (such as: P. histicola).

The extent of killing within a population using the methods of this invention may be affected by the amenability of the particular population to transformation in addition to whether the target region is comprised in a non-essential gene, an essential gene or an expendable island. The extent of killing in a population of bacterial or archaeal cells may vary, for example, by organism, by genus and species. Accordingly, as used herein “killing” means eliminating at least about 1 to about 3 logs (e.g., 1, 1.25, 1.5, 1.75, 2, 2.25, 2.5, 2.75, 3 or any range or value therein) or more of the cells in a population (10% survival or less (e.g., about 0 to 10%, about 1% to 10%, about 1% to 8%, about 1% to 5%, about 5% to about 10% and the like, and any range or value therein) (e.g., about 0%, 1%, 2%, 3%, 4%, 5%, 6%, 7%, 8%, 9%, 10%, and any range or value therein)). One log of killing (e.g., about 90% killing) may be a small reduction in the population but may suffice for the purposes of the invention of reducing a population. Two to three logs of killing provide a significant reduction of the population; and more than 3 logs of killing indicates that the population has been substantially eradicated.

In some embodiments, PAM sequences useful with the Type I-B CRISPR-Cas systems of this invention are located immediately adjacent to and 5′ of the target sequence (protospacer) and include, but are not limited to, the nucleotide sequence of 5′-TTTA-3′ and/or the nucleotide sequences of 5′-ATC-3′, 5′-ATC-3′ or 5′-ATCN-3′ (N=A, T, C, or G, e.g., 5′-ATCG-3′, 5′-ATCA-3′, 5′-ATCC-3′, 5′-ATCT-3′).

A CRISPR useful with the methods of the invention may comprise one or more (e.g., 1, 2, 3, 4, 5, 6, 7, 8, 9, 10 or more) or two or more (e.g., 2, 3, 4, 5, 6, 7, 8, 9, 10 or more) repeat sequences and one or more (e.g., 1, 2, 3, 4, 5, 6, 7, 8, 9, 10 or more) spacer sequence(s), wherein each spacer sequence and each repeat sequence have a 5′ end and a 3′ end and each spacer sequence is linked at its 5′ end, and optionally at its 3′ end, to a repeat sequence (or portion thereof), and the spacer sequence is complementary to a target sequence (protospacer) in a target DNA of a target organism that is located immediately adjacent (3′) to a protospacer adjacent motif (PAM). In some embodiments, a CRISPR of the invention comprising at least one spacer sequence and at least two repeat sequences (or portion thereof) flanking the spacer, may be expressed as a premature CRISPR RNA (pre-crRNA) that will be processed internally in the cell to constitute the final mature CRISPR RNA (crRNA). In some embodiments, a CRISPR RNA (crRNA) of the present invention may comprise a processed crRNA comprising at least one repeat sequence (or portion thereof) and a spacer sequence, wherein the at least one repeat sequence (or portion thereof) is linked to the 5′ end of the spacer sequence.

In some embodiments, a repeat sequence (i.e., CRISPR repeat sequence) as used herein may comprise any known repeat sequence of a wild-type Clostridium CRISPR Type I-B locus (e.g., E. ramosum or Clostridium spp. 1141A1FAA). In some embodiments, a repeat sequence useful with the invention may include a synthetic repeat sequence having a different nucleotide sequence than those known in the art for Clostridium but sharing similar structure to that of wild-type Clostridium repeat sequences of a hairpin structure with a loop region. Thus, in some embodiments, a repeat sequence may be identical to (i.e., having 100% identity) or substantially identical (e.g., having 80% to 99% identity (e.g., about 80, 81, 82, 83, 84, 85, 86, 87, 88, 89, 90, 91, 92, 93, 94, 95, 96, 97, 98, 99% identity)) to a repeat sequence from a wild-type Clostridium CRISPR Type I-B locus.

The length of a CRISPR repeat sequence useful with the recombinant nucleic acid constructs and methods of the invention may be the full length of a Clostridium spp. or Erysipelatoclostridium spp. (e.g., E. ramosum or C. spp. 1141A1FAA) repeat sequence (i.e., about 27 to 32 nucleotides, e.g., 27, 28, 29, 30, 31, or 32) (see, e.g., SEQ ID NOs: 17, 18 or 35-43). In some embodiments, a repeat sequence may comprise a portion of a wild type Clostridium or Erysipelatoclostridium repeat nucleotide sequence, the portion being reduced in length by as much as 7 to 8 nucleotides (e.g., 1, 2, 3, 4, 5, 6, 7, or 8 nucleotides) from the 3′ end as compared to a wild type Clostridium or Erysipelatoclostridium repeat (e.g., comprising about 19 to 31 contiguous nucleotides from the 5′ end of a wild type Clostridium or Erysipelatoclostridium CRISPR Type I-B locus repeat sequence; e.g., about 19, 20, 20, 21, 22, 23, 24, 25, 26, 27, 28, 29, 30 or 31 contiguous nucleotides from the 5′ end, or any range or value therein). In some embodiments, a repeat sequence useful with this invention may comprise, consist essentially of or consist of at least 19 consecutive nucleotides (e.g., about 19, 20, 21, 22, 23, 24, 25, 26, 27, 28, 29, 30, 31, 32 or 33 consecutive nucleotides) having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the nucleotide sequences of SEQ ID NOs: 17, 18 or 35-43, optionally about 19, 20, 21, 22, 23, 24, or 25 consecutive nucleotides to about 26, 27, 28 29, 30, or 31 consecutive nucleotides, about 21, 22, 23, 24, or 25 consecutive nucleotides to about 26, 27, 28, 29, 30, 31, 32 or 33, or about 27 to about 32 consecutive nucleotides of a repeat sequence.

Thus, in some embodiments, a repeat sequence may comprise, consist essentially of, or consist of any of the nucleotide sequences of SEQ ID NO: 17, SEQ ID NO:18, SEQ ID NO: 35, SEQ ID NO:36, SEQ ID NO:37, SEQ ID NO:38, SEQ ID NO:39, SEQ ID NO: 40, SEQ ID NO:41, SEQ ID NO:42, and/or SEQ ID NO:43 or any combination thereof. In some embodiments, a repeat sequence may comprise, consist essentially of, or consist of a portion of contiguous nucleotides as described herein, of any of the nucleotide sequences of SEQ ID NOs: 17, 18, 35, 36, 37, 38, 39, 40, 41, 42 or 43, or any combination thereof.

In some embodiments, when two or more (e.g., 2, 3, 4, 5, 6, 7, 8, 9, 10, 11, 12, 13, 14, 15 or more) repeat sequences are present in a CRISPR may comprise the same repeat sequence, may comprise different repeat sequences, or any combination thereof. In some embodiments, each of the two or more repeat sequences in a single CRISPR may comprise, consist essentially of, or consist of the same repeat sequence.

A CRISPR useful with the invention may comprise one spacer sequence or more than one spacer sequence, wherein each spacer sequence is flanked by at least a repeat sequence on the 5′ end of the spacer (3′ end of the repeat linked to the 5′ end of the spacer, e.g., repeat-spacer). In some embodiments, a spacer sequence may be linked on the 5′ end and the 3′ end to a repeat sequence (e.g., repeat-spacer-repeat). When more than one spacer sequence is present in a CRISPR of the invention, each spacer sequence is separated from the next spacer sequence by a repeat sequence. Thus, each spacer sequence is linked at the 3′ end and at the 5′ end to a repeat sequence. The repeat sequence that is linked to each end of the one or more spacers may be the same repeat sequence or it may be a different repeat sequence, or any combination thereof.

In some embodiments, the one or more spacer sequences of the present invention may be about 25 nucleotides to about 60 nucleotides in length (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length, and any value or range therein). In some embodiments, a spacer sequence may be a length of about 25 to about 45 nucleotides (e.g., about 25, 26, 27, 28, 29, 30, 31, 32, 33, 34, 35, 36, 37, 38, 39, 40, 41, 42, 43, 44, or 45 nucleotides in length, and any value or range therein) or about 30 to about 40 nucleotides (e.g., about 30, 31, 32, 33, 34, 35, 36, 37, 38, 39, or 40 nucleotides in length, and any value or range therein). In some embodiments, a spacer sequence may comprise, consist essentially of, or consist of a length of about 34, 35, 36 or 37 nucleotides in length. In some embodiments, a spacer sequence may comprise, consist essentially of, or consist of a length of about 34 nucleotides in length. In some embodiments, a spacer sequence may comprise, consist essentially of, or consist of a length of about 35 nucleotides in length. In some embodiments, a spacer sequence may comprise, consist essentially of, or consist of a length of about 36 nucleotides in length. In some embodiments, a spacer sequence may comprise, consist essentially of, or consist of a length of about 37 nucleotides in length.

In some embodiments, a spacer sequence useful with the methods of this invention may be fully complementary to a target sequence (e.g., 100% complementary to a target sequence across its full length). In some embodiments, a spacer sequence may be substantially complementary (e.g., at least about 80% complementary (e.g., about 80%, 81%, 82%, 83%, 84%, 85%, 86%, 87%, 88%, 89%, 90%, 91%, 92%, 93%, 94%, 95%, 96%, 97%, 98%, 98.5%, 99%, 99.5%, or more complementary)) to a target sequence from a target genome. Thus, in some embodiments, a spacer sequence may have one, two, three, four, five or more mismatches that may be contiguous or noncontiguous as compared to a target sequence from a target genome. In some embodiments, a spacer sequence may be about 80% to 100% (e.g., about 80%, 81%, 82%, 83%, 84%, 85%, 86%, 87%, 88%, 89%, 90%, 91%, 92%, 93%, 94%, 95%, 96%, 97%, 98%, 99%, or 100%) complementary to a target sequence from a target genome. In some embodiments, a spacer sequence may be about 85% to 100% (e.g., about 85%, 86%, 87%, 88%, 89%, 90%, 91%, 92%, 93%, 94%, 95%, 96%, 97%, 98%, 99%, or 100%) complementary to a target sequence from a target genome. In some embodiments, a spacer sequence may be about 90% to 100% (e.g., about 90%, 91%, 92%, 93%, 94%, 95%, 96%, 97%, 98%, 99%, or 100%)) or about 95% to 100% (e.g., about 95%, 96%, 97%, 98%, 98.5%, 99%, 99.5% or 100%) complementary to a target sequence from a target genome.

In some embodiments, the 5′ region of a spacer sequence may be fully complementary to a target sequence while the 3′ region of the spacer sequence may be substantially complementary to the target sequence. Accordingly, in some embodiments, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) may be about 100% complementary to a target sequence, while the remainder of the spacer sequence may be about 80% or more complementary to the target sequence.

In some embodiments, at least the first eight contiguous nucleotides at the 5′ end of a spacer sequence of the invention are fully complementary to the portion of the target sequence adjacent to the PAM (termed a “seed sequence”). Thus, in some embodiments, the seed sequence may comprise the first 8 nucleotides of the 5′ end of each of one or more spacer sequence(s), which first 8 nucleotides are fully complementary (100%) to the target sequence, and the remaining portion of the one or more spacer sequence(s) (3′ to the seed sequence) may be at least about 80% complementarity (e.g., about 80, 81, 82, 83, 84, 85, 86, 87, 88, 89, 90, 91, 92, 93, 94, 95, 96, 97, 98, 99, or 100% complementarity) to the target sequence. Thus, for example, a spacer sequence having a length of 28 nucleotides may comprise a seed sequence of eight contiguous nucleotides located at the 5′ end of the spacer sequence, which is 100% complementary to the target sequence, while the remaining 20 nucleotides may be about 80% to about 100% complimentary to the target sequence (e.g., 0 to 4 non-complementary nucleotides out of the remaining 20 in the spacer sequence). As another example, a spacer sequence having a length of 33 nucleotides may comprise a seed sequence of eight nucleotides from the 5′ end, which is 100% complementary to the target sequence, while the remaining 25 nucleotides may be at least about 80% (e.g., 0 to 5 non-complementary nucleotides out of the remaining 25 nucleotides in the spacer sequence) or a spacer sequence having a length of 37 nucleotides may comprise a seed sequence of eight nucleotides from the 5′ end, which is 100% complementary to the target sequence, while the remaining 29 nucleotides may be at least about 80% (e.g., 0 to 5 non-complementary nucleotides out of the remaining 29 nucleotides in the spacer sequence).

A CRISPR of the invention comprising more than one spacer sequence may be designed to target one or more than one target sequence (protospacer) in an organism or cell thereof. Thus, in some embodiments, when a recombinant nucleic acid construct of the invention comprises a CRISPR that comprises at least two spacer sequences, the at least two spacer sequences may be complementary to two or more different target sequences in the organism or cell thereof. In some embodiments, when a recombinant nucleic acid construct of the invention comprises a CRISPR that comprises at least two spacer sequences, the at least two spacer sequences may be complementary to the same target sequence. In some embodiments, a CRISPR comprising at least two spacer sequences, the at least two spacer sequences may be complementary to different portions of one gene.

In some embodiments, more than one CRISPR may be introduced into a cell or a cell free system using various combinations of the constructs as described herein. In some embodiments, a recombinant nucleic acid construct comprising one CRISPR may be introduced into a cell or cell free system or a recombinant nucleic acid construct comprising more than one CRISPR may be introduced into a cell or cell free system. In some embodiments, more than one recombinant nucleic acid construct each comprising one CRISPR or more than one CRISPR may be introduced into a cell or cell free system.

In some embodiments, a recombinant nucleic acid construct comprising a CRISPR, a recombinant nucleic acid construct encoding a Cascade complex, and optionally a recombinant nucleic acid construct encoding a Cas3, may be introduced into the target organism or cell of the target organism simultaneously, separately and/or sequentially. Thus, in some embodiments, a recombinant nucleic acid construct comprising a CRISPR and/or the recombinant nucleic acid construct encoding a Cascade complex may be comprised in a single vector and/or expression cassette or may be comprised in two or three separate vectors and/or expression cassettes, optionally wherein the vector may be, for example, a recombinant plasmid, bacteriophage, transposon, phagemid, or retrovirus. In some embodiments, the recombinant nucleic acid construct comprising a CRISPR and the recombinant nucleic acid construct encoding a Cascade complex are comprised in the same vector and therefore, introduced together.

When introduced into a target organism, a cell of a target organism or into a cell free system, a recombinant nucleic acid construct comprising a CRISPR and a recombinant nucleic acid construct encoding a Cascade complex (with or without a Cas3 polypeptide) may be introduced into the target organism, the cell of the target organism or the cell free system simultaneously, separately and/or sequentially, in any order. In some embodiments, a recombinant nucleic acid construct comprising a CRISPR and a recombinant nucleic acid construct encoding a Cascade complex may be introduced simultaneously on the same or on separate expression cassettes and/or vectors. In some embodiments, the recombinant nucleic acid construct comprising a CRISPR and the recombinant nucleic acid construct encoding a Cascade complex are introduced simultaneously on a single expression cassette and/or vector. In some embodiments, when co-opting an endogenous CRISPR-Cas Type I-B system of a bacterium and/or archaeon (for example, when a bacterium or archaeon has an endogenous CRISPR-Cas system that is functional with the CRISPRs of the present invention) only a recombinant nucleic acid construct comprising a CRISPR of the invention is introduced.

In some embodiments, when a recombinant nucleic acid construct comprising a CRISPR and a recombinant nucleic acid construct encoding a Cascade complex (with or without a Cas3 polypeptide) polypeptide are introduced into a cell, they may be comprised in a single expression cassette and/or vector in any order. In some embodiments, when a recombinant nucleic acid construct comprising a CRISPR and a recombinant nucleic acid construct encoding a Cascade complex are introduced into a cell, they may be comprised in two or three separate vectors and/or expression cassettes in any order. When more than one expression cassette and/or vector is used to introduce the constructs of the invention, each may encode different selection agents/markers (e.g., may encode nucleic acids conferring resistance to different antibiotics) so that the transformed cell maintains each expression cassette/vector that is introduced.

Non-limiting examples of vectors useful with this invention include plasmids, bacteriophage, transposons, phagemids, or retroviruses.

TABLE 1
Example combinations of Cascade polypeptides and nucleotide sequences,
repeat sequences and PAM sequences of the invention.

| | Cascade proteins/polynucleotides | Repeat | |
|---|---|---|---|
| Organism | SEQ ID NOs | SEQ ID NOs | PAM |
| E. ramosum | SEQ ID NO: 1/SEQ ID NO: 9 (Cas6) | SEQ ID NO: 17 | 5′-ATC-3′ |
| DSM1402 | SEQ ID NO: 2/SEQ ID NO: 10 (Cas8) | SEQ ID NO: 18 | 5′-ATCN*-3′ |
| | SEQ ID NO: 3/SEQ ID NO: 11 (Cas7) | | 5′-ATCG-3′ |
| | SEQ ID NO: 4/SEQ ID NO: 12 (cas5) | | 5′-ATCA-3′ |
| | SEQ ID NO: 5/SEQ ID NO: 13(Cas3) | | |
| Clostridium spp. | SEQ ID NO: 19/SEQ ID NO: 27 (Cas6) | SEQ ID NOs: | 5′-TTTA-3′ |
| 1141A1FAA | SEQ ID NO: 20/SEQ ID NO: 28 (Cas8) | 35, 36, 37, | |
| | SEQ ID NO: 21/SEQ ID NO: 29 (Cas7) | 38, 39, 40, | |
| | SEQ ID NO: 22/SEQ ID NO: 30 (Cas5) | 41, 42, 43 | |
| | SEQ ID NO: 23/SEQ ID NO: 31(Cas3) | | |

*N = A, T, C, G

As described herein, the constructs of the invention may optionally comprise regulatory elements, including, but not limited to, promoters and terminators. Promoters useful with the methods of the invention are as described herein, and include, but are not limited to the nucleotide sequences of SEQ ID NOs: 44-52, and any combination thereof. In some embodiments, when more than one construct is introduced, promoters useful with the constructs may be any combination of heterologous and/or endogenous promoters.

Thus, in some embodiments, a recombinant nucleic acid construct comprising a CRISPR and a recombinant nucleic acid construct encoding a Cascade complex may be operably linked to a single promoter, in any order or in any combination thereof, or they may each be operably linked to independent (e.g., separate) promoters. In some embodiments, when a recombinant nucleic acid construct comprising a CRISPR and a recombinant nucleic acid construct encoding a Cascade complex are present in the same expression cassette and/or vector, they may be operably linked to the same promoter. In some embodiments, when a recombinant nucleic acid construct comprising a CRISPR and a recombinant nucleic acid construct encoding a Cascade complex are present in the same expression cassette or vector, the recombinant nucleic acid construct encoding a Cascade complex and the recombinant nucleic acid construct encoding a CRISPR may be operably linked to separate promoters that may be the same or different. Promoters useful with the methods of the invention are as described herein, and include, but are not limited to the nucleotide sequences of SEQ ID NOs: 44-52, in any combination.

In some embodiments, a recombinant nucleic acid construct comprising a CRISPR may be operably linked to a terminator and a recombinant nucleic acid construct encoding a Cascade complex may be optionally operably linked to a terminator. In some embodiments, a recombinant nucleic acid construct comprising a CRISPR, a recombinant nucleic acid construct encoding a Cascade complex may each be operably linked to a single terminator, in any order or in any combination thereof, or they may each be operably linked to independent (e.g., separate) terminators. In some embodiments, when a recombinant nucleic acid construct comprising a CRISPR and a recombinant nucleic acid construct encoding a Cascade complex are present in the same expression cassette or vector, they may be operably linked to the same terminator. In some embodiments, when a recombinant nucleic acid construct comprising a CRISPR and a recombinant nucleic acid construct encoding a Cascade complex are present in the same expression cassette and/or vector, only the recombinant nucleic acid construct encoding a CRISPR is operably linked to a terminator sequence. Terminator sequences useful with the methods of the invention are as described herein. In some embodiments, a terminator sequence useful with the invention may include, but is not limited to, the nucleotide sequence of any one of SEQ ID NOs: 53-61, and/or any combination thereof.

Notably, the recombinant nucleic acid constructs, protein-RNA complexes and their methods of use as described herein are advantageous over other known CRISPR systems in that their activity (as measured by repression reaching up to 98%) is quite high. In addition, the PAM (TTTA, ATCN) which are quite distinct from and complementary to known systems that are GC rich (for example, the TTTA PAM enables targeting of AAAT complementary sequences on the other strand, with noteworthy AT bias highly distinct from and complementary to GC-rich PAMs previously reported). Another advantage is the long spacer (e.g., about 34-37 nt) which provides expanded opportunities for specificity. The present invention further provides sequence and structural diversity from other known Type I systems (see, e.g., the widely used E coli system), with different CRISPR repeat sequences and longer 5′ handle and 3′ hairpins, which provides opportunities for concurrent use of two (or more) orthogonal systems that provide multiplexed opportunities to perform multiple reactions that are different all at the same time (e.g., up-regulation, and down-regulation and/or genome editing).

“Introducing,” “introduce,” “introduced” (and grammatical variations thereof) in the context of a polynucleotide of interest and a cell of an organism means presenting the polynucleotide of interest to the host organism or cell of said organism (e.g., host cell) in such a manner that the nucleotide sequence gains access to the interior of a cell and includes such terms as transformation,” “transfection,” and/or “transduction.” Transformation may be electrical (electroporation and electrotransformation), or chemical (with a chemical compound, and/or though modification of the pH and/or temperature in the growth environment. Where more than one nucleotide sequence is to be introduced these nucleotide sequences can be assembled as part of a single polynucleotide or nucleic acid construct, or as separate polynucleotide or nucleic acid constructs, and can be located on the same or different expression constructs or transformation vectors. Accordingly, these polynucleotides can be introduced into cells in a single transformation event, in separate transformation events, or, for example, they can be incorporated into an organism by conventional breeding or growth protocols. Thus, in some aspects of the present invention one or more recombinant nucleic acid constructs of this invention may be introduced into a host organism or a cell of said host organism.

“Introducing,” “introduce,” “introduced” (and grammatical variations thereof) in the context of a protein-RNA complex of the invention and a cell of an organism means presenting the polynucleotide of interest to the host organism or cell of said organism and includes such terms as transformation,” “transfection,” and/or “transduction.” Thus, in some embodiments, the terms “transformation,” “transfection,” and “transduction” as used herein may also refer to the introduction of a protein-RNA complex of the invention into a cell.

The terms “transformation,” “transfection,” and “transduction” as used herein refer to the introduction of a heterologous nucleic acid into a cell. Such introduction into a cell may be stable or transient. Thus, in some embodiments, a host cell or host organism is stably transformed with a nucleic acid construct of the invention. In other embodiments, a host cell or host organism is transiently transformed with a recombinant nucleic acid construct of the invention.

As used herein, the term “stably introduced” means that the introduced polynucleotide is stably incorporated into the genome of the cell, and thus the cell is stably transformed with the polynucleotide. When a nucleic acid construct is stably transformed and therefore integrated into a cell, the integrated nucleic acid construct is capable of being inherited by the progeny thereof, more particularly, by the progeny of multiple successive generations. In some embodiments, the term “stably introduced” means that an introduced protein-RNA complex of the invention is stably maintained in the cell into which it is introduced.

“Transient transformation” in the context of a polynucleotide or a protein-RNA complex means that a polynucleotide or the protein-RNA complex is introduced into the cell and does not integrate into the genome of the cell or is not otherwise maintained by the cell.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant, a mammal, an insect, an archaea, a bacterium, and the like). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, the nucleotide sequences, constructs, expression cassettes may be expressed transiently and/or they may be stably incorporated into the genome of the host organism. In some embodiments, when transient transformation is desired, the loss of the plasmids and the recombinant nucleic acids comprised therein may achieved by removal of selective pressure for plasmid maintenance.

A recombinant nucleic acid construct of the invention or a protein-RNA complex of the invention may be introduced into a cell by any method known to those of skill in the art. Exemplary methods of transformation or transfection include biological methods using viruses and bacteria (e.g., Agrobacterium), physicochemical methods such as electroporation, floral dip methods, particle or ballistic bombardment, microinjection, whiskers technology, pollen tube transformation, calcium-phosphate-mediated transformation, nanoparticle-mediated transformation, polymer-mediated transformation including cyclodextrin-mediated and polyethylene glycol-mediated transformation, sonication, infiltration, as well as any other electrical, chemical, physical (mechanical) and/or biological mechanism that results in the introduction of nucleic acid into a cell, including any combination thereof.

In some embodiments of the invention, transformation of a cell comprises nuclear transformation. In other embodiments, transformation of a cell comprises plastid transformation (e.g., chloroplast transformation). In still further embodiments, the recombinant nucleic acid construct of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013))

A nucleotide sequence therefore can be introduced into a host organism or its cell in any number of ways that are well known in the art. The methods of the invention do not depend on a particular method for introducing one or more nucleotide sequences into the organism, only that they gain access to the interior of at least one cell of the organism. Where more than one polynucleotide is to be introduced, they can be assembled as part of a single nucleic acid construct, or as separate nucleic acid constructs, and can be located on the same or different nucleic acid constructs. Accordingly, the polynucleotides can be introduced into the cell of interest in a single transformation event, or in separate transformation events, or, alternatively, where relevant, a nucleotide sequence can be incorporated into a plant, as part of a breeding protocol.

Spacer sequences are used to guide the recombinant nucleic acid constructs of the invention or the co-opted endogenous CRISPR-Cas machinery of the target organism (e.g., Cascade complex) to the target sequences and are as described herein. Target sequences useful for modifying the genome of an organism or a cell thereof, useful for modifying the expression of a gene in an organism or a cell thereof, or useful for screening or killing of cells in a population may be any nucleic acid sequence (e.g., genomic sequence (e.g., an essential, a non-essential, expendable, non-expendable genomic sequence)) that is located immediately adjacent to the 3′ end of a PAM sequence (e.g., 5′-TTTA-3′, 5′-ATC-3′, and/or 5′-ATCN-3′ (N=A, T, C, or G; e.g., 5′-ATCG-3′ and/or 5′-ATCA-3′). In some embodiments, the target sequences may be conserved among the one or more cells within a population of cells. In some embodiments, the target sequence may be an essential and/or non-expendable genomic sequence that is located immediately adjacent (3′) to a PAM as defined herein (e.g., 5′-TTTA-3′, 5′-ATC-3′ and/or 5′-ATCN-3′ (N=A, T, C, or G; e.g., 5′-ATCG-3′ and/or 5′-ATCA-3′)) and that is conserved among the one or more cells within the population of cells (e.g., in a population of bacterial and/or archaeal cells). In some embodiments of the invention, the PAM may comprise, consist essentially of, or consist of a sequence of 5′-TTTA-3′, 5′-ATC-3′ and/or 5′-ATCN-3′ (N=A, T, C, or G, e.g., 5′-ATCG-3′ and/or 5′-ATCA-3′) (located immediately adjacent to and 5′ of the protospacer).

In some embodiments, targeting of a genomic sequence may result in a cell being edited or the expression of a targeted gene being altered. In some embodiments, targeting of a genomic sequence may result in a cell dying (killing), or the cell may survive by avoiding being targeted (by the recombinant nucleic acid constructs of the invention (e.g., CRISPR) by the presence of a mutation in the genomic sequence or by the cell losing the targeted genomic sequence (screening/selecting). Thus, the present invention may be used to identify natural (or induced) variants within a population that do not comprise the targeted genomic sequence and therefore survive.

Accordingly, in some embodiments, a recombinant nucleic acid construct of the invention may target, for example, coding regions, non-coding regions, intragenic regions, and intergenic regions. In some embodiments, a recombinant nucleic acid construct of the invention when used, for example, for killing may target, for example, a conserved coding region, a conserved non-coding region, a conserved intragenic region, and/or a conserved intergenic region. In some embodiments, a target sequence is located on a chromosome. In some embodiments, a target sequence is located on an extrachromosomal nucleic acid.

As used herein, “extrachromosomal nucleic acid” refers to select nucleic acids in eukaryotic cells such as in a mitochondrion, a plasmid, a plastid (e.g., chloroplast, amyloplast, leucoplast, proplastid, chromoplast, etioplast, elaiosplast, proteinoplast, tannosome), and/or an extrachromosomal circular DNA (eccDNA)). In some embodiments, an extrachromosomal nucleic acid may be referred to as “extranuclear DNA” or “cytoplasmic DNA.”

In some embodiments, a plasmid may be targeted (e.g., the target sequence is located on a plasmid), for example, for plasmid curing to eliminate undesired DNA like antibiotic resistance genes or virulence factors (e.g., a plasmid in a bacterium or an archaeon). In some embodiments, a bacterial or archaeal pathogenic trait (e.g., chromosomally-carried genes encoding an antibiotic resistance marker, a toxin, or a virulence factor) may be targeted to be removed or inactivated.

In some embodiments, a target sequence may be located in a gene, which can be in the upper (sense, coding) strand or in the bottom (antisense, non-coding) strand. In some embodiments, a target sequence may be located in an intragenic region of a gene, optionally located in the upper (sense, coding) strand or in the bottom (antisense, non-coding) strand. In some embodiments, a gene that is targeted by constructs of this invention may encode a transcription factor or a promoter. In some embodiments, a gene that is targeted may encode non-coding RNA, including, but not limited to, eukaryotic miRNA, siRNA, piRNA (piwi-interacting RNA) and lncRNA (long non-coding RNA). In some embodiments, a target sequence may be located in an intergenic region, optionally in the upper (plus) strand or in the bottom (minus) strand. In some embodiments, a target sequence may be located in an intergenic region wherein the DNA is cleaved and a gene inserted that may be expressed under the control of the promoter of the previous open reading frame.

In some embodiments, a target sequence may be located on a mobile genetic element (e.g., a transposon, a plasmid, a bacteriophage element (e.g., Mu), a group I and group II intron). Thus, for example, mobile genetic elements located in the chromosome or transposons may be targeted to force the mobile elements to jump out of the chromosome.

In some embodiments, a target sequence may be a highly conserved gene, which may carry out essential biological functions and be part of the core genome (e.g., glycolysis genes, DNA replication gene, transcription and translation machinery).

Non-limiting examples of a target sequence that may be used with the method of this invention (e.g., editing/modifying, killing, selecting, and the like) can include a region of consecutive nucleotides within a virulence gene, a prophage gene, an IS element, a transposon, a redundant gene, an accessory/non-core gene, and/or within a mobile genetic element or an expandable genomic island.

In some embodiments, a target sequence may be located in a chromosome or in a plasmid in a bacterium. In some embodiments, a target sequence is not on a plasmid. In some embodiments, a target sequence may be an essential and/or non-expendable, or a non-essential and/or expendable, genomic sequence located on a chromosome. In some embodiments, a target sequence may be an essential and/or non-expendable genomic sequence located on a chromosome. In some embodiments, the target sequence is a conserved sequence that is found within a particular bacterial species or strain of bacterial species. A “conserved sequence” as used herein means a sequence that is found, for example, across a species or within many strains within a species. Use of a conserved sequence as a target sequence (in a spacer) allows one to target that group of bacteria related by the conserved sequence. Targeting conserved genetic sequences can be advantageous because it allows one to design or “tune” CRISPR targeting spacers that allow selective killing of bacterial cells within a population based on various levels of bacterial relatedness. For example, targeting conserved genetic sequences within a species provides the ability to selectively kill multiple strains of a species. “Distinct genetic content” as used herein means that the sequence targeted is found in one strain or species and not within in a different strain or species that is present in a population of bacteria, thereby providing for selective killing by killing only the bacteria in the population that comprises the distinct genetic content.

A target organism useful with this invention may be any organism. In some embodiments, a target organism may be a prokaryote or a eukaryote. In some embodiments, a target organism may be a bacterium, an archaeon, a fungus, a plant, or an animal (e.g., a mammal, a bird, a reptile, an amphibian, a fish, an arthropod (an insect or a spider), a nematode, a mollusk, etc.). In some embodiments, the target organism may be a probiotic bacterium. In some embodiments, the target organism may be a Clostridium spp., optionally a commensal Clostridium spp. In some embodiments, the target organism may be Clostridium bolteae (e.g., C. bolteae DSM15670 (BAA-613), C. bolteae WAL14578), Clostridium clostridioforme (e.g., C. clostridioforme WAL7855, C. clostridioforme 2149FAA, C. clostridioforme YL32, C. clostridioforme NCTC11224), Clostridium scindens (e.g., C. scindens ATCC 35704, C. scindens VE20205).

In some embodiments, the invention further comprises recombinant or modified cells or organisms produced by the methods of the invention, comprising the recombinant nucleic acid constructs of the invention, and/or the recombinant plasmid, bacteriophage, and/or retrovirus comprising the recombinant nucleic acid constructs of the invention, and/or the genome modifications and/or modifications in expression generated by the methods of the invention. In some embodiments, the recombinant or modified cell or organism may be a prokaryotic cell or a eukaryotic cell, optionally a bacterial cell, an archaeon cell, a fungal cell, a plant cell, an animal cell, a mammalian cell, a fish cell, a nematode cell, or an arthropod cell. In some embodiments, a recombinant or modified cell of the invention may be a Clostridium spp. cell. The term “recombinant cell” or “recombinant organism” as used herein refers to a cell or organism that is stably transformed with at least one nucleic acid construct of this invention. A cell or organism may also be transiently transformed with the at least one nucleic acid construct of this invention. In some embodiments, a cell or organism that is transformed with at least one nucleic acid construct of this invention may be edited, killed, selected, and the like, as described herein. A “modified” cell or organism is a cell or organism that is edited as described herein. In some embodiments, a modified cell or organism that is modified using the methods of this invention is not stably transformed with a nucleic acid construct of this invention. In some embodiments, a cell or organism that is transformed with at least one nucleic acid construct of this invention may be stably transformed (e.g., recombinant) or may be transiently transformed with the at least one nucleic acid construct.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1. CRISPR-Cas System Identification and Characterization in Erysipelatoclostridium Ramosum and Clostridium Spp. 1141A1FAA

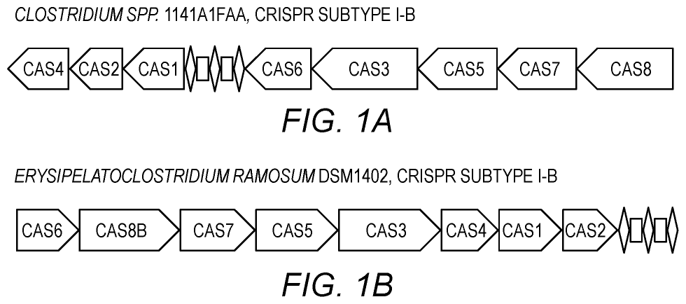

FIGS. 1A and 1B show the results of the characterization of the E. ramosum and C. spp. 1141A1FAA Cascade complex in which E. ramosum is shown to comprise a Cas3, Cas6, a Cas8, Cas7 and a Cas5 in addition to the spacer acquisition polypeptides Cas1, Cas2, and Cas4 (FIG. 1B). C. spp. 1141A1FAA is shown to comprise a Cas3, Cas6, a Cas8, Cas7 and a Cas5 in addition to the spacer acquisition polypeptides Cas1, Cas2, and Cas4 (FIG. 1A). The CRISPR-Cas system subtype I-B of E. ramosum DSM1402 (Cas6-Cas8-Cas7-Cas5b-Cas3-Cas4-Cas1-Cas2) was compared with the canonical subtype I-B from Clostridium kluyveri DSM555 using MUSCLE algorithm for the nucleotide sequence alignment. The comparison showed that the nucleotide sequences of the CRISPR-Cas system subtype I-B of E. ramosum share less than 56% sequence identity with the nucleotide sequences from the canonical subtype I-B from Clostridium kluyveri DSM555. In addition, the E. ramosum Cas3 amino acid sequence was found to share less than 40% sequence identity with the Cas3 amino acid sequence of the Clostridium kluyveri DSM555 canonical subtype I-B system. This further demonstrates the distinctiveness of this newly characterized CRISPR-Cas system.

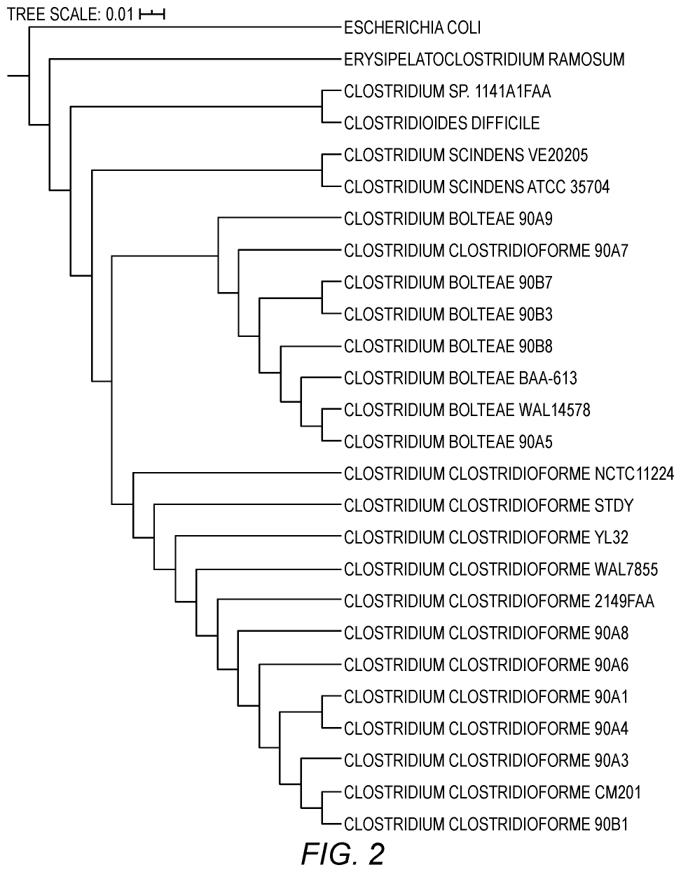

A phylogenomic analysis comparing the different Clostridium species of this invention and including E. coli, C. difficile and Erysipelatoclostridium ramosum is provided in FIG. 2 shows the phylogenetic distance among this species.

### Example 2. PAM Prediction

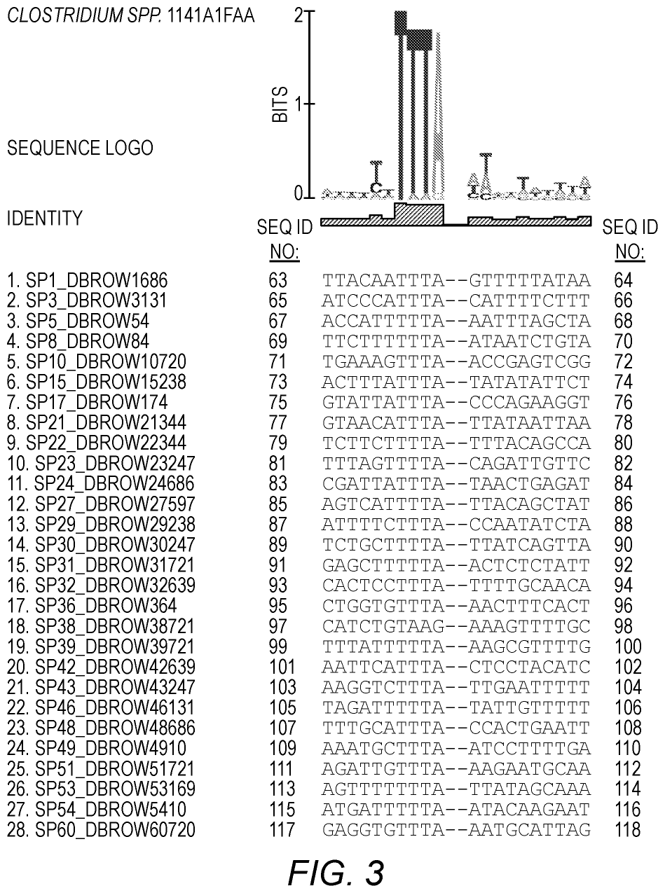

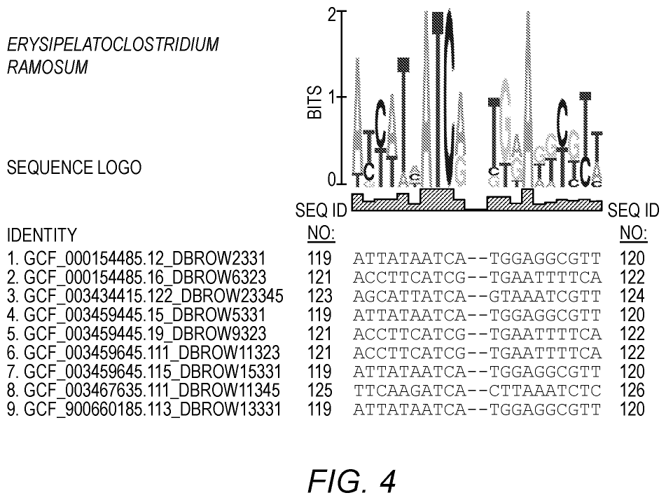

The CRISPR spacers were extracted from the CRISPR array of each of the strains described in this invention, and a blastn was performed against different NCBI databases. The spacer-protospacer positive matches obtained were used to extract 10 nt of the adjacent (upstream and downstream) regions of the protospacer to elucidate the PAM sequence. The PAM sequences for the CRISPR-Cas system Type I-B locus from C. spp. 1141A1FAA and E. ramosum were predicted as shown in FIGS. 3 and 4, respectively.

### Example 3. Bacterial Strains and Growth Conditions

The bacterial strains listed in this invention are generally grown in broth or agar media, at anaerobic conditions and 37° C. for 2-5 days. The media to be used is species dependent and in some cases even strain dependent. The media used can include but not limited to Brain Heart Infusion (BHI) with or without 0.05-0.5% (w/v) L-cysteine, Reinforced Clostridial Medium (RCM) with or without 0.05-0.5% (w/v) as examples.

### Example 4. Validating the Functionality of the E. ramosum Type I CRISPR-Cas System

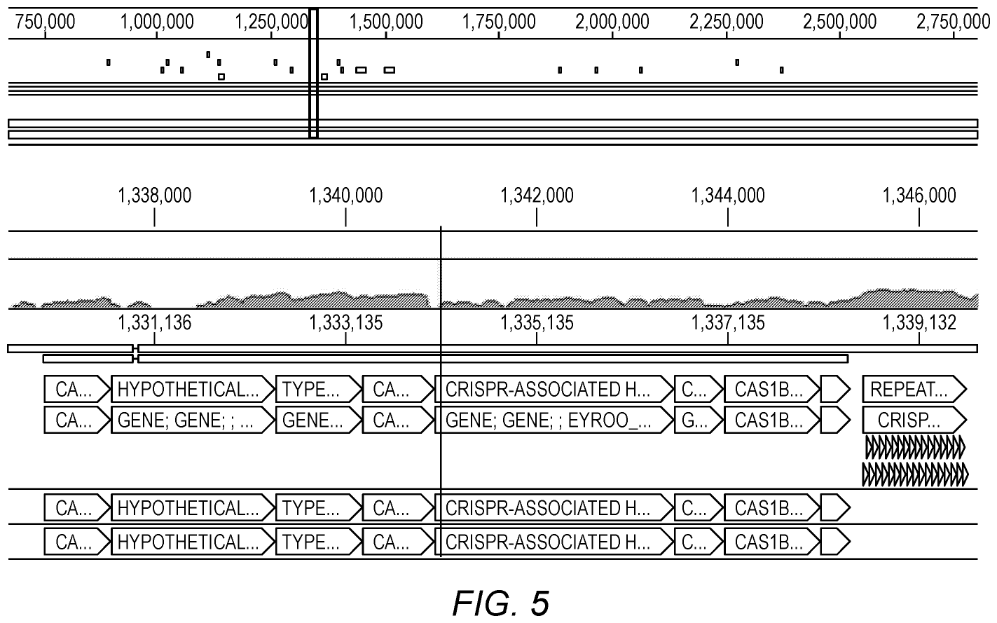

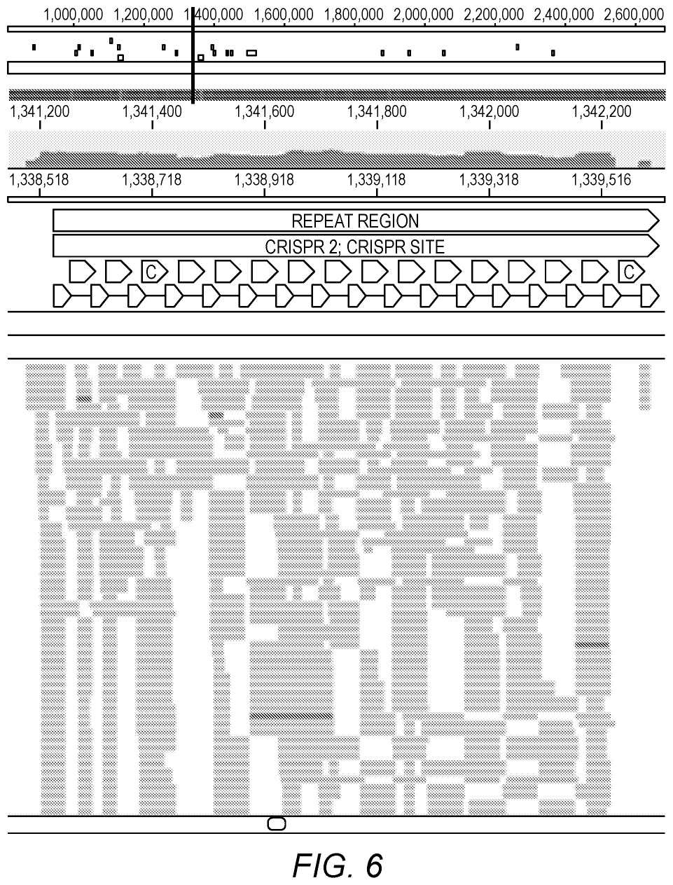

In order for CRISPR-Cas systems to be functional, it is necessary to have transcription of the cas genes to form the Cascade complex and transcription of the CRISPR array to generate mature CRISPR RNAs (crRNAs) that can guide the Cas machinery to the complementary sequence. We determined cas and CRISPR array transcriptional profiles in the native host to show activity of the endogenous E. ramosum type I CRISPR-Cas system, revealing cas transcription and the boundaries and sequence of the corresponding mature crRNA (See, FIG. 5 and FIG. 6). Sequencing was performed by UIUC using Illumina paired ends, and data was assembled, mapped and analyzed in Geneious Prime using the Geneious mapper.

### Example 5. The Mature E. ramosum crRNA

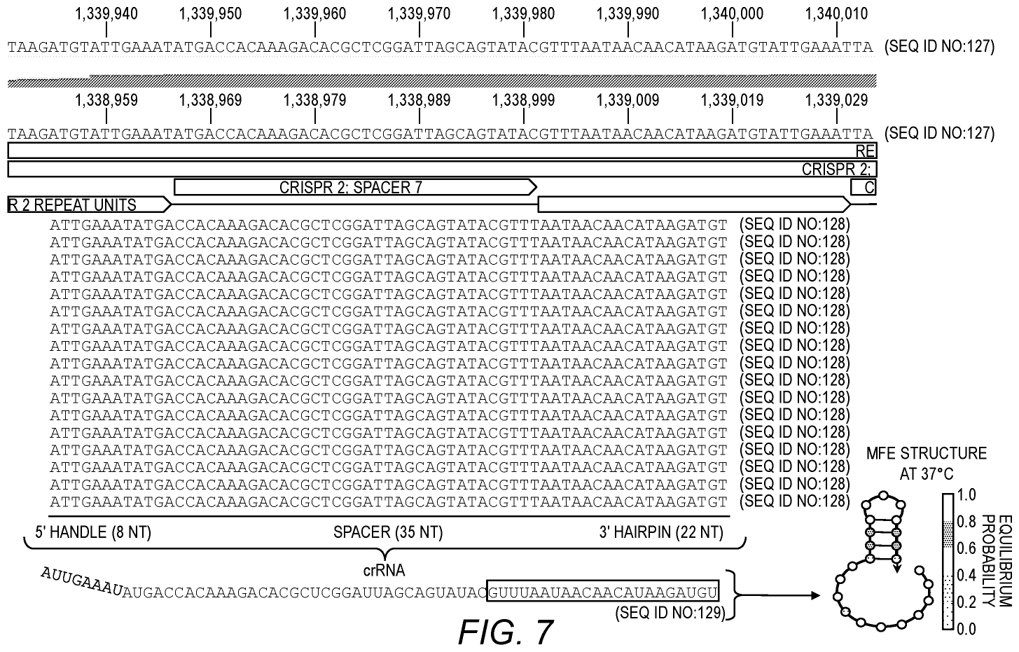

The composition, structure and boundaries of the mature E. ramosum crRNA was determined. The mature E. ramosum crRNA comprised of a full CRISPR spacer (can range between 34 nt and 37 nt) flanked by two sections of the CRISPR repeat, the 5′ handle (comprised of the 8 nt of the 3′ portion of the CRISPR repeat) and the 3′ hairpin (comprised of the 22 nt of the 5′ portion of the CRISPR repeat, which carries the palindrome within the CRISPR, and reveals processing at the base of the hairpin to generate the mature crRNA from the pre-crRNA full transcript) (see, FIG. 7). The hairpin structure shown in FIG. 7 was visualized in NUPACK. For CRISPR locus #2, spacer length can vary between about 34 to 37 nucleotides.

### Example 6. PAM Selection and deGFP Protospacer Sequence Determination

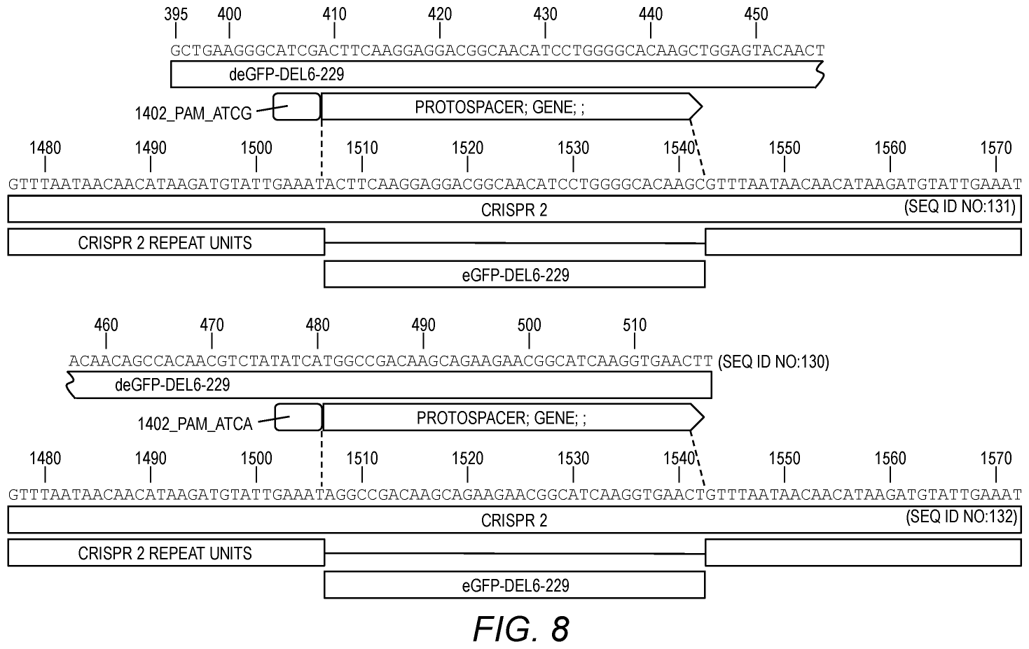

FIG. 8 provides a schematic of an example targeting CRISPR array with a spacer that is complementary to the sequence as shown in FIG. 8, enabling the genesis of a mature crRNA with a 36 nt targeting spacer flanked by the E. ramosum CRISPR repeat sections. The upper panel of FIG. 8 shows the target with a ATCG PAM flanking the 5′ edge of the protospacer. The lower panel of FIG. 8 shows the target with a ATCA PAM flanking the 5′ edge of the protospacer. A PAM sequence logo is shown in FIG. 4.

### Example 7. Transcriptional Control in Cell-Free Transcription-Translation (TXTL)

A transcription (TX)-translation (TL) platform, TXTL, was used with a mastermix, which consists of a cell-free extract enabling in vitro analysis of CRISPR effectors (Daicel Arbor Biosciences). This system is based on RNA polymerase sigma factor 70 (˜70) for recognition of promoters on synthetic plasmids engineered to provide Cas proteins, the corresponding guide crRNAs and the target sequences. Reactions were carried out in small volumes (5 μl) in scalable formats (96-well plates) with fluorescence outputs that show Cas protein activity (e.g., binding to the target sequence blocking transcription and preventing GFP fluorescence). In this example, we provided the E. ramosum Cas machinery on a plasmid in combination with a CRISPR array comprising a spacer targeting the promoter sequence of the GFP gene. Transcription was prevented by Cascade binding to the complementary sequence and preventing transcription, showing Cascade programmability by the engineered CRISPR array.

A GFP fluorescence assay was used to show targeting by the E. ramosum Cascade-crRNA complex. The targeting was revealed by lowering of GFP transcription due to binding to the target sequence (complementary to the CRISPR spacer) and percent repression was calculated in the following manner:

$$1 - \frac{targ eting endpoint}{non - targeting endpoint} * 100$$

### Example 8. TXTL Genetic Circuit/Reaction

The E. ramosum Cascade set of cas genes (see, FIG. 1, FIG. 5) is provided in combination with a CRISPR array comprising two repeats flanking a targeting spacer for a TXTL genetic circuit/reaction.

The TXTL experimental set up was as follows:
1. TXTL master mix-positive control
   myTXTL Sigma 70 Master Mix (75 uL)—contains Sigma 70 Master Mix and pTXTL-T70a(2)—deGFP HP control plasmid
   p70a-deGFP—2 nM
   P70a-T7RNAP—1 nM
   IPTG—1 nM
   H2O
2. Sample prep
   Targeting plasmid: E_ramosum_1402_PAM_ATCG (0.5 nM concentration) or E_ramosum_1402_PAM_ATCA (0.5 nM concentration)
   Non-targeting plasmid—negative control: E_ramosum_1402_NT (0.5 nM concentration)
   Each of targeting plasmid and non-targeting plasmid contain TXTL master mix & H2O
3. Blank—positive control—used to subtract out background: myTXTL Sigma 70 Master Mix only
4. Reaction
   Plate reader—BMG Labtech FLUOstar Omega
   deGFP RFU measured every 10 mins for 16 hrs, 97 cycles total
29° C. reaction temperature

The E. coli cell-free transcription-translation (TXTL) system was used in vitro to test the functionality of the type I-B CRISPR-Cas system derived from Erysipelatoclostridium ramosum DSM 1402. Expressed in the targeting plasmid, are the multi-effector CRISPR nucleases, cas proteins cas6875b, that form the active CRISPR machinery (cascade-CRISPR associated complex for antiviral defense) needed for targeted gene repression (deGFP) in the TXTL reaction. In addition, the deGFP gRNA is expressed in the targeting plasmid which binds complementary to the protospacer region next to the PAM ATCG or ATCA sequence in the p70a-deGFP plasmid.

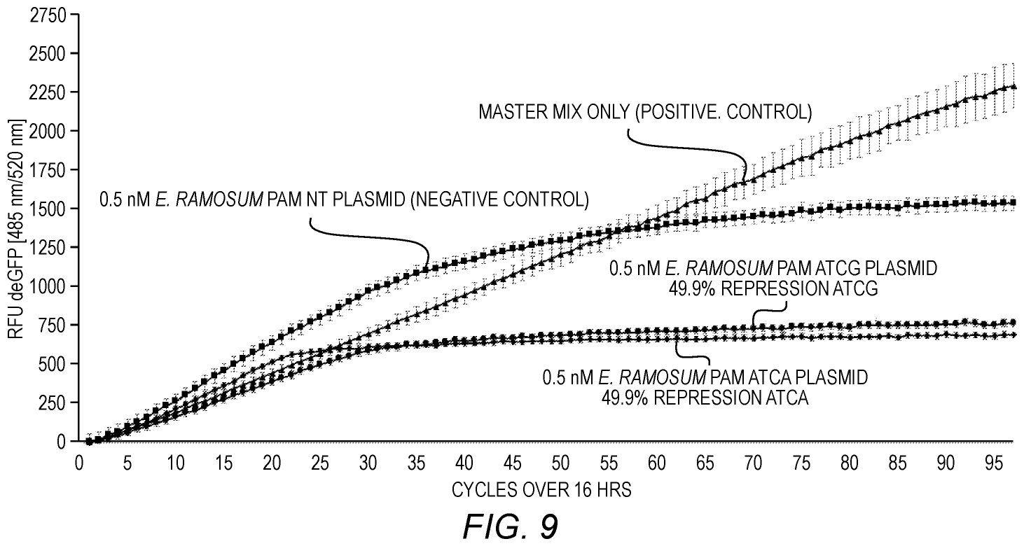

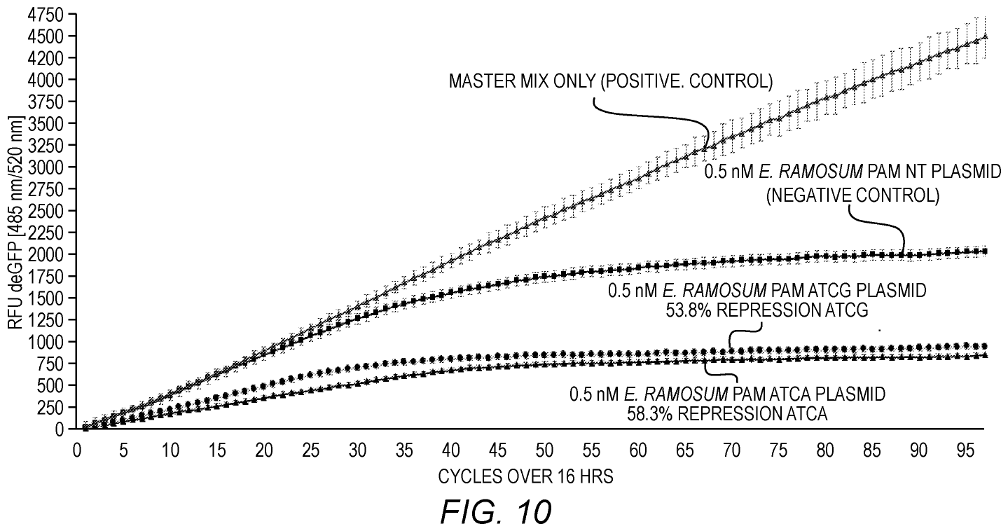

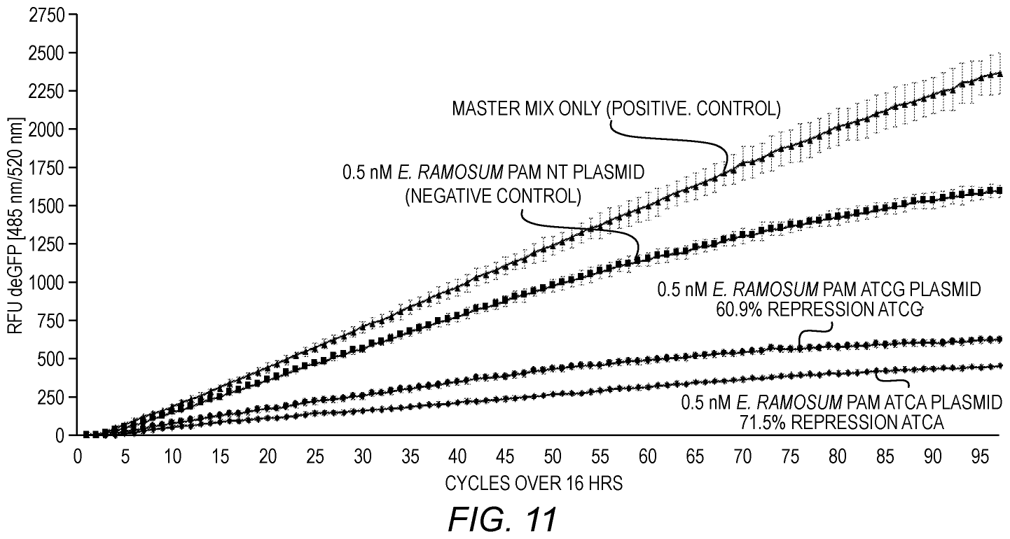

The results of the TXTL experiments are shown in FIGS. 9-11. FIG. 9 provides the results of round 1 testing of 0.5 nM E. ramosum PAM ATCA or ATCG plasmid using a non-targeting plasmid as a neg. control. FIG. 10 provides the results of round 2 testing of 0.5 nM E. ramosum PAM ATCA or ATCG plasmid using a non-targeting plasmid as a neg. control. FIG. 11 provides the results of round three testing of 0.5 nM E. ramosum PAM ATCA or ATCG plasmid using a non-targeting plasmid as a neg. control.

Using the in vitro TXTL system, 49.9%, 53.8%, and 60.9% deGFP repression was achieved at a 0.5 nM concentration using the PAM ATCG targeting plasmid. A 54.9%, 58.3%, and 71.5% deGFP repression was achieved at a 0.5 nM concentration using the PAM ATCA targeting plasmid. (see, FIGS. 9-11). The TXTL reaction confirmed system activity and efficient repression using the endogenous type I-B CRISPR cascade from E. ramosum DSM 1402 when provided with a guide RNA that matched a target (deGFP) positioned next to the predicted PAM (ATCG or ATCA).

The foregoing is illustrative of the present invention and is not to be construed as limiting thereof. The invention is defined by the following claims, with equivalents of the claims to be included therein.

That which is claimed is:

1. A recombinant nucleic acid construct comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and the spacer sequence is complementary to a target sequence (protospacer) in a nucleic acid of an organism, wherein the target sequence is located immediately adjacent (3′) to a protospacer adjacent motif (PAM).

2. A protein-RNA complex comprising:
(a) a Type I-B CRISPR associated complex for antiviral defense complex (Cascade complex) comprising a Cas6 polypeptide comprising the amino acid sequence of SEQ ID NO: 1 or SEQ ID NO:19, a Cas8 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:2 or SEQ ID NO:20, a Cas7 polypeptide having at least 80% sequence identity to the amino acid sequence of SEQ ID NO:3 or SEQ ID NO:21, and a Cas5 polypeptide having at least 80% sequence identity to the amino acid sequence of SEQ ID NO: 4 or SEQ ID NO:22, and/or a Cas3 polypeptide having at least 80% sequence identity to the amino acid sequence of SEQ ID NO:5 or SEQ ID NO:23; and
(b) a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and the spacer sequence is complementary to a target sequence (protospacer) in a nucleic acid of a target organism, wherein the target sequence is located immediately adjacent (3′) to a protospacer adjacent motif (PAM).

3. The recombinant nucleic acid construct of claim 1 or the protein-RNA complex of claim 2, wherein each of the one or more spacer sequences is linked at its 3′-end to a repeat sequence or portion thereof.

4. The recombinant nucleic acid construct of claim 1 or claim 3 or the protein-RNA complex of claim 2 or claim 3, wherein the one or more repeat sequences comprise at least 19 consecutive nucleotides (e.g., about 19, 20, 21, 22, 23, 24, 25, 26, 27, 28, 29, 30, 31, or 32 consecutive nucleotides) having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the nucleotide sequences of SEQ ID NO:17 or SEQ ID NO 18 or any one of the nucleotide sequences of SEQ ID NOs: 35-43, optionally about 27 to 32 consecutive nucleotides or about 30 to 32 consecutive nucleotides.

5. The recombinant nucleic acid construct of any one of claim 1, 3 or 4, or the protein-RNA complex of any one of claims 2-4, wherein the PAM comprises a nucleotide sequence of 5′-TTTA-3′, 5′-ATC-3′, 5′-ATCG-3′ and/or 5′-ATCA-3′ that is immediately adjacent to and 5′ of the target sequence (protospacer).

6. The recombinant nucleic acid construct of any one of claim 1, 3, 4 or 5, further comprising a promoter operably linked to the CRISPR.

7. The recombinant nucleic acid construct of claim 6, wherein the promoter is endogenous to the repeat sequences of the CRISPR (e.g., endogenous to the repeat sequences of Erysipelatoclostridium ramosum or Clostridium spp. 1141A1FAA).

8. The recombinant nucleic acid construct of claim 6, wherein the promoter is a heterologous to the repeat sequences.

9. The recombinant nucleic acid construct of any one of claims 6-8, wherein the promoter comprises the nucleotide sequence of any of SEQ ID NOs: 44-52.

10. The recombinant nucleic acid construct of any one of claim 1 or 3-9, further comprising a terminator sequence operably linked to the CRISPR.

11. The recombinant nucleic acid construct of claim 10, wherein the terminator sequence is a Rho-independent terminator sequence, a Erysipelatoclostridium ramosum terminator sequence, or a Clostridium spp. 1141A1FAA terminator sequence.

12. The recombinant nucleic acid construct of claim 10 or claim 11, wherein the terminator comprises the nucleotide sequence of any of SEQ ID NOs: 53-61.

13. The recombinant nucleic acid construct of any one of claim 1 or 3-12 or the protein-RNA complex of any one of claims 2-5, wherein the spacer sequence is 100% complementary to the target sequence.

14. The recombinant nucleic acid construct of any one of claim 1 or 3-13 or the protein-RNA complex of any one of claims 2-5, wherein the spacer sequence is about 80% complementary to the target sequence.

15. The recombinant nucleic acid construct of any one of claim 1 or 3-14 or the protein-RNA complex of any one of the claims 2-5, 13 or 14, wherein the one or more spacer sequence(s) each have a length of about 25 nucleotides to about 60 nucleotides, optionally about 30 nucleotides to about 40 nucleotides, about 32 nucleotides to about 40 nucleotides, or about 34, 35, 36 or 37 nucleotides.

16. The recombinant nucleic acid construct of any one of claim 1 or 3-15 or the protein-RNA complex of any one of claims 2-6 or 13-15, wherein at least two of the one or more spacer sequence(s) comprise nucleotide sequences that are complementary to different target sequences.

17. The recombinant nucleic acid construct of any one of claim 1 or 3-16 or the protein-RNA complex of any one of claim 2-6 or 13-16, wherein the one or more spacer sequence(s) each comprise a 5′ region and a 3′ region, wherein the 5′ region comprises a seed sequence and the 3′ region comprises a remaining portion of the one or more spacer sequence(s).

18. The recombinant nucleic acid construct of claim 17 or the protein-RNA complex of claim 17, wherein the seed sequence comprises the first 8 nucleotides of the 5′ end of each of the one or more spacer sequence(s), and is fully complementary to the target sequence, and the remaining portion of the one or more spacer sequence(s) is at least about 80% complementary to the target sequence.

19. The recombinant nucleic acid construct of any one of claim 1 or 3-18, or the protein-RNA complex of any one of claim 2-6 or 13-18, wherein the target sequence is located in a gene, optionally in the upper (sense, coding) strand or in the bottom (antisense, non-coding) strand.

20. The recombinant nucleic acid construct of claim 19 or protein-RNA complex of claim 19, wherein the target sequence is located in an intragenic region of the gene (e.g., an intron), optionally located in the upper (sense, coding) strand or in the bottom (antisense, non-coding) strand.

21. The recombinant nucleic acid construct of any one of claim 1 or 3-18 or the protein-RNA complex of any one of claim 2-6 or 13-18, wherein the target sequence is located in an intergenic region, optionally in the upper (plus) strand or in the bottom (minus) strand.

22. The recombinant nucleic acid construct of any one of claim 1, or 3-21 or the protein-RNA complex of any one of claim 2-6 or 13-21, wherein the target sequence is located on a chromosome.

23. The recombinant nucleic acid construct of any one of claim 1 or 3-21 or the protein-RNA complex of any one of claim 2-6 or 13-21, wherein the target sequence is located on a mobile element.

24. The recombinant nucleic acid construct of any one of claim 1 or 3-21 or the protein-RNA complex of any one of claims 2-20, wherein the target sequence is located on extrachromosomal nucleic acid.

25. The recombinant nucleic acid construct of any one of claim 1, or 3-21, 23 or 24 the protein-RNA complex of any one of claim 2-21, 23 or 24, wherein the target sequence is located on a plasmid.

26. The recombinant nucleic acid construct of any one of claims 1, 3-25 or the protein-RNA complex of any one of claim 2-6 or 13-25, wherein the gene encodes a transcription factor or a promoter.

27. The recombinant nucleic acid construct of any one of claim 1, 3-19 or 21-25 or the protein-RNA complex of any one of claim 2-5, 13-19 or 21-25, wherein the gene encodes non-coding RNA (e.g., miRNA, siRNA, piRNA (piwi-interacting RNA) and lncRNA (long non-coding RNA)).

28. The recombinant nucleic acid construct of any one of claim 1 or 3-27 or the protein-RNA complex of any one of claim 2-6 or 13-27, wherein the target organism is a prokaryote or a eukaryote.

29. A vector encoding the recombinant nucleic acid of any one of claim 1 or 3-28.

30. The vector of claim 29, further comprising a recombinant nucleic acid encoding a Type I-B CRISPR associated complex for antiviral defense complex (Cascade complex) comprising a Cas6 polypeptide, a Cas8 polypeptide, a Cas7 polypeptide, a Cas5 polypeptide, and a Cas3 polypeptide.

31. The vector of claim 30, wherein the Cas6 polypeptide comprises a sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1 or SEQ ID NO:19, the Cas8 polypeptide comprises a sequence having at least 80% sequence identity to the amino acid sequence of SEQ ID NO:2 or SEQ ID NO:20, the Cas7 polypeptide comprises a sequence having at least 80% sequence identity to the amino acid sequence of SEQ ID NO:3 or SEQ ID NO: 21, the Cas5 polypeptide comprises a sequence having at least 80% sequence identity to the amino acid sequence of SEQ ID NO:4 or SEQ ID NO:22, and the Cas3 polypeptide comprises a sequence having at least 80% sequence identity to the amino acid sequence of SEQ ID NO:5 or SEQ ID NO:23.

32. The vector of any one of claims 29-31, wherein the vector is a plasmid, bacteriophage, transposon, phagemid, and/or retrovirus.

33. A cell comprising the recombinant nucleic acid of any one of claim 1 or 3-28, the protein-RNA complex of any one of claims 2-6, 13-28, or the vector of claim 29-32.

34. The cell of claim 33, wherein the Cas6 polypeptide, the Cas8 polypeptide, the Cas7 polypeptide, the Cas5 polypeptide, and/or the Cas3 polypeptide are codon optimized for expression in the cell.

35. The cell of claim 33 or claim 34, wherein the cell is a plant cell, bacteria cell, fungal cell, mammalian cell, insect cell, or archaeon cell.

36. A method of modifying (editing) the genome of a target organism, comprising introducing into the target organism or a cell of the target organism
(a) a recombinant nucleic acid construct comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and the spacer sequence is complementary to a target sequence (protospacer) in a target nucleic acid of a target organism, wherein the target sequence is located immediately adjacent (3′) to a protospacer adjacent motif (PAM);
(b) a recombinant nucleic acid construct encoding: a Type I-B CRISPR associated complex for antiviral defense complex (Cascade complex) comprising:
a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO: 1 or SEQ ID NO:19,
a Cas8 polypeptide having at least 80% sequence identity to the amino acid sequence of SEQ ID NO:2 or SEQ ID NO:20,
a Cas7 polypeptide having at least 80% sequence identity to the amino acid sequence of SEQ ID NO:3 or SEQ ID NO:21, and
a Cas5 polypeptide having at least 80% sequence identity to the amino acid sequence of SEQ ID NO:4 or SEQ ID NO:22, and
a Cas3 polypeptide having at least 80% sequence identity to the amino acid sequence of SEQ ID NO:5 or SEQ ID NO:23; and
(c) a repair template, thereby modifying the genome of the target organism.

37. A method of modifying the genome of a bacterial cell that comprises an endogenous Type I-B CRISPR-Cas system, comprising introducing into the bacterial cell
(a) a recombinant nucleic acid construct comprising a Clustered Regularly Interspaced Short Palindromic Repeats (CRISPR) comprising one or more repeat sequences and one or more spacer sequence(s), each of the one or more spacer sequences is linked at least at its 5′-end to a repeat sequence or portion thereof, and each of the one or more spacer sequence(s) is complementary to a target sequence (protospacer) in a nucleic acid of the bacterial cell, wherein the target sequence is located immediately adjacent (3′) to a protospacer adjacent motif (PAM); and
(b) a repair template, thereby modifying the genome of the bacterial cell.

38. The method of claim 36 or claim 37, wherein the target organism or bacterial cell is a cell of a commensal Clostridium spp.

39. A method of altering the expression (repressing expression/overexpression) of a target gene in a target organism, comprising introducing into the target organism or a cell of the target organism
(a) a recombinant nucleic acid construct comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and the spacer sequence is complementary to a target sequence (protospacer) in a nucleic acid of a target organism, wherein the target sequence is located immediately adjacent (3′) to a protospacer adjacent motif (PAM); and
(b) a recombinant nucleic acid construct encoding a Type I-B CRISPR associated complex for antiviral defense complex (Cascade complex) comprising:
a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO: 1 or SEQ ID NO:19,
a Cas8 polypeptide having at least 80% sequence identity to the amino acid sequence of SEQ ID NO:2 or SEQ ID NO:20,
a Cas7 polypeptide having at least 80% sequence identity to the amino acid sequence of SEQ ID NO:3 or SEQ ID NO:21,
a Cas5 polypeptide having at least 80% sequence identity to the amino acid sequence of SEQ ID NO:4 or SEQ ID NO:22, thereby altering expression of the target gene in the cell of the target organism.

40. A method of screening for a variant cell of a target organism, the method comprising
(a) introducing into a population of cells from (or of) the target organism:
(i) a recombinant nucleic acid construct comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and the spacer sequence is complementary to a target sequence (protospacer) in a target nucleic acid of the organism, wherein the target sequence is located immediately adjacent (3′) to a protospacer adjacent motif (PAM);
(ii) a recombinant nucleic acid construct encoding a Type I-B CRISPR associated complex for antiviral defense complex (Cascade complex) comprising: a recombinant nucleic acid construct encoding a Type I-B CRISPR associated complex for antiviral defense complex (Cascade complex) comprising:
a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1 or SEQ ID NO:19,
a Cas8 polypeptide having at least 80% sequence identity to the amino acid sequence of SEQ ID NO:2 or SEQ ID NO:20,
a Cas7 polypeptide having at least 80% sequence identity to the amino acid sequence of SEQ ID NO:3 or SEQ ID NO:21, and
a Cas5 polypeptide having at least 80% sequence identity to the amino acid sequence of SEQ ID NO:4 or SEQ ID NO:22, and
a Cas3 polypeptide having at least 80% sequence identity to the amino acid sequence of SEQ ID NO:5 or SEQ ID NO:23;
wherein the recombinant nucleic acid construct comprising a CRISPR and the recombinant nucleic acid construct encoding a Cascade complex each comprise a polynucleotide encoding a polypeptide conferring resistance to a selection agent, thereby producing a subpopulation of cells of the population of cells; and
(b) selecting from the subpopulation of cells produced in (a) one or more cells that are resistant to the selection agent, thereby selecting one or more variant cells that do not comprise the target sequence and are not killed.

41. A method of screening for variant bacterial cells comprising an endogenous Type I-B CRISPR-Cas system, the method comprising
(a) introducing into a population of bacterial cells a recombinant nucleic acid construct comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and the spacer sequence is complementary to a target sequence (protospacer) in a nucleic acid of the bacteria wherein the target sequence is not present in the variant cell and the target sequence is located immediately adjacent (3′) to a protospacer adjacent motif (PAM); and
wherein the recombinant nucleic acid construct comprising a CRISPR comprises a polynucleotide encoding a polypeptide conferring resistance to a selection agent, thereby killing transformed cells comprising the target sequence and producing a subpopulation of bacterial cells; and
(b) selecting from the subpopulation of bacterial cells produced in (a) one or more bacterial cells that are resistant to the selection agent, thereby selecting one or more variant bacterial cells that do not comprise the target sequence and are not killed.

42. The method of claim 41, wherein the population of bacterial cells is a population of commensal Clostridium spp. cells.

43. A method of killing one or more cells in a population of bacterial and/or archaeal cells, the method comprising introducing into the one or more cells of the population of bacterial and/or archaeal cells:
(a) a recombinant nucleic acid construct comprising a Clustered Regularly Interspaced Short Palindromic Repeats (CRISPR) comprising one or more repeat sequences and one or more spacer nucleotide sequence(s),
wherein each of the one or more spacer sequences comprises a 3′ end and a 5′ end and is linked at least at its 5′-end to a repeat sequence or portion thereof, and each of the one or more spacer sequences is complementary to a target sequence (protospacer) in the genome of the bacterial and/or archaeal cells of the population, and the target sequence is a genomic sequence that is conserved among the one or more cells within the population of bacterial and/or archaeal cells and the target sequence is located immediately adjacent (3′) to a protospacer adjacent motif (PAM); and
(b) a recombinant nucleic acid construct encoding a Type I-B CRISPR associated complex for antiviral defense complex (Cascade complex) comprising: a recombinant nucleic acid construct encoding a Type I-B CRISPR associated complex for antiviral defense complex (Cascade complex) comprising:
a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1 or SEQ ID NO:19,
a Cas8 polypeptide having at least 80% sequence identity to the amino acid sequence of SEQ ID NO:2 or SEQ ID NO:20,
a Cas7 polypeptide having at least 80% sequence identity to the amino acid sequence of SEQ ID NO:3 or SEQ ID NO:21, and
a Cas5 polypeptide having at least 80% sequence identity to the amino acid sequence of SEQ ID NO:4 or SEQ ID NO:22, and
a Cas3 polypeptide having at least 80% sequence identity to the amino acid sequence of SEQ ID NO:5 or SEQ ID NO:23, thereby killing one or more cells in the population of bacterial and/or archaeal cells that comprise the target sequence in their genome.

44. A method of killing one or more cells in a population of bacterial and/or archaeal cells that comprise an endogenous Type I-B Clustered Regularly Interspaced Short Palindromic Repeats (CRISPR)-Cas system, the method comprising introducing into the one or more cells of the population of bacterial and/or archaeal cells a recombinant nucleic acid construct comprising a CRISPR comprising one or more repeat sequences and one or more spacer nucleotide sequence(s),
wherein each of the one or more spacer sequences comprises a 3′ end and a 5′ end and is linked at least at its 5′ end to a repeat sequence, and each of the one or more spacer sequences is complementary to a target sequence (protospacer) in a target DNA in the one or more bacterial and/or archaeal cells of the population,
wherein the target sequence is conserved among the one or more cells within the population of bacterial and/or archaeal cells and the target sequence is located immediately adjacent (3′) to a protospacer adjacent motif (PAM),
thereby killing the one or more cells within the population of bacterial and/or archaeal cells that comprise the target sequence in their genome.

45. The method of any one of claims 36-44, wherein each of the one or more spacer sequences is linked at its 3′-end to a repeat sequence or portion thereof.

46. The method of any one of claim 36, 38, 39, 40, 42, 43 or 45, wherein the recombinant nucleic acid construct comprising a CRISPR and/or the recombinant nucleic acid construct encoding a Cascade complex are comprised in a single vector and/or expression cassette or are comprised in two or three separate vectors and/or expression cassettes.

47. The method of any one of claims 37, 38, 41, 42 or claim 44, wherein the recombinant nucleic acid construct comprising a CRISPR is comprised in an expression cassette and/or a vector.

48. The method of claim 46 or claim 47, wherein the vector is a recombinant plasmid, bacteriophage, transposon, phagemid, or retrovirus.

49. The method of any one of claim 36, 39, 40, 43, 45 or 47, wherein the recombinant nucleic acid construct comprising a CRISPR and the recombinant nucleic acid construct encoding a Cascade complex are introduced into the target organism or cell of the target organism simultaneously, separately and/or sequentially.

50. The method of claim 49, wherein the recombinant nucleic acid construct comprising the CRISPR and the recombinant nucleic acid construct encoding the Cascade complex are comprised in the same vector.

51. The method of any one of claims 36-50, wherein the recombinant nucleic acid construct comprising a CRISPR is operably linked to a promoter and/or the recombinant nucleic acid construct encoding the Cascade complex is operably linked to a promoter.

52. The method of any one of claims 36-51, wherein the recombinant nucleic acid construct comprising a CRISPR and the recombinant nucleic acid construct encoding the Cascade complex are operably linked to a single promoter or are operably linked to separate promoters.

53. The method of claim 51 or claim 52, wherein the single promoter and/or the separate promoters are endogenous or heterologous to the repeat sequences of the CRISPR (e.g., endogenous to the repeat sequences of Erysipelatoclostridium ramosum or Clostridium spp. 1141A1FAA, in any combination.

54. The method of any one of claims 51 to 53, wherein the promoter and/or the separate promoters comprise the nucleotide sequence of any of SEQ ID NOs: 44-52, or any combination thereof.

55. The method of any one of claims 36 to 54, wherein the recombinant nucleic acid construct comprising a CRISPR is operably linked to a terminator sequence and/or the recombinant nucleic acid construct encoding the Cascade complex is operably linked to a terminator sequence.

56. The method of any one of claims 36-55, wherein the recombinant nucleic acid construct comprising a CRISPR and the recombinant nucleic acid construct encoding the Cascade complex are operably linked to a single terminator sequence or are operably linked separate terminator sequences.

57. The method of claim 55 or claim 56, wherein the terminator sequence and/or the separate terminator sequences comprise a Rho-independent terminator sequence, a Erysipelatoclostridium ramosum terminator sequence, or a Clostridium spp. 1141A1FAA terminator sequence, or any combination thereof.

58. The method of any one of claims 55-57, wherein the terminator sequence and/or the separate terminator sequences comprise the nucleotide sequence of any of SEQ ID NOs: 53-61.

59. A method of modifying (editing) the genome of a target organism, comprising introducing into the target organism or a cell of the target organism a protein-RNA complex, the protein-RNA complex comprising:
(a) a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and the spacer sequence is complementary to a target sequence (protospacer) in a target nucleic acid of a target organism, wherein the target sequence is located immediately adjacent (3′) to a protospacer adjacent motif (PAM);
(b) a Cas3 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO: 5 or SEQ ID NO:23, and a Type I-B CRISPR associated complex for antiviral defense complex (Cascade complex) comprising (i) a Cas6 polypeptide having at least 80% sequence identity to the amino acid sequence of SEQ ID NO:1 or SEQ ID NO:19, a Cas8 polypeptide having at least 80% sequence identity to the amino acid sequence of SEQ ID NO:2 or SEQ ID NO: 20, a Cas7 polypeptide having at least 80% sequence identity to the amino acid sequence of SEQ ID NO:3 or SEQ ID NO:21, and a Cas5 polypeptide having at least 80% sequence identity to the amino acid sequence of SEQ ID NO:4 or SEQ ID NO:22; and
(c) a repair template, thereby modifying the genome of the target organism.

60. The method of claim 59, wherein the target organism is a bacterial cell, optionally a cell of a commensal Clostridium spp.

61. A method of altering the expression (repressing expression/overexpression) of a target gene in a target organism, comprising introducing into the target organism or a cell of the target organism a protein-RNA complex, the protein-RNA complex comprising:
(a) a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and the spacer sequence is complementary to a target sequence (protospacer) in a target nucleic acid of a target organism, wherein the target sequence is located immediately adjacent (3′) to a protospacer adjacent motif (PAM); and
(b) a Type I-B CRISPR associated complex for antiviral defense complex (Cascade complex) comprising (i) a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:1 or SEQ ID NO:19, a Cas8 polypeptide having at least 80% sequence identity to the amino acid sequence of SEQ ID NO:2 or SEQ ID NO:20, a Cas7 polypeptide having at least 80% sequence identity to the amino acid sequence of SEQ ID NO: 3 or SEQ ID NO:21, and a Cas5 polypeptide having at least 80% sequence identity to the amino acid sequence of SEQ ID NO:4 or SEQ ID NO:22, thereby altering expression of the target gene in the cell of the target organism.

62. A method of screening for a variant cell of an organism, the method comprising
(a) introducing into a population of cells from (or of) the organism a protein-RNA complex, the protein-RNA complex comprising:
(i) a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and the spacer sequence is complementary to a target sequence (protospacer) in a target nucleic acid of the organism, wherein the target sequence is located immediately adjacent (3′) to a protospacer adjacent motif (PAM);
(ii) a Cas3 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO: 5 or SEQ ID NO:23, and a Type I-B CRISPR associated complex for antiviral defense complex (Cascade complex) comprising (i) a Cas6 polypeptide having at least 80% sequence identity to the amino acid sequence of SEQ ID NO:1 or SEQ ID NO:19, a Cas8 polypeptide having at least 80% sequence identity to the amino acid sequence of SEQ ID NO:2 or SEQ ID NO: 20, a Cas7 polypeptide having at least 80% sequence identity to the amino acid sequence of SEQ ID NO:3 or SEQ ID NO:21, and a Cas5 polypeptide having at least 80% sequence identity to the amino acid sequence of SEQ ID NO:4 or SEQ ID NO:22,
wherein the recombinant nucleic acid construct comprising a CRISPR and the recombinant nucleic acid construct encoding a Cascade complex each comprise a polynucleotide encoding a polypeptide conferring resistance to a selection agent, thereby producing a subpopulation of cells of the population of cells; and
(b) selecting from the subpopulation of cells produced in (a) one or more cells that are resistant to the selection agent, thereby selecting one or more variant cells that do not comprise the target sequence and are not killed.

63. The method of claim 61 or claim 62, wherein the organism is a bacterium, optionally a commensal Clostridium spp.

64. A method of killing one or more cells in a population of bacterial and/or archaeal cells, the method comprising introducing into the one or more cells of the population of bacterial and/or archaeal cells a protein-RNA complex, the protein-RNA complex comprising:
(a) a Clustered Regularly Interspaced Short Palindromic Repeats (CRISPR) comprising one or more repeat sequences and one or more spacer nucleotide sequence(s),
wherein each of the one or more spacer sequences comprises a 3′ end and a 5′ end and is linked at least at its 5′ end to a repeat sequence or portion thereof, and each of the one or more spacer sequences is complementary to a target sequence (protospacer) in the genome of the bacterial and/or archaeal cells of the population,
wherein the target sequence is a genomic sequence that is conserved among the one or more cells within the population of bacterial and/or archaeal cells and the target sequence is located immediately adjacent (3′) to a protospacer adjacent motif (PAM); and
(b) a Cas3 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO: 5 or SEQ ID NO:23, and a Type I-B CRISPR associated complex for antiviral defense complex (Cascade complex) comprising (i) a Cas6 polypeptide having at least 80% sequence identity to the amino acid sequence of SEQ ID NO:1 or SEQ ID NO:19, a Cas8 polypeptide having at least 80% sequence identity to the amino acid sequence of SEQ ID NO:2 or SEQ ID NO: 20, a Cas7 polypeptide having at least 80% sequence identity to the amino acid sequence of SEQ ID NO:3 or SEQ ID NO:21, and a Cas5 polypeptide having at least 80% sequence identity to the amino acid sequence of SEQ ID NO:4 or SEQ ID NO:22, thereby killing one or more cells in the population of bacterial and/or archaeal cells that comprise the target sequence in their genome.

65. The method of any one of claims 59-64, wherein each of the one or more spacer sequences is linked at its 3′-end to a repeat sequence or portion thereof.

66. The method of any one of claims 36 to 65, wherein the one or more repeat sequences comprise at least 19 consecutive nucleotides (e.g., about 19, 20, 21, 22, 23, 24, 25, 26, 27, 28, 29, 30, 31, or 32 consecutive nucleotides) of any one of the nucleotide sequences of SEQ ID NO: 17 or SEQ ID NO:18, or any combination thereof, or any one of the nucleotide sequences of SEQ ID NOs: 35-43, or any combination thereof, optionally about 27 to 32 consecutive nucleotides or about 30 to 32 consecutive nucleotides.

67. The method of claim 66, wherein the one or more repeat sequences comprise the same sequence.

68. The method of any one of claims 36-67, wherein the PAM comprises a nucleotide sequence of 5′-TTTA-3′ 5′-ATC-3′, 5′-ATCG-3′ and/or 5′-ATCA-3′ that is immediately adjacent to and 5′ of the target sequence (protospacer).

69. The method of any one of claims 36-68, wherein the spacer sequence is at least 80% complementary to the target sequence (e.g., about 80, 81, 82, 83, 84, 85, 86, 87, 88, 89, 90, 91, 92, 93, 94, 95, 96, 97, 98, 99, or 100%).

70. The method of any one of claims 36-69, wherein the one or more spacer sequence(s) each have a length of about 25 nucleotides to about 60 nucleotides, optionally about 30 nucleotides to about 40 nucleotides, or about 34 to about 37 nucleotides.

71. The method of any one of claims 36-70, wherein at least two of the one or more spacer sequence(s) comprise nucleotide sequences that are complementary to different target sequences.

72. The recombinant nucleic acid construct of any one of claims 35-71, wherein the one or more spacer sequence(s) each comprise a 5′ region and a 3′ region, wherein the 5′ region comprises a seed sequence and the 3′ region comprises a remaining portion of the one or more spacer sequence(s).

73. The method of claim 72, wherein the seed sequence comprises the first 8 nucleotides of the 5′ end of each of the one or more spacer sequence(s), and is fully complementary (100%) to the target sequence, and the remaining portion of the one or more spacer sequence(s) is at least about 80% complementary (e.g., about 80, 81, 82, 83, 84, 85, 86, 87, 88, 89, 90, 91, 92, 93, 94, 95, 96, 97, 98, 99, or 100%; or at least about 85%, 90%, 95%, 96%, 97%, 98%, 99% sequence complementarity) to the target sequence.

74. The method of any one of claims 36-73, wherein the target sequence is located in a gene, optionally in the upper (sense, coding) strand or in the bottom (antisense, non-coding) strand.

75. The method of claim 74, wherein the target sequence is located in an intragenic region of the gene (e.g., an intron), optionally located in the upper (sense, coding) strand or in the bottom (antisense, non-coding) strand.

76. The method of any one of claims 36-75, wherein the target sequence is located in an intergenic region, optionally in the upper (plus) strand or in the bottom (minus) strand.

77. The method of any one of claims 36-76, wherein the target sequence is located on a chromosome.

78. The method of any one of claims 36-76, wherein the target sequence is located on extrachromosomal nucleic acid.

79. The method of any one of claim 36-76 or 78, wherein the target sequence is located on a plasmid.

80. The method of any one of claims 74-79, wherein the gene encodes a transcription factor or a promoter.

81. The method of any one of claims 74-79, wherein the gene encodes non-coding RNA (e.g., miRNA, siRNA, piRNA (piwi-interacting RNA) or lncRNA (long non-coding RNA)).

82. The method of any one of claim 36, 39, 40, 49-56, 59, 61, 62, or 65-81, wherein the target organism is a eukaryote, a prokaryote, or a virus.

83. The method of claim 36, 38-40, 45-60, 61-63, or 65-81, wherein the target organism is a bacterium, an archaeon, an insect, a fungus, a plant, or an animal.
